(19) United States
(12) Patent Application Publication
(10) Pub. No.: US 2026/0070954 A1
(43) Pub. Date: Mar. 12, 2026

(54) USE OF A POLYPEPTIDE FOR THE PREVENTION OR TREATMENT OF ALLERGIC CONJUNCTIVITIS

(71) Applicant: Shanghai Puyou Biomedical Co., Ltd., Shanghai (CN)

(72) Inventors: Yinghao ZHANG, Shanghai (CN); Guirui YAN, Shanghai (CN)

(21) Appl. No.: 19/327,914

(22) Filed: Sep. 12, 2025

Related U.S. Application Data
(63) Continuation of application No. PCT/CN2024/081353, filed on Mar. 13, 2024.

(30) Foreign Application Priority Data
Mar. 14, 2023 (WO) PCT/CN2023/081353

(51) Int. Cl. C07K 14/47; A61K 38/00; A61P 27/14

(52) U.S. Cl. CPC C07K 14/4702; A61P 27/14; A61K 38/00; C07K 2319/00

(57) ABSTRACT

The present disclosure provides use of a polypeptide for the prevention or treatment of allergic conjunctivitis. Also provided is a method for treating or preventing allergic conjunctivitis in a subject in need thereof, comprising administering to the subject an effective amount of any one of the polypeptides disclosed herein.

### CROSS-REFERENCE

This application is a continuation of International Application No. PCT/CN2024/081353 filed on Mar. 13, 2024, which claims priority to International Application No. PCT/CN2023/081353 filed on Mar. 14, 2023, each of which is incorporated herein by reference in its entirety.

### SEQUENCE LISTING

The instant application contains a Sequence Listing which has been submitted electronically in XML format and is hereby incorporated by reference in its entirety. Said XML copy, created on Sep. 12, 2025, is named 64982-712_602_SL.xml and is 102,596 bytes in size.

### TECHNICAL FIELD

The present disclosure provides use of a polypeptide for the prevention or treatment of allergic conjunctivitis. Also provided is a method for treating or preventing allergic conjunctivitis in a subject in need thereof, comprising administering to the subject an effective amount of any one of the polypeptides disclosed herein.

### BACKGROUND

Conjunctiva is a thin, clear and mucous membrane that covers the inside of eyelids as well as the white part of the eyeball (i.e., sclera). The conjunctiva lubricates the eye by producing a small volume of mucus and tears, and in addition, it helps to prevent the microorganisms' entrance into the eye and also contributes to immune surveillance. Disorders of the conjunctiva are common among different populations, particularly because the surface of the eye is exposed to external influences directly and is easily affected by them.

Conjunctivitis (alternatively called “pink eye”), a very common type of disorder of the conjunctiva, is inflammation of the eye at the inner surface of the eyelid and the outermost layer of the white part.

Conjunctivitis is often manifested by symptoms such as redness and swelling of the conjunctiva, and watering of the eyes. This disorder may be caused by viral infection (i.e., viral conjunctivitis), bacterial infection (i.e., bacterial conjunctivitis), chemical substances (i.e., chemical conjunctivitis), allergens (i.e. allergic conjunctivitis), autoimmune response (i.e., autoimmune conjunctivitis, such as Sjogren syndrome, Stevens-Johnson syndrome and cicatricial pemphigoid), and so on.

Allergic conjunctivitis, among others, is reported to be the most frequent cause of conjunctivitis, affecting about 15% to 40% of the population (see Mourad M S, Rihan R A (April 2018). “Prevalence of Different Eye Diseases excluding Refractive Errors Presented at the Outpatient Clinic in Beheira Eye Hospital”. The Egyptian Journal of Hospital Medicine. 71 (2): 2484-2489). Allergic conjunctivitis is a class of inflammatory diseases caused by hypersensitivity of conjunctiva to the stimulations of various allergens. This disorder is essentially an immune reaction of conjunctiva to various of indoor or outdoor allergens such as pollens, mold spores, dust mites and pet dander that get into the eyes. As the eyes are exposed to the external environment directly and are relatively sensitive to various irritants, they are easily affected by allergens. The body releases chemical substances, including histamines which produce the inflammation in response to these allergens, and then develop allergic conjunctivitis.

Although allergic conjunctivitis is not a contagious disorder and is not life-threatening at all, it brings about considerable inconvenience to the lives of patients who are affected by this disorder and is sometimes rather tough to handle. Some common signs and symptoms of allergic conjunctivitis are, among others, redness, edema or hyperemia of conjunctiva, redness, edema or hyperemia of eyelids, ocular itching, visible damage of cornea, mucous discharge, watery discharge, tearing, photophobia, foreign body sensation and difficulty in opening eyes. It is also reported that patients may experience some other conditions such as itchy nose, sneezing, coughing, briefly blurred vision, distraction in mind, decreased productivity and tiredness.

The current clinical measures for caring allergic conjunctivitis include administration of eye drops or other ophthalmic formulations comprising antihistamines, mast cell stabilizers, glucocorticoids and/or immunosuppressants, which help to reduce the signs and symptoms of allergic conjunctivitis. However, these measures suffer from the problems such as slow onset, limited efficacy and/or duration, induction of ocular complications, poor treatment compliance, inconsistent dosing and drug abuse. As such, there still remains an unmet need in the medical community for drugs that effectively alleviate or treat allergic conjunctivitis.

### SUMMARY

The present disclosure provides new measures for preventing or treating allergic conjunctivitis. Specifically, the present disclosure provides use of a polypeptide for the prevention or treatment of allergic conjunctivitis. Also provided is a method for treating or preventing allergic conjunctivitis in a subject in need thereof, comprising administering to the subject an effective amount of any one of the polypeptides disclosed herein.

In one aspect, provided is a method for treating or preventing allergic conjunctivitis in a subject in need thereof, comprising administering to the subject an effective amount of an artificial polypeptide of formula (I):

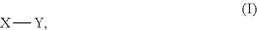

wherein X is a moiety comprising a sequence having at least 80% identity with SEQ ID NO: 1, and
Y is a moiety comprising 10 to 50 amino acids, at least 50% of which are selected from I, V, L, F, C, M, and A;
wherein Y comprises a total number of Cysteine (C) of less than 5.

In some embodiments, X comprises a sequence having at least 90% identity with SEQ ID NO: 1, X comprises a sequence having at least 95% identity with SEQ ID NO: 1, or X comprises a sequence of SEQ ID NO: 1. In some embodiments, X comprises a sequence having at least 90% identity with SEQ ID NO: 1. In some embodiments, X comprises a sequence having at least 95% identity with SEQ ID NO: 1. In some embodiments, X comprises a sequence of SEQ ID NO: 1. In some embodiments, at least 50% amino acids of X are selected from R, K, N, D, Q, E, and H. In some embodiments, X comprises a sequence having at least 90% identity with any one selected from SEQ ID NOs: 81-83. In some embodiments, Y comprises a total number of Cysteine (C) of less than 4, Y comprises a total number of Cysteine (C) of less than 3, or Y comprises a total number of Cysteine (C) of less than 2. In some embodiments, Y comprises a total number of Cysteine (C) of less than 4. In some embodiments, Y comprises a total number of Cysteine (C) of less than 3. In some embodiments, Y comprises a total number of Cysteine (C) of less than 2. In some embodiments, a total number of hydrophobic amino acids in Y is more than 8, the total number of hydrophobic amino acids in Y is more than 12, the total number of hydrophobic amino acids in Y is more than 15, and/or a total number of hydrophilic amino acids in Y is no more than 5. In some embodiments, a total number of hydrophobic amino acids in Y is more than 8. In some embodiments, the total number of hydrophobic amino acids in Y is more than 12. In some embodiments, the total number of hydrophobic amino acids in Y is more than 15. In some embodiments, a total number of hydrophilic amino acids in Y is no more than 5. In some embodiments, Y comprises a sequence having at least 90% identity with any one selected from SEQ ID NOs: 3-15, 58-61 and 84-85, or Y comprises a sequence having at least 90% identity with any one selected from SEQ ID NOs: 3-18, 58-61 and 84-85. In some embodiments, Y comprises a sequence having at least 90% identity with any one selected from SEQ ID NOs: 3-15, 58-61 and 84-85. In some embodiments, Y comprises a sequence having at least 90% identity with any one selected from SEQ ID NOs: 3-18, 58-61 and 84-85.

In some embodiments, the polypeptide comprises a sequence having at least 90% identity with any one selected from SEQ ID NOs: 29-41, 54-57, 75 and 91-95.

In some embodiments, the allergic conjunctivitis is at least one selected from the group consisting of seasonal allergic conjunctivitis, perennial allergic conjunctivitis, vernal keratoconjunctivitis, giant papillary conjunctivitis, atopic keratoconjunctivitis and phlyctenular keratoconjunctivitis. In some embodiments, the seasonal allergic conjunctivitis is caused or partially caused by a seasonal allergen. In some embodiments, the seasonal allergen is at least one selected from the group consisting of tree pollens, weed pollens, grass pollens and mold spores. In some embodiments, the perennial allergic conjunctivitis is caused or partially caused by a perennial allergen. In some embodiments, the perennial allergen is at least one selected from the group consisting of dust mites, cockroaches and animal danders. In some embodiments, the vernal keratoconjunctivitis is at least one selected from the group consisting of palpebral vernal keratoconjunctivitis, limbal vernal keratoconjunctivitis and mixed vernal keratoconjunctivitis. In some embodiments, the giant papillary conjunctivitis is caused or partially caused by repeated mechanical irritation of the conjunctiva. In some embodiments, the repeated mechanical irritation of the conjunctiva is associated with at least one selected from the group consisting of contact lenses wearing, use of ocular prostheses, use of conjunctival sutures, use of extruding scleral buckles and elevated corneal scars. In some embodiments, the atopic keratoconjunctivitis is companied with atopic dermatitis and/or asthma. In some embodiments, the phlyctenular keratoconjunctivitis is caused or partially caused by a delayed hypersensitivity or inflammatory reaction of the cornea and conjunctiva to antigens expressed by various pathogens. In some embodiments, the pathogens are at least one selected from the group consisting of bacteria (e.g., Staphylococcus aureus, Streptococcus viridians, Mycobacterium tuberculosis), viruses (e.g., Herpes simplex virus), chlamydiae, fungi (e.g., Candida such as Candida albicans) and parasites (e.g., tapeworms such as Hymenolepis nana; and nematodes). In some embodiments, the subject does not have any ocular disease selected from the group consisting of viral conjunctivitis, bacterial conjunctivitis, chemical conjunctivitis and dry eye or dry eye associated disorders. In some embodiments, the subject experiences at least one symptom selected from the group consisting of redness of conjunctiva, edema of conjunctiva, hyperemia of conjunctiva, redness of eyelids, edema of eyelids, hyperemia of eyelids, ocular itching, visible damage of cornea, mucous discharge, watery discharge, tearing, photophobia, foreign body sensation and difficulty in opening eyes before administrating of the artificial polypeptide of formula (I). In some embodiments, the subject displays reduction in at least one symptom selected from the group consisting of redness of conjunctiva, edema of conjunctiva, hyperemia of conjunctiva, redness of eyelids, edema of eyelids, hyperemia of eyelids, ocular itching, visible damage of cornea, mucous discharge, watery discharge, tearing, photophobia, foreign body sensation and difficulty in opening eyes after administrating of at least one dose of the artificial polypeptide of formula (I). In some embodiments, the subject displays improvements in at least one tissue selected from the group consisting of conjunctiva, cornea, anterior chamber, iris, pupil, vitreum vitreous and fundus after administrating of at least one dose of the artificial polypeptide of formula (I).

In another aspect, provided is a method for treating or preventing allergic conjunctivitis in a subject in need thereof, comprising administering to the subject an effective amount of an artificial polypeptide of formula (II):

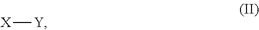

wherein X is a moiety comprising a sequence having at least 80% identity with SEQ ID NO: 1, and
Y is a moiety comprising 10 to 50 amino acids, at least 50% of which are selected from I, V, L, F, C, M, and A;
wherein Y comprises a sequence having at most 90% identity with SEQ ID NO: 2.

In some embodiments, X comprises a sequence having at least 90% identity with SEQ ID NO: 1, X comprises a sequence having at least 95% identity with SEQ ID NO: 1, or X comprises a sequence of SEQ ID NO: 1. In some embodiments, X comprises a sequence having at least 90% identity with SEQ ID NO: 1. In some embodiments, X comprises a sequence having at least 95% identity with SEQ ID NO: 1. In some embodiments, X comprises a sequence of SEQ ID NO: 1. In some embodiments, at least 50% amino acids of X are selected from R, K, N, D, Q, E, and H.

In some embodiments, Y comprises a sequence having at most 80% identity with SEQ ID NO: 2, Y comprises a sequence having at most 70% identity with SEQ ID NO: 2, or Y comprises a sequence having at most 50% identity with SEQ ID NO: 2. In some embodiments, Y comprises a sequence having at most 80% identity with SEQ ID NO: 2. In some embodiments, Y comprises a sequence having at most 70% identity with SEQ ID NO: 2. In some embodiments, Y comprises a sequence having at most 50% identity with SEQ ID NO: 2. In some embodiments, a total number of hydrophobic amino acids in Y is more than 8, the total number of hydrophobic amino acids in Y is more than 12, the total number of hydrophobic amino acids in Y is more than 15, and/or a total number of hydrophilic amino acids in Y is no more than 5. In some embodiments, a total number of hydrophobic amino acids in Y is more than 8. In some embodiments, the total number of hydrophobic amino acids in Y is more than 12. In some embodiments, the total number of hydrophobic amino acids in Y is more than 15. In some embodiments, a total number of hydrophilic amino acids in Y is no more than 5. In some embodiments, Y comprises a sequence having at least 90% identity with any one selected from SEQ ID NOs: 16-18, or Y comprises a sequence having at least 95% identity with any one selected from SEQ ID NOs: 16-18. In some embodiments, Y comprises a sequence having at least 90% identity with any one selected from SEQ ID NOs: 16-18. In some embodiments, Y comprises a sequence having at least 95% identity with any one selected from SEQ ID NOs: 16-18.

In some embodiments, the polypeptide comprises a sequence having at least 90% identity with any one selected from SEQ ID NOs: 42-44.

In some embodiments, the allergic conjunctivitis is at least one selected from the group consisting of seasonal allergic conjunctivitis, perennial allergic conjunctivitis, vernal keratoconjunctivitis, giant papillary conjunctivitis, atopic keratoconjunctivitis and phlyctenular keratoconjunctivitis. In some embodiments, the seasonal allergic conjunctivitis is caused or partially caused by a seasonal allergen. In some embodiments, the seasonal allergen is at least one selected from the group consisting of tree pollens, weed pollens, grass pollens and mold spores. In some embodiments, the perennial allergic conjunctivitis is caused or partially caused by a perennial allergen. In some embodiments, the perennial allergen is at least one selected from the group consisting of dust mites, cockroaches and animal danders. In some embodiments, the vernal keratoconjunctivitis is at least one selected from the group consisting of palpebral vernal keratoconjunctivitis, limbal vernal keratoconjunctivitis and mixed vernal keratoconjunctivitis. In some embodiments, the giant papillary conjunctivitis is caused or partially caused by repeated mechanical irritation of the conjunctiva. In some embodiments, the repeated mechanical irritation of the conjunctiva is associated with at least one selected from the group consisting of contact lenses wearing, use of ocular prostheses, use of conjunctival sutures, use of extruding scleral buckles and elevated corneal scars. In some embodiments, the atopic keratoconjunctivitis is companied with atopic dermatitis and/or asthma. In some embodiments, the phlyctenular keratoconjunctivitis is caused or partially caused by a delayed hypersensitivity or inflammatory reaction of the cornea and conjunctiva to antigens expressed by various pathogens. In some embodiments, the pathogens are at least one selected from the group consisting of bacteria (e.g., Staphylococcus aureus, Streptococcus viridians, Mycobacterium tuberculosis), viruses (e.g., Herpes simplex virus), chlamydiae, fungi (e.g., Candida such as Candida albicans) and parasites (e.g., tapeworms such as Hymenolepis nana; and nematodes). In some embodiments, the subject does not have any ocular disease selected from the group consisting of viral conjunctivitis, bacterial conjunctivitis, chemical conjunctivitis and dry eye or dry eye associated disorders. In some embodiments, the subject experiences at least one symptom selected from the group consisting of redness of conjunctiva, edema of conjunctiva, hyperemia of conjunctiva, redness of eyelids, edema of eyelids, hyperemia of eyelids, ocular itching, visible damage of cornea, mucous discharge, watery discharge, tearing, photophobia, foreign body sensation and difficulty in opening eyes before administrating of the artificial polypeptide of formula (II). In some embodiments, the subject displays reduction in at least one symptom selected from the group consisting of redness of conjunctiva, edema of conjunctiva, hyperemia of conjunctiva, redness of eyelids, edema of eyelids, hyperemia of eyelids, ocular itching, visible damage of cornea, mucous discharge, watery discharge, tearing, photophobia, foreign body sensation and difficulty in opening eyes after administrating of at least one dose of the artificial polypeptide of formula (II). In some embodiments, the subject displays improvements in at least one tissue selected from the group consisting of conjunctiva, cornea, anterior chamber, iris, pupil, vitreum vitreous and fundus after administrating of at least one dose of the artificial polypeptide of formula (II).

In another aspect, provided is a method for treating or preventing allergic conjunctivitis in a subject in need thereof, comprising administering to the subject an effective amount of an artificial polypeptide of formula (III):

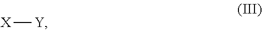

wherein X is a moiety comprising 40 to 65 amino acids, at least 50% of which are selected from H, R, K, D, Q, N and E; and
Y is a moiety comprising a sequence having at least 80% identity with SEQ ID NO: 2,
wherein a total number of H, R, K, D, Q, N and E in X is less than 33.

In some embodiments, Y comprises a sequence having at least 90% identity with SEQ ID NO: 2, Y comprises a sequence having at least 95% identity with SEQ ID NO: 2, or Y comprises a sequence of SEQ ID NO: 2. In some embodiments, Y comprises a sequence having at least 90% identity with SEQ ID NO: 2. In some embodiments, Y comprises a sequence having at least 95% identity with SEQ ID NO: 2. In some embodiments, Y comprises a sequence of SEQ ID NO: 2. In some embodiments, at least 50% amino acids of Y are selected from I, V, L, F, C, M, and A.

In some embodiments, wherein the total number of H, R, K, D, Q, N and E in X is less than 30, the total number of H, R, K, D, Q, N and E in X is less than 25, or the total number of H, R, K, D, Q, N and E in X is less than 20. In some embodiments, the total number of H, R, K, D, Q, N and E in X is less than 30.

In some embodiments, the total number of H, R, K, D, Q, N and E in X is less than 25. In some embodiments, the total number of H, R, K, D, Q, N and E in X is less than 20. In some embodiments, a total number of hydrophilic amino acids in X is more than 10, a total number of hydrophilic amino acids in X is more than 15, a total number of hydrophilic amino acids in X is more than 20, a total number of hydrophilic amino acids in X is more than 25, a total number of hydrophobic amino acids in X is no more than 15, and/or the total number of hydrophobic amino acids in X is no more than 10. In some embodiments, a total number of hydrophilic amino acids in X is more than 10. In some embodiments, a total number of hydrophilic amino acids in X is more than 15. In some embodiments, a total number of hydrophilic amino acids in X is more than 20. In some embodiments, a total number of hydrophilic amino acids in X is more than 25. In some embodiments, a total number of hydrophobic amino acids in X is no more than 15. In some embodiments, the total number of hydrophobic amino acids in X is no more than 10. In some embodiments, X comprises a sequence having at least 90% identity with any one selected from SEQ ID NOs: 23-27, or X comprises a sequence having at least 95% identity with any one selected from SEQ ID NOs: 23-27. In some embodiments, X comprises a sequence having at least 90% identity with any one selected from SEQ ID NOs: 23-27. In some embodiments, X comprises a sequence having at least 95% identity with any one selected from SEQ ID NOs: 23-27.

In some embodiments, the polypeptide comprises a sequence having at least 90% identity with SEQ ID NOs: 49-53.

In some embodiments, the allergic conjunctivitis is at least one selected from the group consisting of seasonal allergic conjunctivitis, perennial allergic conjunctivitis, vernal keratoconjunctivitis, giant papillary conjunctivitis, atopic keratoconjunctivitis and phlyctenular keratoconjunctivitis. In some embodiments, the seasonal allergic conjunctivitis is caused or partially caused by a seasonal allergen. In some embodiments, the seasonal allergen is at least one selected from the group consisting of tree pollens, weed pollens, grass pollens and mold spores. In some embodiments, the perennial allergic conjunctivitis is caused or partially caused by a perennial allergen. In some embodiments, the perennial allergen is at least one selected from the group consisting of dust mites, cockroaches and animal danders. In some embodiments, the vernal keratoconjunctivitis is at least one selected from the group consisting of palpebral vernal keratoconjunctivitis, limbal vernal keratoconjunctivitis and mixed vernal keratoconjunctivitis. In some embodiments, the giant papillary conjunctivitis is caused or partially caused by repeated mechanical irritation of the conjunctiva. In some embodiments, the repeated mechanical irritation of the conjunctiva is associated with at least one selected from the group consisting of contact lenses wearing, use of ocular prostheses, use of conjunctival sutures, use of extruding scleral buckles and elevated corneal scars. In some embodiments, the atopic keratoconjunctivitis is companied with atopic dermatitis and/or asthma. In some embodiments, the phlyctenular keratoconjunctivitis is caused or partially caused by a delayed hypersensitivity or inflammatory reaction of the cornea and conjunctiva to antigens expressed by various pathogens. In some embodiments, the pathogens are at least one selected from the group consisting of bacteria (e.g., Staphylococcus aureus, Streptococcus viridians, Mycobacterium tuberculosis), viruses (e.g., Herpes simplex virus), chlamydiae, fungi (e.g., Candida such as Candida albicans) and parasites (e.g., tapeworms such as Hymenolepis nana; and nematodes). In some embodiments, the subject does not have any ocular disease selected from the group consisting of viral conjunctivitis, bacterial conjunctivitis, chemical conjunctivitis and dry eye or dry eye associated disorders. In some embodiments, the subject experiences at least one symptom selected from the group consisting of redness of conjunctiva, edema of conjunctiva, hyperemia of conjunctiva, redness of eyelids, edema of eyelids, hyperemia of eyelids, ocular itching, visible damage of cornea, mucous discharge, watery discharge, tearing, photophobia, foreign body sensation and difficulty in opening eyes before administrating of the artificial polypeptide of formula (III). In some embodiments, the subject displays reduction in at least one symptom selected from the group consisting of redness of conjunctiva, edema of conjunctiva, hyperemia of conjunctiva, redness of eyelids, edema of eyelids, hyperemia of eyelids, ocular itching, visible damage of cornea, mucous discharge, watery discharge, tearing, photophobia, foreign body sensation and difficulty in opening eyes after administrating of at least one dose of the artificial polypeptide of formula (III). In some embodiments, the subject displays improvements in at least one tissue selected from the group consisting of conjunctiva, cornea, anterior chamber, iris, pupil, vitreum vitreous and fundus after administrating of at least one dose of the artificial polypeptide of formula (III).

In another aspect, provided is a method for treating or preventing allergic conjunctivitis in a subject in need thereof, comprising administering to the subject an effective amount of an artificial polypeptide of formula (IV):

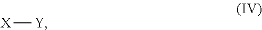

wherein X is a moiety comprising 40 to 65 amino acids, at least 50% of which are selected from H, R, K, D, Q, N and E; and
Y is a moiety comprising a sequence having at least 80% identity with SEQ ID NO: 2,
wherein X comprises a sequence having at most 90% identity with SEQ ID NO: 1.

In some embodiments, X comprises a sequence having at most 80% identity with SEQ ID NO: 1, X comprises a sequence having at most 70% identity with SEQ ID NO: 1, or X comprises a sequence having at most 50% identity with SEQ ID NO: 1. In some embodiments, X comprises a sequence having at most 80% identity with SEQ ID NO: 1. In some embodiments, X comprises a sequence having at most 70% identity with SEQ ID NO: 1. In some embodiments, X comprises a sequence having at most 50% identity with SEQ ID NO: 1. In some embodiments, a total number of hydrophilic amino acids in X is more than 10, a total number of hydrophilic amino acids in X is more than 15, a total number of hydrophilic amino acids in X is more than 20, a total number of hydrophilic amino acids in X is more than 25, a total number of hydrophobic amino acids in X is no more than 15, and/or the total number of hydrophobic amino acids in X is no more than 10. In some embodiments, a total number of hydrophilic amino acids in X is more than 10. In some embodiments, a total number of hydrophilic amino acids in X is more than 15. In some embodiments, a total number of hydrophilic amino acids in X is more than 20. In some embodiments, a total number of hydrophilic amino acids in X is more than 25. In some embodiments, a total number of hydrophobic amino acids in X is no more than 15. In some embodiments, the total number of hydrophobic amino acids in X is no more than 10. In some embodiments, X comprises a sequence having at least 90% identity with any one selected from SEQ ID NOs:19-22 and 76-79, or X comprises a sequence having at least 95% identity with any one selected from SEQ ID NOs: 19-22 and 76-79. In some embodiments, X comprises a sequence having at least 90% identity with any one selected from SEQ ID NOs:19-22 and 76-79. In some embodiments, X comprises a sequence having at least 95% identity with any one selected from SEQ ID NOs: 19-22 and 76-79.

In some embodiments, Y comprises a sequence having at least 90% identity with SEQ ID NO: 2, Y comprises a sequence having at least 95% identity with SEQ ID NO: 2, or Y comprises a sequence of SEQ ID NO: 2. In some embodiments, Y comprises a sequence having at least 90% identity with SEQ ID NO: 2. In some embodiments, Y comprises a sequence having at least 95% identity with SEQ ID NO: 2. In some embodiments, Y comprises a sequence of SEQ ID NO: 2. In some embodiments, at least 50% amino acids of Y are selected from I, V, L, F, C, M, and A.

In some embodiments, the polypeptide comprises a sequence having at least 90% identity with any one selected from SEQ ID NOs:45-48 and 86-89.

In some embodiments, the allergic conjunctivitis is at least one selected from the group consisting of seasonal allergic conjunctivitis, perennial allergic conjunctivitis, vernal keratoconjunctivitis, giant papillary conjunctivitis, atopic keratoconjunctivitis and phlyctenular keratoconjunctivitis. In some embodiments, the seasonal allergic conjunctivitis is caused or partially caused by a seasonal allergen. In some embodiments, the seasonal allergen is at least one selected from the group consisting of tree pollens, weed pollens, grass pollens and mold spores. In some embodiments, the perennial allergic conjunctivitis is caused or partially caused by a perennial allergen. In some embodiments, the perennial allergen is at least one selected from the group consisting of dust mites, cockroaches and animal danders. In some embodiments, the vernal keratoconjunctivitis is at least one selected from the group consisting of palpebral vernal keratoconjunctivitis, limbal vernal keratoconjunctivitis and mixed vernal keratoconjunctivitis. In some embodiments, the giant papillary conjunctivitis is caused or partially caused by repeated mechanical irritation of the conjunctiva. In some embodiments, the repeated mechanical irritation of the conjunctiva is associated with at least one selected from the group consisting of contact lenses wearing, use of ocular prostheses, use of conjunctival sutures, use of extruding scleral buckles and elevated corneal scars. In some embodiments, the atopic keratoconjunctivitis is companied with atopic dermatitis and/or asthma. In some embodiments, the phlyctenular keratoconjunctivitis is caused or partially caused by a delayed hypersensitivity or inflammatory reaction of the cornea and conjunctiva to antigens expressed by various pathogens. In some embodiments, the pathogens are at least one selected from the group consisting of bacteria (e.g., Staphylococcus aureus, Streptococcus viridians, Mycobacterium tuberculosis), viruses (e.g., Herpes simplex virus), chlamydiae, fungi (e.g., Candida such as Candida albicans) and parasites (e.g., tapeworms such as Hymenolepis nana; and nematodes). In some embodiments, the subject does not have any ocular disease selected from the group consisting of viral conjunctivitis, bacterial conjunctivitis, chemical conjunctivitis and dry eye or dry eye associated disorders. In some embodiments, the subject experiences at least one symptom selected from the group consisting of redness of conjunctiva, edema of conjunctiva, hyperemia of conjunctiva, redness of eyelids, edema of eyelids, hyperemia of eyelids, ocular itching, visible damage of cornea, mucous discharge, watery discharge, tearing, photophobia, foreign body sensation and difficulty in opening eyes before administrating of the artificial polypeptide of formula (IV). In some embodiments, the subject displays reduction in at least one symptom selected from the group consisting of redness of conjunctiva, edema of conjunctiva, hyperemia of conjunctiva, redness of eyelids, edema of eyelids, hyperemia of eyelids, ocular itching, visible damage of cornea, mucous discharge, watery discharge, tearing, photophobia, foreign body sensation and difficulty in opening eyes after administrating of at least one dose of the artificial polypeptide of formula (IV). In some embodiments, the subject displays improvements in at least one tissue selected from the group consisting of conjunctiva, cornea, anterior chamber, iris, pupil, vitreum vitreous and fundus after administrating of at least one dose of the artificial polypeptide of formula (IV).

In another aspect, provided is a method for treating or preventing allergic conjunctivitis in a subject in need thereof, comprising administering to the subject an effective amount of an artificial polypeptide of formula (V):

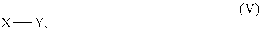

wherein X is a moiety comprising a sequence having at least 80% identity with SEQ ID NO: 1, and
Y is a moiety comprising 10 to 30 amino acids, wherein Y comprises a sequence having at least 10 continuous AAs of SEQ ID NO: 2.

In some embodiments, X comprises a sequence having at least 90% identity with SEQ ID NO: 1, X comprises a sequence having at least 95% identity with SEQ ID NO: 1, or X comprises a sequence of SEQ ID NO: 1. In some embodiments, X comprises a sequence having at least 90% identity with SEQ ID NO: 1. In some embodiments, X comprises a sequence having at least 95% identity with SEQ ID NO: 1.

In some embodiments, X comprises a sequence of SEQ ID NO: 1. In some embodiments, at least 50% amino acids of X are selected from R, K, N, D, Q, E, and H. In some embodiments, X comprises a sequence having at least 80% identity with SEQ ID NO: 96, X comprises a sequence having at least 90% identity with SEQ ID NO: 96, X comprises a sequence having at least 95% identity with SEQ ID NO: 96, or X comprises a sequence of SEQ ID NO: 96. In some embodiments, X comprises a sequence having at least 80% identity with SEQ ID NO: 96. In some embodiments, X comprises a sequence having at least 90% identity with SEQ ID NO: 96. In some embodiments, X comprises a sequence having at least 95% identity with SEQ ID NO: 96. In some embodiments, X comprises a sequence of SEQ ID NO: 96.

In some embodiments, Y comprises 10 to 25 amino acids, Y comprises 10 to 20 amino acids, or Y comprises 10 to 15 amino acids. In some embodiments, Y comprises 10 to 25 amino acids. In some embodiments, Y comprises 10 to 20 amino acids. In some embodiments, Y comprises 10 to 15 amino acids.

In some embodiments, Y comprises a sequence having at most 80% identity with SEQ ID NO: 2, Y comprises a sequence having at most 70% identity with SEQ ID NO: 2, or Y comprises a sequence having at most 50% identity with SEQ ID NO: 2. In some embodiments, Y comprises a sequence having at most 80% identity with SEQ ID NO: 2. In some embodiments, Y comprises a sequence having at most 70% identity with SEQ ID NO: 2. In some embodiments, Y comprises a sequence having at most 50% identity with SEQ ID NO: 2. In some embodiments, Y comprises a sequence having at least 80% identity with any one selected from SEQ ID NOs: 97-103, Y comprises a sequence having at least 90% identity with any one selected from SEQ ID NOs: 97-103, Y comprises a sequence having at least 95% identity with any one selected from SEQ ID NOs: 97-103, or Y comprises a sequence that is any one selected from SEQ ID NOs: 97-103. In some embodiments, Y comprises a sequence having at least 80% identity with any one selected from SEQ ID NOs: 97-103. In some embodiments, Y comprises a sequence having at least 90% identity with any one selected from SEQ ID NOs: 97-103. In some embodiments, Y comprises a sequence having at least 95% identity with any one selected from SEQ ID NOs: 97-103. In some embodiments, Y comprises a sequence that is any one selected from SEQ ID NOs: 97-103.

In some embodiments, the polypeptide comprises a sequence having at least 90% identity with any of SEQ ID NOs: 53-56 and 104-110.

In some embodiments, the allergic conjunctivitis is at least one selected from the group consisting of seasonal allergic conjunctivitis, perennial allergic conjunctivitis, vernal keratoconjunctivitis, giant papillary conjunctivitis, atopic keratoconjunctivitis and phlyctenular keratoconjunctivitis. In some embodiments, the seasonal allergic conjunctivitis is caused or partially caused by a seasonal allergen. In some embodiments, the seasonal allergen is at least one selected from the group consisting of tree pollens, weed pollens, grass pollens and mold spores. In some embodiments, the perennial allergic conjunctivitis is caused or partially caused by a perennial allergen. In some embodiments, the perennial allergen is at least one selected from the group consisting of dust mites, cockroaches and animal danders. In some embodiments, the vernal keratoconjunctivitis is at least one selected from the group consisting of palpebral vernal keratoconjunctivitis, limbal vernal keratoconjunctivitis and mixed vernal keratoconjunctivitis. In some embodiments, the giant papillary conjunctivitis is caused or partially caused by repeated mechanical irritation of the conjunctiva. In some embodiments, the repeated mechanical irritation of the conjunctiva is associated with at least one selected from the group consisting of contact lenses wearing, use of ocular prostheses, use of conjunctival sutures, use of extruding scleral buckles and elevated corneal scars. In some embodiments, the atopic keratoconjunctivitis is companied with atopic dermatitis and/or asthma. In some embodiments, the phlyctenular keratoconjunctivitis is caused or partially caused by a delayed hypersensitivity or inflammatory reaction of the cornea and conjunctiva to antigens expressed by various pathogens. In some embodiments, the pathogens are at least one selected from the group consisting of bacteria (e.g., Staphylococcus aureus, Streptococcus viridians, Mycobacterium tuberculosis), viruses (e.g., Herpes simplex virus), chlamydiae, fungi (e.g., Candida such as Candida albicans) and parasites (e.g., tapeworms such as Hymenolepis nana; and nematodes). In some embodiments, the subject does not have any ocular disease selected from the group consisting of viral conjunctivitis, bacterial conjunctivitis, chemical conjunctivitis and dry eye or dry eye associated disorders. In some embodiments, the subject experiences at least one symptom selected from the group consisting of redness of conjunctiva, edema of conjunctiva, hyperemia of conjunctiva, redness of eyelids, edema of eyelids, hyperemia of eyelids, ocular itching, visible damage of cornea, mucous discharge, watery discharge, tearing, photophobia, foreign body sensation and difficulty in opening eyes before administrating of the artificial polypeptide of formula (V). In some embodiments, the subject displays reduction in at least one symptom selected from the group consisting of redness of conjunctiva, edema of conjunctiva, hyperemia of conjunctiva, redness of eyelids, edema of eyelids, hyperemia of eyelids, ocular itching, visible damage of cornea, mucous discharge, watery discharge, tearing, photophobia, foreign body sensation and difficulty in opening eyes after administrating of at least one dose of the artificial polypeptide of formula (V). In some embodiments, the subject displays improvements in at least one tissue selected from the group consisting of conjunctiva, cornea, anterior chamber, iris, pupil, vitreum vitreous and fundus after administrating of at least one dose of the artificial polypeptide of formula (V).

In another aspect, provided is a method for treating or preventing allergic conjunctivitis in a subject in need thereof, comprising administering to the subject an effective amount of an artificial polypeptide of formula (VI):

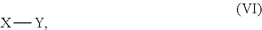

wherein X is a moiety comprising a mutant of the sequence SEQ ID NO: 1, characterized in that the mutant has at least 1, 2, 3, 4, 5, 6, 7, 8, or 9 D in SEQ ID NO: 1 mutated to S, and/or at least 1, 2, 3, 4, 5, or 6 E in SEQ ID NO: 1 mutated to T; and
Y is a moiety comprising a mutant of the sequence SEQ ID NO: 2, characterized in that the mutant has at least 1, 2, 3, 4, or 5 C in SEQ ID NO: 2 mutated to A.

In some embodiments, X comprises a sequence having at least 70% identity with SEQ ID NO: 1 and/or X comprises a sequence having at most 95% identity with SEQ ID NO: 1. In some embodiments, X comprises a sequence having at least 70% identity with SEQ ID NO: 1. In some embodiments, X comprises a sequence having at most 95% identity with SEQ ID NO: 1. In some embodiments, a total number of R, K, T, A, N, Q, D, E, S, and G in X is more than 30 and/or a total number of W, Y, F, M, L, I, and V in X is no more than 20. In some embodiments, a total number of R, K, T, A, N, Q, D, E, S, and G in X is more than 30. In some embodiments, a total number of W, Y, F, M, L, I, and V in X is no more than 20. In some embodiments, X comprises a sequence having at least 80% identity with any one selected from SEQ ID NOs: 80-83, X comprises a sequence that is any one selected from SEQ ID NOs: 80-83, or X is a sequence that is any one selected from SEQ ID NOs: 80-83. In some embodiments, X comprises a sequence having at least 80% identity with any one selected from SEQ ID NOs: 80-83. In some embodiments, X comprises a sequence that is any one selected from SEQ ID NOs: 80-83. In some embodiments, X is a sequence that is any one selected from SEQ ID NOs: 80-83.

In some embodiments, Y comprises a sequence having at least 80% identity with SEQ ID NO: 2 and/or Y comprises a sequence having at most 95% identity with SEQ ID NO: 2. In some embodiments, Y comprises a sequence having at least 80% identity with SEQ ID NO: 2. In some embodiments, Y comprises a sequence having at most 95% identity with SEQ ID NO: 2. In some embodiments, at least 50% amino acids of Y are selected from I, V, L, F, C, M, and A. In some embodiments, a total number of R, K, T, A, N, Q, D, E, S, and G in Y is more than 10 and/or a total number of W, Y, F, M, L, I, and V in Y is no more than 20. In some embodiments, a total number of R, K, T, A, N, Q, D, E, S, and G in Y is more than 10. In some embodiments, a total number of W, Y, F, M, L, I, and V in Y is no more than 20. In some embodiments, Y comprises a sequence having at least 80% identity with SEQ ID NO: 3, Y comprises a sequence of SEQ ID NO: 3, or Y is a sequence of SEQ ID NO: 3. In some embodiments, Y comprises a sequence having at least 80% identity with SEQ ID NO: 3. In some embodiments, Y comprises a sequence of SEQ ID NO: 3.

In some embodiments, Y is a sequence of SEQ ID NO: 3.

In some embodiments, the artificial polypeptide comprises a sequence having at least 80% identity with any one selected from SEQ ID NOs: 90-93, the artificial polypeptide comprises a sequence that is any one selected from SEQ ID NOs: 90-93, or the artificial polypeptide is a sequence that is any one selected from SEQ ID NOs: 90-93. In some embodiments, the artificial polypeptide comprises a sequence having at least 80% identity with any one selected from SEQ ID NOs: 90-93. In some embodiments, the artificial polypeptide comprises a sequence that is any one selected from SEQ ID NOs: 90-93. In some embodiments, the artificial polypeptide is a sequence that is any one selected from SEQ ID NOs: 90-93.

In some embodiments, the allergic conjunctivitis is at least one selected from the group consisting of seasonal allergic conjunctivitis, perennial allergic conjunctivitis, vernal keratoconjunctivitis, giant papillary conjunctivitis, atopic keratoconjunctivitis and phlyctenular keratoconjunctivitis. In some embodiments, the seasonal allergic conjunctivitis is caused or partially caused by a seasonal allergen. In some embodiments, the seasonal allergen is at least one selected from the group consisting of tree pollens, weed pollens, grass pollens and mold spores. In some embodiments, the perennial allergic conjunctivitis is caused or partially caused by a perennial allergen. In some embodiments, the perennial allergen is at least one selected from the group consisting of dust mites, cockroaches and animal danders. In some embodiments, the vernal keratoconjunctivitis is at least one selected from the group consisting of palpebral vernal keratoconjunctivitis, limbal vernal keratoconjunctivitis and mixed vernal keratoconjunctivitis. In some embodiments, the giant papillary conjunctivitis is caused or partially caused by repeated mechanical irritation of the conjunctiva. In some embodiments, the repeated mechanical irritation of the conjunctiva is associated with at least one selected from the group consisting of contact lenses wearing, use of ocular prostheses, use of conjunctival sutures, use of extruding scleral buckles and elevated corneal scars. In some embodiments, the atopic keratoconjunctivitis is companied with atopic dermatitis and/or asthma. In some embodiments, the phlyctenular keratoconjunctivitis is caused or partially caused by a delayed hypersensitivity or inflammatory reaction of the cornea and conjunctiva to antigens expressed by various pathogens. In some embodiments, the pathogens are at least one selected from the group consisting of bacteria (e.g., Staphylococcus aureus, Streptococcus viridians, Mycobacterium tuberculosis), viruses (e.g., Herpes simplex virus), chlamydiae, fungi (e.g., Candida such as Candida albicans) and parasites (e.g., tapeworms such as Hymenolepis nana; and nematodes). In some embodiments, the subject does not have any ocular disease selected from the group consisting of viral conjunctivitis, bacterial conjunctivitis, chemical conjunctivitis and dry eye or dry eye associated disorders. In some embodiments, the subject experiences at least one symptom selected from the group consisting of redness of conjunctiva, edema of conjunctiva, hyperemia of conjunctiva, redness of eyelids, edema of eyelids, hyperemia of eyelids, ocular itching, visible damage of cornea, mucous discharge, watery discharge, tearing, photophobia, foreign body sensation and difficulty in opening eyes before administrating of the artificial polypeptide of formula (VI). In some embodiments, the subject displays reduction in at least one symptom selected from the group consisting of redness of conjunctiva, edema of conjunctiva, hyperemia of conjunctiva, redness of eyelids, edema of eyelids, hyperemia of eyelids, ocular itching, visible damage of cornea, mucous discharge, watery discharge, tearing, photophobia, foreign body sensation and difficulty in opening eyes after administrating of at least one dose of the artificial polypeptide of formula (VI). In some embodiments, the subject displays improvements in at least one tissue selected from the group consisting of conjunctiva, cornea, anterior chamber, iris, pupil, vitreum vitreous and fundus after administrating of at least one dose of the artificial polypeptide of formula (VI).

In another aspect, provided is a method for treating or preventing allergic conjunctivitis in a subject in need thereof, comprising administering to the subject an effective amount of an artificial polypeptide of formula (VII):

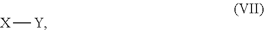

wherein X is a moiety comprising a mutant of the sequence SEQ ID NO: 1, characterized in that the mutant has at least 1, 2, 3, 4, or 5 amino acid insertions relative to SEQ ID NO: 1, and
Y is a moiety comprising 10 to 30 amino acids, wherein Y comprises a sequence having at least 15 continuous AAs of SEQ ID NO: 2.

In some embodiments, the amino acid insertions are located at the N-terminal of X and/or the inserted amino acid is M. In some embodiments, the amino acid insertions are located at the N-terminal of X. In some embodiments, the inserted amino acid is M.

In some embodiments, at least 50% amino acids of X are selected from R, K, N, D, Q, E, and H and/or at most 60% amino acids of X are selected from R, K, N, D, Q, E, and H. In some embodiments, at least 50% amino acids of X are selected from R, K, N, D, Q, E, and H. In some embodiments, at most 60% amino acids of X are selected from R, K, N, D, Q, E, and H. In some embodiments, a total number of E or D in X is at least 8 and/or a total number of E or D in X is at most 15. In some embodiments, a total number of E or D in X is at least 8. In some embodiments, a total number of E or D in X is at most 15. In some embodiments, a total number of R, K, T, A, N, Q, D, E, S, and G in X is more than 30 and/or a total number of W, Y, F, M, L, I, and V in X is no more than 20. In some embodiments, a total number of R, K, T, A, N, Q, D, E, S, and G in X is more than 30. In some embodiments, a total number of W, Y, F, M, L, I, and V in X is no more than 20. In some embodiments, X comprises a sequence having at least 80% identity with SEQ ID NO: 96, X comprises a sequence of SEQ ID NO: 96, or X is a sequence of SEQ ID NO: 96. In some embodiments, X comprises a sequence having at least 80% identity with SEQ ID NO: 96. In some embodiments, X comprises a sequence of SEQ ID NO: 96. In some embodiments, X is a sequence of SEQ ID NO: 96.

In some embodiments, Y is a moiety comprising a mutant of the sequence SEQ ID NO: 2, characterized in that the mutant has at least 1, 2, 3, 4, 5, 6, 7, 8, 9, 10, 11, 12, 13, 14, 15, 16, 17, 18, 19, or 20 amino acid truncations relative to SEQ ID NO: 2 and optionally the at least 1, 2, 3, 4, 5, 6, 7, 8, 9, 10, 11, 12, 13, 14, 15, 16, 17, 18, 19, or 20 amino acid truncations are located at the C-terminal of Y. In some embodiments, Y is a moiety comprising a mutant of the sequence SEQ ID NO: 2, characterized in that the mutant has at least 1, 2, 3, 4, 5, 6, 7, 8, 9, 10, 11, 12, 13, 14, 15, 16, 17, 18, 19, or 20 amino acid truncations relative to SEQ ID NO: 2. In some embodiments, the at least 1, 2, 3, 4, 5, 6, 7, 8, 9, 10, 11, 12, 13, 14, 15, 16, 17, 18, 19, or 20 amino acid truncations are located at the C-terminal of Y. In some embodiments, Y comprises 15 to 25 amino acids, Y comprises 18 to 25 amino acids, or Y comprises 20 to 25 amino acids.

In some embodiments, Y comprises 15 to 25 amino acids. In some embodiments, Y comprises 18 to 25 amino acids. In some embodiments, Y comprises 20 to 25 amino acids. In some embodiments, 40-65% amino acids of Y are selected from I, V, L, F, C, M, and A. In some embodiments, a total number of R, K, T, A, N, Q, D, E, S, and G in Y is no more than 10 and/or a total number of W, Y, F, M, L, I, and V in Y is no more than 15. In some embodiments, a total number of R, K, T, A, N, Q, D, E, S, and G in Y is no more than 10. In some embodiments, a total number of W, Y, F, M, L, I, and V in Y is no more than 15. In some embodiments, Y comprises a sequence having at least 80% identity with any one selected from SEQ ID NOs: 97-103, Y comprises a sequence that is any one selected from SEQ ID NOs: 97-103, or Y is a sequence that is any one selected from SEQ ID NOs: 97-103. In some embodiments, Y comprises a sequence having at least 80% identity with any one selected from SEQ ID NOs: 97-103. In some embodiments, Y comprises a sequence that is any one selected from SEQ ID NOs: 97-103. In some embodiments, Y is a sequence that is any one selected from SEQ ID NOs: 97-103.

In some embodiments, the artificial polypeptide comprises a sequence having at least 80% identity with any one selected from SEQ ID NOs: 104-110, the artificial polypeptide comprises a sequence that is any one selected from SEQ ID NOs: 104-110, or the artificial polypeptide is a sequence that is any one selected from SEQ ID NOs: 104-110. In some embodiments, the artificial polypeptide comprises a sequence having at least 80% identity with any one selected from SEQ ID NOs: 104-110. In some embodiments, the artificial polypeptide comprises a sequence that is any one selected from SEQ ID NOs: 104-110. In some embodiments, the artificial polypeptide is a sequence that is any one selected from SEQ ID NOs: 104-110.

In some embodiments, the allergic conjunctivitis is at least one selected from the group consisting of seasonal allergic conjunctivitis, perennial allergic conjunctivitis, vernal keratoconjunctivitis, giant papillary conjunctivitis, atopic keratoconjunctivitis and phlyctenular keratoconjunctivitis. In some embodiments, the seasonal allergic conjunctivitis is caused or partially caused by a seasonal allergen. In some embodiments, the seasonal allergen is at least one selected from the group consisting of tree pollens, weed pollens, grass pollens and mold spores. In some embodiments, the perennial allergic conjunctivitis is caused or partially caused by a perennial allergen. In some embodiments, the perennial allergen is at least one selected from the group consisting of dust mites, cockroaches and animal danders. In some embodiments, the vernal keratoconjunctivitis is at least one selected from the group consisting of palpebral vernal keratoconjunctivitis, limbal vernal keratoconjunctivitis and mixed vernal keratoconjunctivitis. In some embodiments, the giant papillary conjunctivitis is caused or partially caused by repeated mechanical irritation of the conjunctiva. In some embodiments, the repeated mechanical irritation of the conjunctiva is associated with at least one selected from the group consisting of contact lenses wearing, use of ocular prostheses, use of conjunctival sutures, use of extruding scleral buckles and elevated corneal scars. In some embodiments, the atopic keratoconjunctivitis is companied with atopic dermatitis and/or asthma. In some embodiments, the phlyctenular keratoconjunctivitis is caused or partially caused by a delayed hypersensitivity or inflammatory reaction of the cornea and conjunctiva to antigens expressed by various pathogens. In some embodiments, the pathogens are at least one selected from the group consisting of bacteria (e.g., Staphylococcus aureus, Streptococcus viridians, Mycobacterium tuberculosis), viruses (e.g., Herpes simplex virus), chlamydiae, fungi (e.g., Candida such as Candida albicans) and parasites (e.g., tapeworms such as Hymenolepis nana; and nematodes). In some embodiments, the subject does not have any ocular disease selected from the group consisting of viral conjunctivitis, bacterial conjunctivitis, chemical conjunctivitis and dry eye or dry eye associated disorders. In some embodiments, the subject experiences at least one symptom selected from the group consisting of redness of conjunctiva, edema of conjunctiva, hyperemia of conjunctiva, redness of eyelids, edema of eyelids, hyperemia of eyelids, ocular itching, visible damage of cornea, mucous discharge, watery discharge, tearing, photophobia, foreign body sensation and difficulty in opening eyes before administrating of the artificial polypeptide of formula (VII). In some embodiments, the subject displays reduction in at least one symptom selected from the group consisting of redness of conjunctiva, edema of conjunctiva, hyperemia of conjunctiva, redness of eyelids, edema of eyelids, hyperemia of eyelids, ocular itching, visible damage of cornea, mucous discharge, watery discharge, tearing, photophobia, foreign body sensation and difficulty in opening eyes after administrating of at least one dose of the artificial polypeptide of formula (VII). In some embodiments, the subject displays improvements in at least one tissue selected from the group consisting of conjunctiva, cornea, anterior chamber, iris, pupil, vitreum vitreous and fundus after administrating of at least one dose of the artificial polypeptide of formula (VII).

In another aspect, provided is a method for treating or preventing allergic conjunctivitis in a subject in need thereof, comprising administering to the subject an effective amount of a mutant of artificial polypeptide of formula (I),

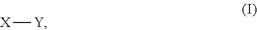

wherein X is a moiety comprising a sequence having at least 80% identity with SEQ ID NO: 1,
Y is a moiety comprising 10 to 50 amino acids, at least 50% of which are selected from I, V, L, F, C, M, and A,
Y comprises a total number of Cysteine (C) of less than 5,
characterized in that the mutant has at least 1, 2, 3, 4, 5, 6, 7, 8, or 9 D in X mutated to S, and/or at least 1, 2, 3, 4, 5, or 6 E in X mutated to T.

In some embodiments, at least 20% amino acids of X are selected from R, K, N, D, Q, E, and H, at least 30% amino acids of X are selected from R, K, N, D, Q, E, and H, at most 90% amino acids of X are selected from R, K, N, D, Q, E, and H, and/or at most 80% amino acids of X are selected from R, K, N, D, Q, E, and H. In some embodiments, at least 20% amino acids of X are selected from R, K, N, D, Q, E, and H. In some embodiments, at least 30% amino acids of X are selected from R, K, N, D, Q, E, and H. In some embodiments, at most 90% amino acids of X are selected from R, K, N, D, Q, E, and H. In some embodiments, at most 80% amino acids of X are selected from R, K, N, D, Q, E, and H. In some embodiments, X comprises a sequence having at least 70% identity with SEQ ID NO: 1.

In some embodiments, Y comprises a total number of Cysteine (C) of less than 4, or Y comprises a total number of Cysteine (C) of less than 3. In some embodiments, Y comprises a total number of Cysteine (C) of less than 4. In some embodiments, Y comprises a total number of Cysteine (C) of less than 3. In some embodiments, a total number of hydrophobic amino acids in Y is more than 8, the total number of hydrophobic amino acids in Y is more than 12, the total number of hydrophobic amino acids in Y is more than 15, and/or a total number of hydrophilic amino acids in Y is no more than 5. In some embodiments, a total number of hydrophobic amino acids in Y is more than 8. In some embodiments, the total number of hydrophobic amino acids in Y is more than 12. In some embodiments, the total number of hydrophobic amino acids in Y is more than 15. In some embodiments, a total number of hydrophilic amino acids in Y is no more than 5. In some embodiments, Y comprises a sequence having at least 90% identity with any one selected from SEQ ID NOs: 3-15, 58-61 and 84-85, or Y comprises a sequence having at least 90% identity with any one selected from SEQ ID NOs: 3-18, 58-61 and 84-85. In some embodiments, Y comprises a sequence having at least 90% identity with any one selected from SEQ ID NOs: 3-15, 58-61 and 84-85. In some embodiments, Y comprises a sequence having at least 90% identity with any one selected from SEQ ID NOs: 3-18, 58-61 and 84-85.

In some embodiments, the allergic conjunctivitis is at least one selected from the group consisting of seasonal allergic conjunctivitis, perennial allergic conjunctivitis, vernal keratoconjunctivitis, giant papillary conjunctivitis, atopic keratoconjunctivitis and phlyctenular keratoconjunctivitis. In some embodiments, the seasonal allergic conjunctivitis is caused or partially caused by a seasonal allergen. In some embodiments, the seasonal allergen is at least one selected from the group consisting of tree pollens, weed pollens, grass pollens and mold spores. In some embodiments, the perennial allergic conjunctivitis is caused or partially caused by a perennial allergen. In some embodiments, the perennial allergen is at least one selected from the group consisting of dust mites, cockroaches and animal danders. In some embodiments, the vernal keratoconjunctivitis is at least one selected from the group consisting of palpebral vernal keratoconjunctivitis, limbal vernal keratoconjunctivitis and mixed vernal keratoconjunctivitis. In some embodiments, the giant papillary conjunctivitis is caused or partially caused by repeated mechanical irritation of the conjunctiva. In some embodiments, the repeated mechanical irritation of the conjunctiva is associated with at least one selected from the group consisting of contact lenses wearing, use of ocular prostheses, use of conjunctival sutures, use of extruding scleral buckles and elevated corneal scars. In some embodiments, the atopic keratoconjunctivitis is companied with atopic dermatitis and/or asthma. In some embodiments, the phlyctenular keratoconjunctivitis is caused or partially caused by a delayed hypersensitivity or inflammatory reaction of the cornea and conjunctiva to antigens expressed by various pathogens. In some embodiments, the pathogens are at least one selected from the group consisting of bacteria (e.g., Staphylococcus aureus, Streptococcus viridians, Mycobacterium tuberculosis), viruses (e.g., Herpes simplex virus), chlamydiae, fungi (e.g., Candida such as Candida albicans) and parasites (e.g., tapeworms such as Hymenolepis nana; and nematodes). In some embodiments, the subject does not have any ocular disease selected from the group consisting of viral conjunctivitis, bacterial conjunctivitis, chemical conjunctivitis and dry eye or dry eye associated disorders. In some embodiments, the subject experiences at least one symptom selected from the group consisting of redness of conjunctiva, edema of conjunctiva, hyperemia of conjunctiva, redness of eyelids, edema of eyelids, hyperemia of eyelids, ocular itching, visible damage of cornea, mucous discharge, watery discharge, tearing, photophobia, foreign body sensation and difficulty in opening eyes before administrating of the mutant of artificial polypeptide of formula (I). In some embodiments, the subject displays reduction in at least one symptom selected from the group consisting of redness of conjunctiva, edema of conjunctiva, hyperemia of conjunctiva, redness of eyelids, edema of eyelids, hyperemia of eyelids, ocular itching, visible damage of cornea, mucous discharge, watery discharge, tearing, photophobia, foreign body sensation and difficulty in opening eyes after administrating of at least one dose of the mutant of artificial polypeptide of formula (I). In some embodiments, the subject displays improvements in at least one tissue selected from the group consisting of conjunctiva, cornea, anterior chamber, iris, pupil, vitreum vitreous and fundus after administrating of at least one dose of the mutant of artificial polypeptide of formula (I).

In another aspect, provided is a method for treating or preventing allergic conjunctivitis in a subject in need thereof, comprising administering to the subject an effective amount of a mutant of artificial polypeptide of formula (II),

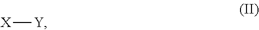

wherein X is a moiety comprising a sequence having at least 80% identity with SEQ ID NO: 1,
Y is a moiety comprising 10 to 50 amino acids, at least 50% of which are selected from I, V, L, F, C, M, and A,
Y comprises a sequence having at most 90% identity with SEQ ID NO: 2,
characterized in that the mutant has at least 1, 2, 3, 4, 5, 6, 7, 8, or 9 D in X mutated to S, and/or at least 1, 2, 3, 4, 5, or 6 E in X mutated to T.

In some embodiments, at least 20% amino acids of X are selected from R, K, N, D, Q, E, and H, at least 30% amino acids of X are selected from R, K, N, D, Q, E, and H, at most 90% amino acids of X are selected from R, K, N, D, Q, E, and H, and/or at most 80% amino acids of X are selected from R, K, N, D, Q, E, and H. In some embodiments, at least 20% amino acids of X are selected from R, K, N, D, Q, E, and H. In some embodiments, at least 30% amino acids of X are selected from R, K, N, D, Q, E, and H. In some embodiments, at most 90% amino acids of X are selected from R, K, N, D, Q, E, and H. In some embodiments, at most 80% amino acids of X are selected from R, K, N, D, Q, E, and H. In some embodiments, X comprises a sequence having at least 70% identity with SEQ ID NO: 1.

In some embodiments, Y comprises a sequence having at most 80% identity with SEQ ID NO: 2, or Y comprises a sequence having at most 70% identity with SEQ ID NO: 2. In some embodiments, Y comprises a sequence having at most 80% identity with SEQ ID NO: 2. In some embodiments, Y comprises a sequence having at most 70% identity with SEQ ID NO: 2. In some embodiments, a total number of hydrophobic amino acids in Y is more than 8, the total number of hydrophobic amino acids in Y is more than 12, the total number of hydrophobic amino acids in Y is more than 15, and/or a total number of hydrophilic amino acids in Y is no more than 5. In some embodiments, a total number of hydrophobic amino acids in Y is more than 8. In some embodiments, the total number of hydrophobic amino acids in Y is more than 12. In some embodiments, the total number of hydrophobic amino acids in Y is more than 15. In some embodiments, a total number of hydrophilic amino acids in Y is no more than 5. In some embodiments, Y comprises a sequence having at least 90% identity with any one selected from SEQ ID NOs: 16-18, or Y comprises a sequence having at least 95% identity with any one selected from SEQ ID NOs: 16-18. In some embodiments, Y comprises a sequence having at least 90% identity with any one selected from SEQ ID NOs: 16-18. In some embodiments, Y comprises a sequence having at least 95% identity with any one selected from SEQ ID NOs: 16-18.

In some embodiments, the allergic conjunctivitis is at least one selected from the group consisting of seasonal allergic conjunctivitis, perennial allergic conjunctivitis, vernal keratoconjunctivitis, giant papillary conjunctivitis, atopic keratoconjunctivitis and phlyctenular keratoconjunctivitis. In some embodiments, the seasonal allergic conjunctivitis is caused or partially caused by a seasonal allergen. In some embodiments, the seasonal allergen is at least one selected from the group consisting of tree pollens, weed pollens, grass pollens and mold spores. In some embodiments, the perennial allergic conjunctivitis is caused or partially caused by a perennial allergen. In some embodiments, the perennial allergen is at least one selected from the group consisting of dust mites, cockroaches and animal danders. In some embodiments, the vernal keratoconjunctivitis is at least one selected from the group consisting of palpebral vernal keratoconjunctivitis, limbal vernal keratoconjunctivitis and mixed vernal keratoconjunctivitis. In some embodiments, the giant papillary conjunctivitis is caused or partially caused by repeated mechanical irritation of the conjunctiva. In some embodiments, the repeated mechanical irritation of the conjunctiva is associated with at least one selected from the group consisting of contact lenses wearing, use of ocular prostheses, use of conjunctival sutures, use of extruding scleral buckles and elevated corneal scars. In some embodiments, the atopic keratoconjunctivitis is companied with atopic dermatitis and/or asthma. In some embodiments, the phlyctenular keratoconjunctivitis is caused or partially caused by a delayed hypersensitivity or inflammatory reaction of the cornea and conjunctiva to antigens expressed by various pathogens. In some embodiments, the pathogens are at least one selected from the group consisting of bacteria (e.g., Staphylococcus aureus, Streptococcus viridians, Mycobacterium tuberculosis), viruses (e.g., Herpes simplex virus), chlamydiae, fungi (e.g., Candida such as Candida albicans) and parasites (e.g., tapeworms such as Hymenolepis nana; and nematodes). In some embodiments, the subject does not have any ocular disease selected from the group consisting of viral conjunctivitis, bacterial conjunctivitis, chemical conjunctivitis and dry eye or dry eye associated disorders. In some embodiments, the subject experiences at least one symptom selected from the group consisting of redness of conjunctiva, edema of conjunctiva, hyperemia of conjunctiva, redness of eyelids, edema of eyelids, hyperemia of eyelids, ocular itching, visible damage of cornea, mucous discharge, watery discharge, tearing, photophobia, foreign body sensation and difficulty in opening eyes before administrating of the mutant of artificial polypeptide of formula (II). In some embodiments, the subject displays reduction in at least one symptom selected from the group consisting of redness of conjunctiva, edema of conjunctiva, hyperemia of conjunctiva, redness of eyelids, edema of eyelids, hyperemia of eyelids, ocular itching, visible damage of cornea, mucous discharge, watery discharge, tearing, photophobia, foreign body sensation and difficulty in opening eyes after administrating of at least one dose of the mutant of artificial polypeptide of formula (II). In some embodiments, the subject displays improvements in at least one tissue selected from the group consisting of conjunctiva, cornea, anterior chamber, iris, pupil, vitreum vitreous and fundus after administrating of at least one dose of the mutant of artificial polypeptide of formula (II).

In another aspect, provided is a method for treating or preventing allergic conjunctivitis in a subject in need thereof, comprising administering to the subject an effective amount of a mutant of artificial polypeptide of formula (III),

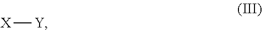

wherein X is a moiety comprising 40 to 65 amino acids, at least 50% of which are selected from H, R, K, D, Q, N and E,
Y is a moiety comprising a sequence having at least 80% identity with SEQ ID NO: 2,
a total number of H, R, K, D, Q, N and E in X is less than 33,
characterized in that the mutant has at least 1, 2, 3, 4, 5, 6, 7, 8, or 9 D in X mutated to S, and/or at least 1, 2, 3, 4, 5, or 6 E in X mutated to T.

In some embodiments, at least 20% amino acids of X are selected from R, K, N, D, Q, E, and H, at least 30% amino acids of X are selected from R, K, N, D, Q, E, and H, at most 90% amino acids of X are selected from R, K, N, D, Q, E, and H, and/or at most 80% amino acids of X are selected from R, K, N, D, Q, E, and H. In some embodiments, at least 20% amino acids of X are selected from R, K, N, D, Q, E, and H. In some embodiments, at least 30% amino acids of X are selected from R, K, N, D, Q, E, and H. In some embodiments, at most 90% amino acids of X are selected from R, K, N, D, Q, E, and H. In some embodiments, at most 80% amino acids of X are selected from R, K, N, D, Q, E, and H. In some embodiments, X comprises a sequence having at least 70% identity with SEQ ID NO: 1. In some embodiments, the total number of H, R, K, D, Q, N and E in X is less than 30, the total number of H, R, K, D, Q, N and E in X is less than 25, or the total number of H, R, K, D, Q, N and E in X is less than 20. In some embodiments, the total number of H, R, K, D, Q, N and E in X is less than 30. In some embodiments, the total number of H, R, K, D, Q, N and E in X is less than 25. In some embodiments, the total number of H, R, K, D, Q, N and E in X is less than 20. In some embodiments, a total number of hydrophilic amino acids in X is more than 10, a total number of hydrophilic amino acids in X is more than 15, a total number of hydrophilic amino acids in X is more than 20, a total number of hydrophilic amino acids in X is more than 25, a total number of hydrophobic amino acids in X is no more than 15, and/or the total number of hydrophobic amino acids in X is no more than 10. In some embodiments, a total number of hydrophilic amino acids in X is more than 10. In some embodiments, a total number of hydrophilic amino acids in X is more than 15. In some embodiments, a total number of hydrophilic amino acids in X is more than 20. In some embodiments, a total number of hydrophilic amino acids in X is more than 25. In some embodiments, a total number of hydrophobic amino acids in X is no more than 15. In some embodiments, the total number of hydrophobic amino acids in X is no more than 10.

In some embodiments, Y comprises a sequence having at least 90% identity with SEQ ID NO: 2, or Y comprises a sequence of SEQ ID NO: 2. In some embodiments, Y comprises a sequence having at least 90% identity with SEQ ID NO: 2. In some embodiments, Y comprises a sequence of SEQ ID NO: 2. In some embodiments, at least 50% amino acids of Y are selected from I, V, L, F, C, M, and A.

In some embodiments, the allergic conjunctivitis is at least one selected from the group consisting of seasonal allergic conjunctivitis, perennial allergic conjunctivitis, vernal keratoconjunctivitis, giant papillary conjunctivitis, atopic keratoconjunctivitis and phlyctenular keratoconjunctivitis. In some embodiments, the seasonal allergic conjunctivitis is caused or partially caused by a seasonal allergen. In some embodiments, the seasonal allergen is at least one selected from the group consisting of tree pollens, weed pollens, grass pollens and mold spores. In some embodiments, the perennial allergic conjunctivitis is caused or partially caused by a perennial allergen. In some embodiments, the perennial allergen is at least one selected from the group consisting of dust mites, cockroaches and animal danders. In some embodiments, the vernal keratoconjunctivitis is at least one selected from the group consisting of palpebral vernal keratoconjunctivitis, limbal vernal keratoconjunctivitis and mixed vernal keratoconjunctivitis. In some embodiments, the giant papillary conjunctivitis is caused or partially caused by repeated mechanical irritation of the conjunctiva. In some embodiments, the repeated mechanical irritation of the conjunctiva is associated with at least one selected from the group consisting of contact lenses wearing, use of ocular prostheses, use of conjunctival sutures, use of extruding scleral buckles and elevated corneal scars. In some embodiments, the atopic keratoconjunctivitis is companied with atopic dermatitis and/or asthma. In some embodiments, the phlyctenular keratoconjunctivitis is caused or partially caused by a delayed hypersensitivity or inflammatory reaction of the cornea and conjunctiva to antigens expressed by various pathogens. In some embodiments, the pathogens are at least one selected from the group consisting of bacteria (e.g., Staphylococcus aureus, Streptococcus viridians, Mycobacterium tuberculosis), viruses (e.g., Herpes simplex virus), chlamydiae, fungi (e.g., Candida such as Candida albicans) and parasites (e.g., tapeworms such as Hymenolepis nana; and nematodes). In some embodiments, the subject does not have any ocular disease selected from the group consisting of viral conjunctivitis, bacterial conjunctivitis, chemical conjunctivitis and dry eye or dry eye associated disorders. In some embodiments, the subject experiences at least one symptom selected from the group consisting of redness of conjunctiva, edema of conjunctiva, hyperemia of conjunctiva, redness of eyelids, edema of eyelids, hyperemia of eyelids, ocular itching, visible damage of cornea, mucous discharge, watery discharge, tearing, photophobia, foreign body sensation and difficulty in opening eyes before administrating of the mutant of artificial polypeptide of formula (III). In some embodiments, the subject displays reduction in at least one symptom selected from the group consisting of redness of conjunctiva, edema of conjunctiva, hyperemia of conjunctiva, redness of eyelids, edema of eyelids, hyperemia of eyelids, ocular itching, visible damage of cornea, mucous discharge, watery discharge, tearing, photophobia, foreign body sensation and difficulty in opening eyes after administrating of at least one dose of the mutant of artificial polypeptide of formula (III). In some embodiments, the subject displays improvements in at least one tissue selected from the group consisting of conjunctiva, cornea, anterior chamber, iris, pupil, vitreum vitreous and fundus after administrating of at least one dose of the mutant of artificial polypeptide of formula (III).

In another aspect, provided is a method for treating or preventing allergic conjunctivitis in a subject in need thereof, comprising administering to the subject an effective amount of a mutant of artificial polypeptide of formula (IV),

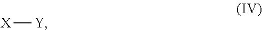

wherein X is a moiety comprising 40 to 65 amino acids, at least 50% of which are selected from H, R, K, D, Q, N and E,
Y is a moiety comprising a sequence having at least 80% identity with SEQ ID NO: 2,
X comprises a sequence having at most 90% identity with SEQ ID NO: 1,
characterized in that the mutant has at least 1, 2, 3, 4, 5, 6, 7, 8, or 9 D in X mutated to S, and/or at least 1, 2, 3, 4, 5, or 6 E in X mutated to T.

In some embodiments, at least 20% amino acids of X are selected from R, K, N, D, Q, E, and H, at least 30% amino acids of X are selected from R, K, N, D, Q, E, and H, at most 90% amino acids of X are selected from R, K, N, D, Q, E, and H, and/or at most 80% amino acids of X are selected from R, K, N, D, Q, E, and H. In some embodiments, at least 20% amino acids of X are selected from R, K, N, D, Q, E, and H. In some embodiments, at least 30% amino acids of X are selected from R, K, N, D, Q, E, and H. In some embodiments, at most 90% amino acids of X are selected from R, K, N, D, Q, E, and H. In some embodiments, at most 80% amino acids of X are selected from R, K, N, D, Q, E, and H. In some embodiments, X comprises a sequence having at least 70% identity with SEQ ID NO: 1. In some embodiments, a total number of hydrophilic amino acids in X is more than 10, a total number of hydrophilic amino acids in X is more than 15, a total number of hydrophilic amino acids in X is more than 20, a total number of hydrophilic amino acids in X is more than 25, a total number of hydrophobic amino acids in X is no more than 15, and/or the total number of hydrophobic amino acids in X is no more than 10. In some embodiments, a total number of hydrophilic amino acids in X is more than 10. In some embodiments, a total number of hydrophilic amino acids in X is more than 15. In some embodiments, a total number of hydrophilic amino acids in X is more than 20. In some embodiments, a total number of hydrophilic amino acids in X is more than 25. In some embodiments, a total number of hydrophobic amino acids in X is no more than 15. In some embodiments, the total number of hydrophobic amino acids in X is no more than 10.

In some embodiments, Y comprises a sequence having at least 90% identity with SEQ ID NO: 2, or Y comprises a sequence of SEQ ID NO: 2. In some embodiments, Y comprises a sequence having at least 90% identity with SEQ ID NO: 2. In some embodiments, Y comprises a sequence of SEQ ID NO: 2. In some embodiments, at least 50% amino acids of Y are selected from I, V, L, F, C, M, and A.

In some embodiments, the allergic conjunctivitis is at least one selected from the group consisting of seasonal allergic conjunctivitis, perennial allergic conjunctivitis, vernal keratoconjunctivitis, giant papillary conjunctivitis, atopic keratoconjunctivitis and phlyctenular keratoconjunctivitis. In some embodiments, the seasonal allergic conjunctivitis is caused or partially caused by a seasonal allergen. In some embodiments, the seasonal allergen is at least one selected from the group consisting of tree pollens, weed pollens, grass pollens and mold spores. In some embodiments, the perennial allergic conjunctivitis is caused or partially caused by a perennial allergen. In some embodiments, the perennial allergen is at least one selected from the group consisting of dust mites, cockroaches and animal danders. In some embodiments, the vernal keratoconjunctivitis is at least one selected from the group consisting of palpebral vernal keratoconjunctivitis, limbal vernal keratoconjunctivitis and mixed vernal keratoconjunctivitis. In some embodiments, the giant papillary conjunctivitis is caused or partially caused by repeated mechanical irritation of the conjunctiva. In some embodiments, the repeated mechanical irritation of the conjunctiva is associated with at least one selected from the group consisting of contact lenses wearing, use of ocular prostheses, use of conjunctival sutures, use of extruding scleral buckles and elevated corneal scars. In some embodiments, the atopic keratoconjunctivitis is companied with atopic dermatitis and/or asthma. In some embodiments, the phlyctenular keratoconjunctivitis is caused or partially caused by a delayed hypersensitivity or inflammatory reaction of the cornea and conjunctiva to antigens expressed by various pathogens. In some embodiments, the pathogens are at least one selected from the group consisting of bacteria (e.g., Staphylococcus aureus, Streptococcus viridians, Mycobacterium tuberculosis), viruses (e.g., Herpes simplex virus), chlamydiae, fungi (e.g., Candida such as Candida albicans) and parasites (e.g., tapeworms such as Hymenolepis nana; and nematodes). In some embodiments, the subject does not have any ocular disease selected from the group consisting of viral conjunctivitis, bacterial conjunctivitis, chemical conjunctivitis and dry eye or dry eye associated disorders. In some embodiments, the subject experiences at least one symptom selected from the group consisting of redness of conjunctiva, edema of conjunctiva, hyperemia of conjunctiva, redness of eyelids, edema of eyelids, hyperemia of eyelids, ocular itching, visible damage of cornea, mucous discharge, watery discharge, tearing, photophobia, foreign body sensation and difficulty in opening eyes before administrating of the mutant of artificial polypeptide of formula (IV). In some embodiments, the subject displays reduction in at least one symptom selected from the group consisting of redness of conjunctiva, edema of conjunctiva, hyperemia of conjunctiva, redness of eyelids, edema of eyelids, hyperemia of eyelids, ocular itching, visible damage of cornea, mucous discharge, watery discharge, tearing, photophobia, foreign body sensation and difficulty in opening eyes after administrating of at least one dose of the mutant of artificial polypeptide of formula (IV). In some embodiments, the subject displays improvements in at least one tissue selected from the group consisting of conjunctiva, cornea, anterior chamber, iris, pupil, vitreum vitreous and fundus after administrating of at least one dose of the mutant of artificial polypeptide of formula (IV).

In another aspect, provided is a method for treating or preventing allergic conjunctivitis in a subject in need thereof, comprising administering to the subject an effective amount of a mutant of artificial polypeptide of formula (V),

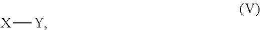

wherein X is a moiety comprising a sequence having at least 80% identity with SEQ ID NO: 1,
Y is a moiety comprising 10 to 30 amino acids, wherein Y comprises a sequence having at least 10 continuous AAs of SEQ ID NO: 2,
characterized in that the mutant has at least 1, 2, 3, 4, 5, 6, 7, 8, or 9 D in X mutated to S, and/or at least 1, 2, 3, 4, 5, or 6 E in X mutated to T.

In some embodiments, at least 20% amino acids of X are selected from R, K, N, D, Q, E, and H, at least 30% amino acids of X are selected from R, K, N, D, Q, E, and H, at most 90% amino acids of X are selected from R, K, N, D, Q, E, and H, and/or at most 80% amino acids of X are selected from R, K, N, D, Q, E, and H. In some embodiments, at least 20% amino acids of X are selected from R, K, N, D, Q, E, and H. In some embodiments, at least 30% amino acids of X are selected from R, K, N, D, Q, E, and H. In some embodiments, at most 90% amino acids of X are selected from R, K, N, D, Q, E, and H. In some embodiments, at most 80% amino acids of X are selected from R, K, N, D, Q, E, and H. In some embodiments, X comprises a sequence having at least 70% identity with SEQ ID NO: 1.

In some embodiments, Y comprises 10 to 25 amino acids, Y comprises 10 to 20 amino acids, or Y comprises 10 to 15 amino acids. In some embodiments, Y comprises 10 to 25 amino acids. In some embodiments, Y comprises 10 to 20 amino acids. In some embodiments, Y comprises 10 to 15 amino acids.

In some embodiments, Y comprises a sequence having at most 80% identity with SEQ ID NO: 2, Y comprises a sequence having at most 70% identity with SEQ ID NO: 2, or Y comprises a sequence having at most 50% identity with SEQ ID NO: 2. In some embodiments, Y comprises a sequence having at most 80% identity with SEQ ID NO: 2. In some embodiments, Y comprises a sequence having at most 70% identity with SEQ ID NO: 2. In some embodiments, Y comprises a sequence having at most 50% identity with SEQ ID NO: 2.

In some embodiments, the allergic conjunctivitis is at least one selected from the group consisting of seasonal allergic conjunctivitis, perennial allergic conjunctivitis, vernal keratoconjunctivitis, giant papillary conjunctivitis, atopic keratoconjunctivitis and phlyctenular keratoconjunctivitis. In some embodiments, the seasonal allergic conjunctivitis is caused or partially caused by a seasonal allergen. In some embodiments, the seasonal allergen is at least one selected from the group consisting of tree pollens, weed pollens, grass pollens and mold spores. In some embodiments, the perennial allergic conjunctivitis is caused or partially caused by a perennial allergen. In some embodiments, the perennial allergen is at least one selected from the group consisting of dust mites, cockroaches and animal danders. In some embodiments, the vernal keratoconjunctivitis is at least one selected from the group consisting of palpebral vernal keratoconjunctivitis, limbal vernal keratoconjunctivitis and mixed vernal keratoconjunctivitis. In some embodiments, the giant papillary conjunctivitis is caused or partially caused by repeated mechanical irritation of the conjunctiva. In some embodiments, the repeated mechanical irritation of the conjunctiva is associated with at least one selected from the group consisting of contact lenses wearing, use of ocular prostheses, use of conjunctival sutures, use of extruding scleral buckles and elevated corneal scars. In some embodiments, the atopic keratoconjunctivitis is companied with atopic dermatitis and/or asthma. In some embodiments, the phlyctenular keratoconjunctivitis is caused or partially caused by a delayed hypersensitivity or inflammatory reaction of the cornea and conjunctiva to antigens expressed by various pathogens. In some embodiments, the pathogens are at least one selected from the group consisting of bacteria (e.g., Staphylococcus aureus, Streptococcus viridians, Mycobacterium tuberculosis), viruses (e.g., Herpes simplex virus), chlamydiae, fungi (e.g., Candida such as Candida albicans) and parasites (e.g., tapeworms such as Hymenolepis nana; and nematodes). In some embodiments, the subject does not have any ocular disease selected from the group consisting of viral conjunctivitis, bacterial conjunctivitis, chemical conjunctivitis and dry eye or dry eye associated disorders. In some embodiments, the subject experiences at least one symptom selected from the group consisting of redness of conjunctiva, edema of conjunctiva, hyperemia of conjunctiva, redness of eyelids, edema of eyelids, hyperemia of eyelids, ocular itching, visible damage of cornea, mucous discharge, watery discharge, tearing, photophobia, foreign body sensation and difficulty in opening eyes before administrating of the mutant of artificial polypeptide of formula (V). In some embodiments, the subject displays reduction in at least one symptom selected from the group consisting of redness of conjunctiva, edema of conjunctiva, hyperemia of conjunctiva, redness of eyelids, edema of eyelids, hyperemia of eyelids, ocular itching, visible damage of cornea, mucous discharge, watery discharge, tearing, photophobia, foreign body sensation and difficulty in opening eyes after administrating of at least one dose of the mutant of artificial polypeptide of formula (V). In some embodiments, the subject displays improvements in at least one tissue selected from the group consisting of conjunctiva, cornea, anterior chamber, iris, pupil, vitreum vitreous and fundus after administrating of at least one dose of the mutant of artificial polypeptide of formula (V).

In another aspect, provided is a method for treating or preventing allergic conjunctivitis in a subject in need thereof, comprising administering to the subject an effective amount of a polypeptide having at least 70% identity with any one selected from SEQ ID NO: 28, SEQ ID NOs: 57 and 62-74 or a fragment or variant thereof.

In some embodiments, the polypeptide is a polypeptide of any one of SEQ ID NO: 28, SEQ ID NOs: 57 and 62-74 or a fragment or variant thereof, the polypeptide is a polypeptide of any one of SEQ ID NO: 28, SEQ ID NOs: 57 and 62-74, the polypeptide is a polypeptide of any one of SEQ ID NOs: 28 and 62-74, or the polypeptide is a polypeptide of SEQ ID NO: 28. In some embodiments, the polypeptide is a polypeptide of any one of SEQ ID NO: 28, SEQ ID NOs: 57 and 62-74 or a fragment or variant thereof. In some embodiments, the polypeptide is a polypeptide of any one of SEQ ID NO: 28, SEQ ID NOs: 57 and 62-74. In some embodiments, the polypeptide is a polypeptide of any one of SEQ ID NOs: 28 and 62-74. In some embodiments, the polypeptide is a polypeptide of SEQ ID NO: 28.

In some embodiments, the allergic conjunctivitis is at least one selected from the group consisting of seasonal allergic conjunctivitis, perennial allergic conjunctivitis, vernal keratoconjunctivitis, giant papillary conjunctivitis, atopic keratoconjunctivitis and phlyctenular keratoconjunctivitis. In some embodiments, the seasonal allergic conjunctivitis is caused or partially caused by a seasonal allergen. In some embodiments, the seasonal allergen is at least one selected from the group consisting of tree pollens, weed pollens, grass pollens and mold spores. In some embodiments, the perennial allergic conjunctivitis is caused or partially caused by a perennial allergen. In some embodiments, the perennial allergen is at least one selected from the group consisting of dust mites, cockroaches and animal danders. In some embodiments, the vernal keratoconjunctivitis is at least one selected from the group consisting of palpebral vernal keratoconjunctivitis, limbal vernal keratoconjunctivitis and mixed vernal keratoconjunctivitis. In some embodiments, the giant papillary conjunctivitis is caused or partially caused by repeated mechanical irritation of the conjunctiva. In some embodiments, the repeated mechanical irritation of the conjunctiva is associated with at least one selected from the group consisting of contact lenses wearing, use of ocular prostheses, use of conjunctival sutures, use of extruding scleral buckles and elevated corneal scars. In some embodiments, the atopic keratoconjunctivitis is companied with atopic dermatitis and/or asthma. In some embodiments, the phlyctenular keratoconjunctivitis is caused or partially caused by a delayed hypersensitivity or inflammatory reaction of the cornea and conjunctiva to antigens expressed by various pathogens. In some embodiments, the pathogens are at least one selected from the group consisting of bacteria (e.g., Staphylococcus aureus, Streptococcus viridians, Mycobacterium tuberculosis), viruses (e.g., Herpes simplex virus), chlamydiae, fungi (e.g., Candida such as Candida albicans) and parasites (e.g., tapeworms such as Hymenolepis nana; and nematodes). In some embodiments, the subject does not have any ocular disease selected from the group consisting of viral conjunctivitis, bacterial conjunctivitis, chemical conjunctivitis and dry eye or dry eye associated disorders. In some embodiments, the subject experiences at least one symptom selected from the group consisting of redness of conjunctiva, edema of conjunctiva, hyperemia of conjunctiva, redness of eyelids, edema of eyelids, hyperemia of eyelids, ocular itching, visible damage of cornea, mucous discharge, watery discharge, tearing, photophobia, foreign body sensation and difficulty in opening eyes before administrating of the polypeptide having at least 70% identity with any one selected from SEQ ID NO: 28, SEQ ID NOs: 57 and 62-74 or a fragment or variant thereof. In some embodiments, the subject displays reduction in at least one symptom selected from the group consisting of redness of conjunctiva, edema of conjunctiva, hyperemia of conjunctiva, redness of eyelids, edema of eyelids, hyperemia of eyelids, ocular itching, visible damage of cornea, mucous discharge, watery discharge, tearing, photophobia, foreign body sensation and difficulty in opening eyes after administrating of at least one dose of the polypeptide having at least 70% identity with any one selected from SEQ ID NO: 28, SEQ ID NOs: 57 and 62-74 or a fragment or variant thereof. In some embodiments, the subject displays improvements in at least one tissue selected from the group consisting of conjunctiva, cornea, anterior chamber, iris, pupil, vitreum vitreous and fundus after administrating of at least one dose of the polypeptide having at least 70% identity with any one selected from SEQ ID NO: 28, SEQ ID NOs: 57 and 62-74 or a fragment or variant thereof.

In another aspect, provided is an artificial polypeptide of formula (I), (II), (III), (IV), (V), (VI), or (VII), or a mutant of artificial polypeptide of (I), (II), (III), (IV) or (V), or a polypeptide having at least 70% identity with any one selected from SEQ ID NO. 28, SEQ ID NOs. 57 and 62-74 or a fragment or variant thereof, for use in treating or preventing allergic conjunctivitis in a subject in need thereof.

In another aspect, provided is use of an artificial polypeptide of formula (I), (II), (III), (IV), (V), (VI), or (VII), or a mutant of artificial polypeptide of (I), (II), (III), (IV) or (V), or a polypeptide having at least 70% identity with any one selected from SEQ ID NO. 28, SEQ ID NOs. 57 and 62-74 or a fragment or variant thereof for the preparation of a medicament for treating or preventing allergic conjunctivitis in a subject in need thereof.

In some embodiments, the allergic conjunctivitis is at least one selected from the group consisting of seasonal allergic conjunctivitis, perennial allergic conjunctivitis, vernal keratoconjunctivitis, giant papillary conjunctivitis, atopic keratoconjunctivitis and phlyctenular keratoconjunctivitis. In some embodiments, the seasonal allergic conjunctivitis is caused or partially caused by a seasonal allergen. In some embodiments, the seasonal allergen is at least one selected from the group consisting of tree pollens, weed pollens, grass pollens and mold spores. In some embodiments, the perennial allergic conjunctivitis is caused or partially caused by a perennial allergen. In some embodiments, the perennial allergen is at least one selected from the group consisting of dust mites, cockroaches and animal danders. In some embodiments, the vernal keratoconjunctivitis is at least one selected from the group consisting of palpebral vernal keratoconjunctivitis, limbal vernal keratoconjunctivitis and mixed vernal keratoconjunctivitis. In some embodiments, the giant papillary conjunctivitis is caused or partially caused by repeated mechanical irritation of the conjunctiva. In some embodiments, the repeated mechanical irritation of the conjunctiva is associated with at least one selected from the group consisting of contact lenses wearing, use of ocular prostheses, use of conjunctival sutures, use of extruding scleral buckles and elevated corneal scars. In some embodiments, the atopic keratoconjunctivitis is companied with atopic dermatitis and/or asthma. In some embodiments, the phlyctenular keratoconjunctivitis is caused or partially caused by a delayed hypersensitivity or inflammatory reaction of the cornea and conjunctiva to antigens expressed by various pathogens. In some embodiments, the pathogens are at least one selected from the group consisting of bacteria (e.g., Staphylococcus aureus, Streptococcus viridians, Mycobacterium tuberculosis), viruses (e.g., Herpes simplex virus), chlamydiae, fungi (e.g., Candida such as Candida albicans) and parasites (e.g., tapeworms such as Hymenolepis nana; and nematodes). In some embodiments, the subject does not have any ocular disease selected from the group consisting of viral conjunctivitis, bacterial conjunctivitis, chemical conjunctivitis and dry eye or dry eye associated disorders. In some embodiments, the subject experiences at least one symptom selected from the group consisting of redness of conjunctiva, edema of conjunctiva, hyperemia of conjunctiva, redness of eyelids, edema of eyelids, hyperemia of eyelids, ocular itching, visible damage of cornea, mucous discharge, watery discharge, tearing, photophobia, foreign body sensation and difficulty in opening eyes before administrating of an artificial polypeptide of formula (I), (II), (III), (IV), (V), (VI), or (VII), or a mutant of artificial polypeptide of (I), (II), (III), (IV) or (V), or a polypeptide having at least 70% identity with any one selected from SEQ ID NO. 28, SEQ ID NOs. 57 and 62-74 or a fragment or variant thereof. In some embodiments, the subject displays reduction in at least one symptom selected from the group consisting of redness of conjunctiva, edema of conjunctiva, hyperemia of conjunctiva, redness of eyelids, edema of eyelids, hyperemia of eyelids, ocular itching, visible damage of cornea, mucous discharge, watery discharge, tearing, photophobia, foreign body sensation and difficulty in opening eyes after administrating of at least one dose of an artificial polypeptide of formula (I), (II), (III), (IV), (V), (VI), or (VII), or a mutant of artificial polypeptide of (I), (II), (III), (IV) or (V), or a polypeptide having at least 70% identity with any one selected from SEQ ID NO. 28, SEQ ID NOs. 57 and 62-74 or a fragment or variant thereof. In some embodiments, the subject displays improvements in at least one tissue selected from the group consisting of conjunctiva, cornea, anterior chamber, iris, pupil, vitreum vitreous and fundus after administrating of at least one dose of an artificial polypeptide of formula (I), (II), (III), (IV), (V), (VI), or (VII), or a mutant of artificial polypeptide of (I), (II), (III), (IV) or (V), or a polypeptide having at least 70% identity with any one selected from SEQ ID NO. 28, SEQ ID NOs. 57 and 62-74 or a fragment or variant thereof.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Various features of this disclosure are set forth with particularity in the appended claims. A better understanding of the features and advantages of the present disclosure will be obtained by reference to the following detailed description that sets forth illustrative embodiments, in which the principles of the invention are utilized, and the accompanying drawings of which:

FIG. 1 shows the inflammation scores of the mice from respective groups on Day 14 measured according to Example 2.

FIG. 2 shows the scratching times of the mice from respective groups on Day 14 measured according to Example 2.

### DETAILED DESCRIPTION

### Definition

As used herein, the following terms have the meanings ascribed to them unless specified otherwise.

As used in the specification and claims, the singular forms “a,” “an,” and “the” include plural references unless the context clearly dictates otherwise. For example, the term “a cell” includes a plurality of cells, including mixtures thereof.

As used herein, the terms “comprise”, “include”, “contain” and variations thereof are intended to mean open-ended transitional phrases that do not exclude the possibilities of additional substances or methods. When such terms are used to describe a certain pharmaceutical composition, formulation, kit, use or method of the present disclosure, it also encompasses the situation that the pharmaceutical composition, formulation, kit, use or method consists of the recited substances or methods. For instance, the expression “the solvents comprise water” also includes the situation wherein the solvents are consisting of water, i.e., the solvents contain water exclusively. In the context of this disclosure, the term “consisting of” is intended to mean a close-ended transitional phrase, which excludes the possibilities of additional substances or methods.

As used herein, ranges as recited in this disclosure are intended to explicitly disclose each of the endpoints of the range and each integer included in the range, unless otherwise indicated. For example, “X is a moiety comprising 40 to 65 amino acids” means that the number of amino acids in the moiety of X can be 40, 41, 42, 43, 44, 45, 46, 47, 48, 49, 50, 51, 52, 53, 54, 55, 56, 57, 58, 59, 60, 61, 62, 63, 64 or 65. For another example, “Y is a moiety comprising 10 to 50 amino acids” means that that the number of amino acids in moiety of Y can be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50. Additionally, any sub-ranges consisting of these integers are intended to be included within the scope of this disclosure. Accordingly, “X is a moiety comprising 40 to 65 amino acids” is regarded as explicitly disclosing the sub-ranges such as “X is a moiety comprising 41 to 64 amino acids”, “X is a moiety comprising 42 to 63 amino acids”, “X is a moiety comprising 43 to 62 amino acids” . . . , etc.

The term “about” or “approximately” herein means within an acceptable error range of the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Where particular values are described in the application and claims, unless otherwise stated, the term “about” or “approximately” meaning within an acceptable error range for the particular value should be assumed.

The term “optional(ly)” means that the subsequently described event of circumstances may or may not occur, and that the description includes instances where the event or circumstance occurs and instances in which it does not.

The terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, or any other manipulation, such as conjugation with a labeling component.

The term “fragment” as used herein, when applied to a protein, refers to a truncated form of a native biologically active protein that may or may not retain at least a portion of the therapeutic and/or biological activity.

The term “variant” as used herein, when applied to a protein, refers to a protein with sequence homology to the native biologically active protein that retains at least a portion of the therapeutic and/or biological activity of the biologically active protein. For example, a variant protein may share at least 70%, 75%, 80%, 85%, 90%, 95%, 96%, 97%, 98% or 99% amino acid sequence identity as compared to the reference biologically active protein.

As used herein the term “amino acid (AA)” refers to either natural and/or unnatural or synthetic amino acids, including but not limited to glycine and both the D or L optical isomers, and amino acid analogs and peptidomimetics. Standard single or three letter codes are used to designate amino acids.

The term “natural L-amino acid” means the L optical isomer forms of glycine (G), proline (P), alanine (A), valine (V), leucine (L), isoleucine (I), methionine (M), cysteine (C), phenylalanine (F), tyrosine (Y), tryptophan (W), histidine (H), lysine (K), arginine (R), glutamine (Q), asparagine (N), glutamic acid (E), aspartic acid (D), serine (S), and threonine (T).

The terms “hydrophilic” and “hydrophobic” refer to the degree of affinity that a substance has with water. A hydrophilic substance has a strong affinity for water, tending to dissolve in, mix with, or be wetted by water, while a hydrophobic substance substantially lacks affinity for water, tending to repel and not absorb water and tending not to dissolve in or mix with or be wetted by water. Amino acids can be characterized based on their hydrophobicity. Examples of “hydrophilic amino acids” are arginine, lysine, threonine, alanine, asparagine, and glutamine. Of particular interest are the hydrophilic amino acids aspartate, glutamate, serine, and glycine. In some embodiments, “hydrophilic amino acids” refers to arginine, lysine, threonine, alanine, asparagine, glutamine, aspartate, glutamate, serine, and glycine.

Examples of “hydrophobic amino acids” are tryptophan, tyrosine, phenylalanine, methionine, leucine, isoleucine, and valine. In some embodiments, “hydrophobic amino acids” refers to tryptophan, tyrosine, phenylalanine, methionine, leucine, isoleucine, and valine.

A “host cell” includes an individual cell or cell culture which can be or has been a recipient for the subject vectors. Host cells include progeny of a single host cell. The progeny may not necessarily be completely identical (in morphology or in genomic of total DNA complement) to the original parent cell.

“Conjugated”, “linked,” “fused,” and “fusion” are used interchangeably herein. These terms refer to the joining together of two more chemical elements or components, by whatever means including chemical conjugation or recombinant means.

The terms “polynucleotides”, “nucleic acids”, “nucleotides” and “oligonucleotides” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “complement of a polynucleotide” denotes a polynucleotide molecule having a complementary base sequence and reverse orientation as compared to a reference sequence, such that it could hybridize with a reference sequence with complete fidelity.

The term “recombinant” as applied to a polynucleotide means that the polynucleotide is the product of various combinations of in vitro cloning, restriction and/or ligation steps, and other procedures that result in a construct that can potentially be expressed in a host cell.

The term “homology” or “homologous” refers to sequence similarity or interchangeability between two or more polynucleotide sequences or two or more polypeptide sequences. When using a program such as BestFit to determine sequence identity, similarity or homology between two different amino acid sequences, the default settings may be used, or an appropriate scoring matrix, such as blosum45 or blosum80, may be selected to optimize identity, similarity or homology scores. Preferably, polynucleotides that are homologous are those which hybridize under stringent conditions as defined herein and have at least 70%, preferably at least 80%, more preferably at least 90%, more preferably 95%, more preferably 97%, more preferably 98%, and even more preferably 99% sequence identity to those sequences.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity may be measured over the length of an entire defined polynucleotide sequence, for example, as defined by a particular SEQ ID number, or may be measured over a shorter length, for example, over the length of a fragment taken from a larger, defined polynucleotide sequence, for instance, a fragment of at least 45, at least 60, at least 90, at least 120, at least 150, at least 210 or at least 450 contiguous residues. Such lengths are exemplary only, and it is understood that any fragment length supported by the sequences shown herein, in the tables, figures or Sequence Listing, may be used to describe a length over which percentage identity may be measured.

“Percent (%) amino acid sequence identity,” with respect to the polypeptide sequences identified herein, is defined as the percentage of amino acid residues in a query sequence that are identical with the amino acid residues of a second, reference polypeptide sequence or a portion thereof, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

A “vector” is a nucleic acid molecule, preferably self-replicating in an appropriate host, which transfers an inserted nucleic acid molecule into and/or between host cells. The term includes vectors that function primarily for insertion of DNA or RNA into a cell, replication of vectors that function primarily for the replication of DNA or RNA, and expression vectors that function for transcription and/or translation of the DNA or RNA. Also included are vectors that provide more than one of the above functions. An “expression vector” is a polynucleotide which, when introduced into an appropriate host cell, can be transcribed and translated into a polypeptide(s). An “expression system” usually connotes a suitable host cell comprised of an expression vector that can function to yield a desired expression product.

The term “physiological conditions” refers to a set of conditions in a living host as well as in vitro conditions, including temperature, salt concentration, pH, that mimic those conditions of a living subject.

A host of physiologically relevant conditions for use in in vitro assays have been established. Generally, a physiological buffer contains a physiological concentration of salt and is adjusted to a neutral pH ranging from about 6.5 to about 7.8, and preferably from about 7.0 to about 7.5. A variety of physiological buffers is listed in Sambrook et al. (1989). Physiologically relevant temperature ranges from about 25° C. to about 38° C., and preferably from about 35° C. to about 37° C.

The term “antagonist” as used herein, includes any molecule that partially or fully blocks, inhibits, or neutralizes a biological activity of a native polypeptide disclosed herein. Methods for identifying antagonists of a polypeptide may comprise contacting a native polypeptide with a candidate antagonist molecule and measuring a detectable change in one or more biological activities normally associated with the native polypeptide. In the context of the present disclosure, antagonists may include proteins, nucleic acids, carbohydrates, antibodies or any other molecules that decrease the effect of a biologically active protein.

The term “agonist” is used in the broadest sense and includes any molecule that mimics a biological activity of a native polypeptide disclosed herein. Suitable agonist molecules specifically include agonist antibodies or antibody fragments, fragments or amino acid sequence variants of native polypeptides, peptides, small organic molecules, etc. Methods for identifying agonists of a native polypeptide may comprise contacting a native polypeptide with a candidate agonist molecule and measuring a detectable change in one or more biological activities normally associated with the native polypeptide.

The term “activity” for the purposes herein refers to an action or effect of a component of a fusion protein consistent with that of the corresponding native biologically active protein, wherein “biological activity” refers to an in vitro or in vivo biological function or effect, including but not limited to receptor binding, antagonist activity, agonist activity, or a cellular or physiologic response.

As used herein, “treatment” or “treating,” “palliating,” and “ameliorating” are used interchangeably.

These terms refer to an approach for obtaining beneficial or desired results including but not limited to a therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disease condition such that an improvement is observed in the subject, notwithstanding that the subject may still be afflicted with the underlying disorder. For prophylactic benefit, the compositions may be administered to a subject at risk of developing a particular disease condition, or to a subject reporting one or more of the physiological symptoms of a disease, even though a diagnosis of this disease may not have been made.

The term “therapeutic effect” refers to a physiologic effect, including but not limited to the cure, mitigation, amelioration, or prevention of disease condition in humans or other animals, or to otherwise enhance physical or mental wellbeing of humans or animals, caused by a polypeptide of the present disclosure. Determination of a therapeutically effective amount is well within the capability of those skilled in the art, especially in light of the detailed disclosure provided herein.

The term “effective amount” refers to an amount of a biologically active protein, either alone or as a part of a fusion protein composition, that is capable of having any detectable, beneficial effect on any symptom, aspect, measured parameter or characteristics of a disease state or condition when administered in one or repeated doses to a subject. Such effect need not be absolute to be beneficial. The disease condition can refer to a disorder or a disease.

The term “pharmaceutically acceptable” refers to those compounds, materials, compositions, formulations or dosage forms which are, within the scope of sound medical judgment, suitable for use in contact with the tissues of human beings and other animals without excessive toxicity, irritation, allergic response, or other problem or complication, commensurate with a reasonable benefit/risk ratio.

The terms “formulation” and “dosage form” are used interchangeably and refer to a pharmaceutical composition that is formulated in accordance with clinical requirements, in a form that can be directly administered to a subject in need thereof for preventive or therapeutic use.

The term “pharmaceutically acceptable excipient” refers to a pharmaceutically acceptable material, carrier, or vehicle which are used for the preparation of the formulations or dosage forms in accordance with the present disclosure. Each excipient must be “acceptable” in the sense of being compatible with the other ingredients of the formulations or dosage forms and not injurious to the patient.

The term “subject” “individual” or “patient” as used herein refers to any animals that can be used in the present disclosure, including but not limited to human, primate, rodent, canine, feline, equine, ovine, porcine, and the like.

The terms “administer”, “administered”, “administers”, “administering” and “dosing” are used interchangeably and are defined as providing a compound, a composition, a formulation and/or a dosage form in accordance with the present disclosure to a subject in need thereof via a route known in the art, including but not limited to oral, buccal, topical, transmucosal, transdermal, rectal, and parenteral routes of administration. In some embodiments, an oral route of administration is used. In some embodiments, a parenteral route of administration, including intravenous, intraarterial, intramuscular, subcutaneous, intraosseous, and intraperitoneal is used. In some embodiments, a topical route of administration is used.

The term “in vivo” as used herein refers to an event that takes place in a subject's body.

The term “in vitro” as used herein refers to an event that takes places outside of a subject's body.

In some embodiments, an in vitro assay encompasses any assay run outside of a subject assay. In vitro assays encompass cell-based assays in which cells alive or dead are employed. In vitro assays also encompass a cell-free assay in which no intact cells are employed.

The term “allergic conjunctivitis” as used herein refers to a class of inflammatory diseases caused by hypersensitivity of conjunctiva to the stimulations of various allergens. Some common signs and symptoms of allergic conjunctivitis are, among others, redness, edema or hyperemia of conjutctiva, redness, edema or hyperemia of eyelids, ocular itching, visible damage of cornea, mucous discharge, watery discharge, tearing, photophobia, foreign body sensation and difficulty in opening eyes. It is also reported that patients may experience some other conditions such as itchy nose, sneezing, coughing, briefly blurred vision, distraction in mind, decreased productivity and tiredness.

The term “seasonal allergic conjunctivitis” is a most common form of allergic conjunctivitis, which is associated with seasonal allergies that usually occur during the spring and summer months, and sometimes during the fall. The seasonal allergic conjunctivitis is believed to be caused or partially caused by a seasonal allergen, such as tree pollens, weed pollens, grass pollens and mold spores.

The term “perennial allergic conjunctivitis” refers to a mild and chronic form of allergic conjunctivitis that is persists throughout the year. The perennial allergic conjunctivitis is believed to be caused or partially caused by a perennial allergen, i.e., a year-round environmental (usually indoor) allergen such as dust mites, cockroaches and animal danders.

The term “vernal keratoconjunctivitis” refers to a recurrent, bilateral, and self-limiting form of allergic conjunctivitis having a periodic seasonal incidence. The vernal keratoconjunctivitis is further classified into 3 subtypes clinically, i.e., palpebral vernal keratoconjunctivitis, limbal vernal keratoconjunctivitis and mixed vernal keratoconjunctivitis. The vernal keratoconjunctivitis is believed to be caused or partially caused by dust mites, pollens and/or animal danders. However, it is often hard to find allergen for most patients.

The term “giant papillary conjunctivitis” refers to a form of allergic conjunctivitis that is caused or partially caused by repeated mechanical irritation of the conjunctiva. For example, the repeated mechanical irritation of the conjunctiva may be associated with contact lenses wearing, use of ocular prostheses, use of conjunctival sutures, use of extruding scleral buckles and elevated corneal scars.

The term “atopic keratoconjunctivitis” refer to a form of chronic (long-lasting) allergic conjunctivitis which affects patients suffered by the skin condition of atopic dermatitis. The atopic keratoconjunctivitis is often companied with atopic dermatitis and/or asthma.

The term “phlyctenular keratoconjunctivitis” refer to a form of allergic conjunctivitis caused or partially caused by a delayed hypersensitivity or inflammatory reaction of the cornea and conjunctiva to antigens expressed by various pathogens. The triggering antigen is usually a bacterial protein (particularly from Staphylococcus aureus, Streptococcus viridians, Mycobacterium tuberculosis, etc.), but may also be a component (e.g., proteins) of viruses (e.g., Herpes simplex virus), chlamydiae, fungi (particularly Candida such as Candida albicans), or parasites (e.g., tapeworms such as Hymenolepis nana; and nematodes).

### Methods of Treatment and Therapeutic Uses

In one aspect, provided is a method for treating or preventing allergic conjunctivitis in a subject in need thereof, comprising administering to the subject an effective amount of an artificial polypeptide of formula (I):

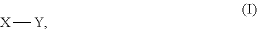

wherein X is a moiety comprising a sequence having at least 80% identity with SEQ ID NO: 1, and
Y is a moiety comprising 10 to 50 amino acids, at least 50% of which are selected from I, V, L, F, C, M, and A;
wherein Y comprises a total number of Cysteine (C) of less than 5.

In some embodiments, X of formula (I) can be a hydrophilic moiety, at least 50% of which are selected from R, K, N, D, Q, E, and H. In some embodiments, X of formula (I) can be a hydrophilic moiety, at least 55% of which are selected from R, K, N, D, Q, E, and H. In some embodiments, X of formula (I) can be a hydrophilic moiety, at least 60% of which are selected from R, K, N, D, Q, E, and H. In some embodiments, X of formula (I) can be a hydrophilic moiety, at least 65% of which are selected from R, K, N, D, Q, E, and H. In some embodiments, X of formula (I) can be a hydrophilic moiety, at least 70% of which are selected from R, K, N, D, Q, E, and H. In some embodiments, X of formula (I) can be a hydrophilic moiety, at least 75% of which are selected from R, K, N, D, Q, E, and H. In some embodiments, X of formula (I) can be a hydrophilic moiety, at least 80% of which are selected from R, K, N, D, Q, E, and H. In some embodiments, X of formula (I) can be a hydrophilic moiety, at least 85% of which are selected from R, K, N, D, Q, E, and H. In some embodiments, X of formula (I) can be a hydrophilic moiety, at least 90% of which are selected from R, K, N, D, Q, E, and H.

In some embodiments, X of formula (I) can be a moiety comprising a sequence having at least 80% identity with SEQ ID NO: 1. In some embodiments, X is a moiety comprising a sequence having at least 85% identity with SEQ ID NO: 1. In some embodiments, X is a moiety comprising a sequence having at least 90% identity with SEQ ID NO: 1. In some embodiments, X is a moiety comprising a sequence having at least 95% identity with SEQ ID NO: 1. In some embodiments, X is a moiety comprising a sequence having at least 96%, at least 97%, at least 98%, or at least 99% identity with SEQ ID NO: 1. In some embodiments, X is a moiety comprising the sequence of SEQ ID NO: 1. In some embodiments, X is a moiety that is the sequence of SEQ ID NO: 1.

In some embodiments, Y of formula (I) can be a hydrophobic moiety, at least 50% of which are selected from I, V, L, F, C, M, and A. In some embodiments, Y of formula (I) can be a hydrophobic moiety, at least 55% of which are selected from I, V, L, F, C, M, and A. In some embodiments, Y of formula (I) can be a hydrophobic moiety, at least 60% of which are selected from I, V, L, F, C, M, and A. In some embodiments, Y of formula (I) can be a hydrophobic moiety, at least 65% of which are selected from I, V, L, F, C, M, and A. In some embodiments, Y of formula (I) can be a hydrophobic moiety, at least 70% of which are selected from I, V, L, F, C, M, and A. In some embodiments, Y of formula (I) can be a hydrophobic moiety, at least 75% of which are selected from I, V, L, F, C, M, and A. In some embodiments, Y of formula (I) can be a hydrophobic moiety, at least 80% of which are selected from I, V, L, F, C, M, and A. In some embodiments, Y of formula (I) can be a hydrophobic moiety, at least 85% of which are selected from I, V, L, F, C, M, and A. In some embodiments, Y of formula (I) can be a hydrophobic moiety, at least 90% of which are selected from I, V, L, F, C, M, and A.

In some embodiments, Y of formula (I) can be a moiety comprising 10 to 50 amino acids, and a total number of hydrophobic amino acids of Y is 5. In some embodiments, Y of formula (I) can be a moiety comprising 10 to 50 amino acids, and a total number of hydrophobic amino acids of Y is more than 5. In some embodiments, Y of formula (I) can be a moiety comprising 10 to 50 amino acids, and a total number of hydrophobic amino acids of Y is 6. In some embodiments, Y of formula (I) can be a moiety comprising 10 to 50 amino acids, and a total number of hydrophobic amino acids of Y is more than 6. In some embodiments, Y of formula (I) can be a moiety comprising 10 to 50 amino acids, and a total number of hydrophobic amino acids of Y is 7. In some embodiments, Y of formula (I) can be a moiety comprising 10 to 50 amino acids, and a total number of hydrophobic amino acids of Y is more than 7. In some embodiments, Y of formula (I) can be a moiety comprising 10 to 50 amino acids, and a total number of hydrophobic amino acids of Y is 8. In some embodiments, Y of formula (I) can be a moiety comprising 10 to 50 amino acids, and a total number of hydrophobic amino acids of Y is more than 8. In some embodiments, Y of formula (I) can be a moiety comprising 10 to 50 amino acids, and a total number of hydrophobic amino acids of Y is 9. In some embodiments, Y of formula (I) can be a moiety comprising 10 to 50 amino acids, and a total number of hydrophobic amino acids of Y is more than 9. In some embodiments, Y of formula (I) can be a moiety comprising 10 to 50 amino acids, and a total number of hydrophobic amino acids of Y is 10. In some embodiments, Y of formula (I) can be a moiety comprising 10 to 50 amino acids, and a total number of hydrophobic amino acids of Y is more than 10. In some embodiments, Y of formula (I) can be a moiety comprising 10 to 50 amino acids, and a total number of hydrophobic amino acids of Y is 11. In some embodiments, Y of formula (I) can be a moiety comprising 10 to 50 amino acids, and a total number of hydrophobic amino acids of Y is more than 11. In some embodiments, Y of formula (I) can be a moiety comprising 10 to 50 amino acids, and a total number of hydrophobic amino acids of Y is 12. In some embodiments, Y of formula (I) can be a moiety comprising 10 to 50 amino acids, and a total number of hydrophobic amino acids of Y is more than 12. In some embodiments, Y of formula (I) can be a moiety comprising 10 to 50 amino acids, and a total number of hydrophobic amino acids of Y is 13. In some embodiments, Y of formula (I) can be a moiety comprising 10 to 50 amino acids, and a total number of hydrophobic amino acids of Y is more than 13. In some embodiments, Y of formula (I) can be a moiety comprising 10 to 50 amino acids, and a total number of hydrophobic amino acids of Y is 14. In some embodiments, Y of formula (I) can be a moiety comprising 10 to 50 amino acids, and a total number of hydrophobic amino acids of Y is more than 14. In some embodiments, Y of formula (I) can be a moiety comprising 10 to 50 amino acids, and a total number of hydrophobic amino acids of Y is 15. In some embodiments, Y of formula (I) can be a moiety comprising 10 to 50 amino acids, and a total number of hydrophobic amino acids of Y is more than 15.

In some embodiments, Y of formula (I) can be a moiety comprising 10 to 50 amino acids, and a total number of I, V, L, F, C, M, and A of Y is 5. In some embodiments, Y of formula (I) can be a moiety comprising 10 to 50 amino acids, and a total number of I, V, L, F, C, M, and A of Y is more than 5. In some embodiments, Y of formula (I) can be a moiety comprising 10 to 50 amino acids, and a total number of I, V, L, F, C, M, and A of Y is 6. In some embodiments, Y of formula (I) can be a moiety comprising 10 to 50 amino acids, and a total number of I, V, L, F, C, M, and A of Y is more than 6. In some embodiments, Y of formula (I) can be a moiety comprising 10 to 50 amino acids, and a total number of I, V, L, F, C, M, and A of Y is 7. In some embodiments, Y of formula (I) can be a moiety comprising 10 to 50 amino acids, and a total number of I, V, L, F, C, M, and A of Y is more than 7. In some embodiments, Y of formula (I) can be a moiety comprising 10 to 50 amino acids, and a total number of I, V, L, F, C, M, and A of Y is 8.

In some embodiments, Y of formula (I) can be a moiety comprising 10 to 50 amino acids, and a total number of I, V, L, F, C, M, and A of Y is more than 8. In some embodiments, Y of formula (I) can be a moiety comprising 10 to 50 amino acids, and a total number of I, V, L, F, C, M, and A of Y is 9. In some embodiments, Y of formula (I) can be a moiety comprising 10 to 50 amino acids, and a total number of I, V, L, F, C, M, and A of Y is more than 9. In some embodiments, Y of formula (I) can be a moiety comprising 10 to 50 amino acids, and a total number of I, V, L, F, C, M, and A of Y is 10. In some embodiments, Y of formula (I) can be a moiety comprising 10 to 50 amino acids, and a total number of I, V, L, F, C, M, and A of Y is more than 10. In some embodiments, Y of formula (I) can be a moiety comprising 10 to 50 amino acids, and a total number of I, V, L, F, C, M, and A of Y is 11. In some embodiments, Y of formula (I) can be a moiety comprising 10 to 50 amino acids, and a total number of I, V, L, F, C, M, and A of Y is more than 11. In some embodiments, Y of formula (I) can be a moiety comprising 10 to 50 amino acids, and a total number of I, V, L, F, C, M, and A of Y is 12. In some embodiments, Y of formula (I) can be a moiety comprising 10 to 50 amino acids, and a total number of I, V, L, F, C, M, and A of Y is more than 12. In some embodiments, Y of formula (I) can be a moiety comprising 10 to 50 amino acids, and a total number of I, V, L, F, C, M, and A of Y is 13. In some embodiments, Y of formula (I) can be a moiety comprising 10 to 50 amino acids, and a total number of I, V, L, F, C, M, and A of Y is more than 13. In some embodiments, Y of formula (I) can be a moiety comprising 10 to 50 amino acids, and a total number of I, V, L, F, C, M, and A of Y is 14. In some embodiments, Y of formula (I) can be a moiety comprising 10 to 50 amino acids, and a total number of I, V, L, F, C, M, and A of Y is more than 14. In some embodiments, Y of formula (I) can be a moiety comprising 10 to 50 amino acids, and a total number of I, V, L, F, C, M, and A of Y is 15. In some embodiments, Y of formula (I) can be a moiety comprising 10 to 50 amino acids, and a total number of I, V, L, F, C, M, and A of Y is more than 15.

In some embodiments, Y of formula (I) can be a moiety comprising 10 to 50 amino acids, and a total number of Cysteine (C) of Y is 5. In some embodiments, Y of formula (I) can be a moiety comprising 10 to 50 amino acids, and a total number of Cysteine (C) of Y is less than 5. In some embodiments, Y of formula (I) can be a moiety comprising 10 to 50 amino acids, and a total number of Cysteine (C) of Y is 4.

In some embodiments, Y of formula (I) can be a moiety comprising 10 to 50 amino acids, and a total number of Cysteine (C) of Y is less than 4. In some embodiments, Y of formula (I) can be a moiety comprising 10 to 50 amino acids, and a total number of Cysteine (C) of Y is 3. In some embodiments, Y of formula (I) can be a moiety comprising 10 to 50 amino acids, and a total number of Cysteine (C) of Y is less than 3. In some embodiments, Y of formula (I) can be a moiety comprising 10 to 50 amino acids, and a total number of Cysteine (C) of Y is 2. In some embodiments, Y of formula (I) can be a moiety comprising 10 to 50 amino acids, and a total number of Cysteine (C) of Y is less than 2. In some embodiments, Y of formula (I) can be a moiety comprising 10 to 50 amino acids, and a total number of Cysteine (C) of Y is 1. In some embodiments, Y of formula (I) can be a moiety comprising 10 to 50 amino acids, and a total number of Cysteine (C) of Y is less than 1. In some embodiments, Y of formula (I) can be a moiety comprising 10 to 50 amino acids, and a total number of Cysteine (C) of Y is 0.

In some embodiments, a total number of hydrophilic amino acids in Y of formula (I) is 5. In some embodiments, a total number of hydrophilic amino acids in Y of formula (I) is no more than 5. In some embodiments, a total number of hydrophilic amino acids in Y of formula (I) is 4. In some embodiments, a total number of hydrophilic amino acids in Y of formula (I) is no more than 4. In some embodiments, a total number of hydrophilic amino acids in Y of formula (I) is 3. In some embodiments, a total number of hydrophilic amino acids in Y of formula (I) is no more than 3. In some embodiments, a total number of hydrophilic amino acids in Y of formula (I) is 2. In some embodiments, a total number of hydrophilic amino acids in Y of formula (I) is no more than 2. In some embodiments, a total number of hydrophilic amino acids in Y of formula (I) is 1. In some embodiments, a total number of hydrophilic amino acids in Y of formula (I) is no more than 1.

In some embodiments, a total number of R, K, N, D, Q, E, and H in Y of formula (I) is 5. In some embodiments, a total number of R, K, N, D, Q, E, and H in Y of formula (I) is no more than 5. In some embodiments, a total number of hydrophilic amino acids in Y of formula (I) is 4. In some embodiments, a total number of R, K, N, D, Q, E, and H in Y of formula (I) is no more than 4. In some embodiments, a total number of R, K, N, D, Q, E, and H in Y of formula (I) is 3. In some embodiments, a total number of R, K, N, D, Q, E, and H in Y of formula (I) is no more than 3. In some embodiments, a total number of R, K, N, D, Q, E, and H in Y of formula (I) is 2. In some embodiments, a total number of R, K, N, D, Q, E, and H in Y of formula (I) is no more than 2. In some embodiments, a total number of R, K, N, D, Q, E, and H in Y of formula (I) is 1. In some embodiments, a total number of R, K, N, D, Q, E, and H in Y of formula (I) is no more than 1.

In some embodiments of the artificial polypeptide, X of formula (I) comprises a sequence having at least 70% identity with any one selected from SEQ ID NOs: 81-83. In some embodiments of the artificial polypeptide, X of formula (I) comprises a sequence having at least 75% identity with any one selected from SEQ ID NOs: 81-83. In some embodiments of the artificial polypeptide, X of formula (I) comprises a sequence having at least 80% identity with any one selected from SEQ ID NOs: 81-83. In some embodiments of the artificial polypeptide, X of formula (I) comprises a sequence having at least 85% identity with any one selected from SEQ ID NOs: 81-83. In some embodiments of the artificial polypeptide, X of formula (I) comprises a sequence having at least 90% identity with any one selected from SEQ ID NOs: 81-83. In some embodiments of the artificial polypeptide, X of formula (I) comprises a sequence having at least 95% identity with any one selected from SEQ ID NOs: 81-83. In some embodiments of the artificial polypeptide, X of formula (I) comprises a sequence having at least 96%, at least 97%, at least 98%, or at least 99% identity with any one selected from SEQ ID NOs: 81-83. In some embodiments of the artificial polypeptide, X of formula (I) comprises a sequence of any one selected from SEQ ID NOs: 81-83.

In some embodiments of the artificial polypeptide, X of formula (I) is a sequence of any one selected from SEQ ID NOs: 81-83.

In some embodiments of the artificial polypeptide, Y of formula (I) comprises a sequence having at least 70% identity with any one selected from SEQ ID NOs: 3-15, 58-61 and 84-85. In some embodiments of the artificial polypeptide, Y of formula (I) comprises a sequence having at least 75% identity with any one selected from SEQ ID NOs: 3-15, 58-61 and 84-85. In some embodiments of the artificial polypeptide, Y of formula (I) comprises a sequence having at least 80% identity with any one selected from SEQ ID NOs: 3-15, 58-61 and 84-85. In some embodiments of the artificial polypeptide, Y of formula (I) comprises a sequence having at least 85% identity with any one selected from SEQ ID NOs: 3-15, 58-61 and 84-85.

In some embodiments of the artificial polypeptide, Y of formula (I) comprises a sequence having at least 90% identity with any one selected from SEQ ID NOs: 3-15, 58-61 and 84-85. In some embodiments of the artificial polypeptide, Y of formula (I) comprises a sequence having at least 95% identity with any one selected from SEQ ID NOs: 3-15, 58-61 and 84-85. In some embodiments of the artificial polypeptide, Y of formula (I) comprises a sequence having at least 96%, at least 97%, at least 98%, or at least 99% identity with any one selected from SEQ ID NOs: 3-15, 58-61 and 84-85. In some embodiments of the artificial polypeptide, Y of formula (I) comprises a sequence of any one selected from SEQ ID NOs: 3-15, 58-61 and 84-85. In some embodiments of the artificial polypeptide, Y of formula (I) is a sequence of any one selected from SEQ ID NOs: 3-15, 58-61 and 84-85.

In some embodiments of the artificial polypeptide of formula (I), the polypeptide comprises a sequence having at least 70% identity with any one selected from SEQ ID NOs: 29-41, 54-57, 75 and 91-95. In some embodiments of the artificial polypeptide of formula (I), the polypeptide comprises a sequence having at least 75% identity with any one selected from SEQ ID NOs: 29-41, 54-57, 75 and 91-95. In some embodiments of the artificial polypeptide of formula (I), the polypeptide comprises a sequence having at least 80% identity with any one selected from SEQ ID NOs: 29-41, 54-57, 75 and 91-95. In some embodiments of the artificial polypeptide of formula (I), the polypeptide comprises a sequence having at least 85% identity with any one selected from SEQ ID NOs: 29-41, 54-57, 75 and 91-95. In some embodiments of the artificial polypeptide of formula (I), the polypeptide comprises a sequence having at least 90% identity with any one selected from SEQ ID NOs: 29-41, 54-57, 75 and 91-95. In some embodiments of the artificial polypeptide of formula (I), the polypeptide comprises a sequence having at least 95% identity with any one selected from SEQ ID NOs: 29-41, 54-57, 75 and 91-95. In some embodiments of the artificial polypeptide of formula (I), the polypeptide comprises a sequence having at least 96%, at least 97%, at least 98%, or at least 99% identity with any one selected from SEQ ID NOs: 29-41, 54-57, 75 and 91-95. In some embodiments of the artificial polypeptide of formula (I), the polypeptide comprises a sequence of any one of SEQ ID NOs: 29-41, 54-57, 75 and 91-95. In some embodiments of the artificial polypeptide of formula (I), the polypeptide is a sequence of any one of SEQ ID NOs: 29-41, 54-57, 75 and 91-95.

In another aspect, provided is a method for treating or preventing allergic conjunctivitis in a subject in need thereof, comprising administering to the subject an effective amount of an artificial polypeptide of formula (II):

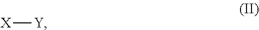

wherein X is a moiety comprising a sequence having at least 80% identity with SEQ ID NO: 1, and
Y is a moiety comprising 10 to 50 amino acids, at least 50% of which are selected from I, V, L, F, C, M, and A;
wherein Y comprises a sequence having at most 90% identity with SEQ ID NO: 2.

In some embodiments, X of formula (II) can be a hydrophilic moiety, at least 50% of which are selected from R, K, N, D, Q, E, and H. In some embodiments, X of formula (II) can be a hydrophilic moiety, at least 55% of which are selected from R, K, N, D, Q, E, and H. In some embodiments, X of formula (I) can be a hydrophilic moiety, at least 60% of which are selected from R, K, N, D, Q, E, and H. In some embodiments, X of formula (II) can be a hydrophilic moiety, at least 65% of which are selected from R, K, N, D, Q, E, and H. In some embodiments, X of formula (II) can be a hydrophilic moiety, at least 70% of which are selected from R, K, N, D, Q, E, and H. In some embodiments, X of (II) can be a hydrophilic moiety, at least 75% of which are selected from R, K, N, D, Q, E, and H. In some embodiments, X of formula (II) can be a hydrophilic moiety, at least 80% of which are selected from R, K, N, D, Q, E, and H.

In some embodiments, X of formula (II) can be a hydrophilic moiety, at least 85% of which are selected from R, K, N, D, Q, E, and H. In some embodiments, X of formula (II) can be a hydrophilic moiety, at least 90% of which are selected from R, K, N, D, Q, E, and H.

In some embodiments, X of formula (II) can be a hydrophilic moiety comprising one or more Arginine (R). For example, X of formula (II) can be a hydrophilic moiety comprising at least one, at least two, at least three, at least four, at least five, at least six, at least seven, at least eight, at least nine, or at least ten Arginine (R). In some embodiments, X of formula (II) can be a hydrophilic moiety comprising one or more Lysine (K). For example, X of formula (II) can be a hydrophilic moiety comprising at least one, at least two, at least three, at least four, at least five, at least six, at least seven, at least eight, at least nine, or at least ten Lysine (K). In some embodiments, X of formula (II) can be a hydrophilic moiety comprising one or more Asparagine (N). For example, X of formula (II) can be a hydrophilic moiety comprising at least one, at least two, at least three, at least four, at least five, at least six, at least seven, at least eight, at least nine, or at least ten Asparagine (N). In some embodiments, X of formula (II) can be a hydrophilic moiety comprising one or more Aspartic Acid (D). For example, X of formula (II) can be a hydrophilic moiety comprising at least one, at least two, at least three, at least four, at least five, at least six, at least seven, at least eight, at least nine, or at least ten Aspartic Acid (D). In some embodiments, X of formula (II) can be a hydrophilic moiety comprising one or more Glutamine (Q). For example, X of formula (II) can be a hydrophilic moiety comprising at least one, at least two, at least three, at least four, at least five, at least six, at least seven, at least eight, at least nine, or at least ten Glutamine (Q). In some embodiments, X of formula (II) can be a hydrophilic moiety comprising one or more Glutamic Acid (E). For example, X of formula (II) can be a hydrophilic moiety comprising at least one, at least two, at least three, at least four, at least five, at least six, at least seven, at least eight, at least nine, or at least ten Glutamic Acid (E). In some embodiments, X of formula (II) can be a hydrophilic moiety comprising one or more Histidine (H). For example, X of formula (II) can be a hydrophilic moiety comprising at least one, at least two, at least three, at least four, at least five, at least six, at least seven, at least eight, at least nine, or at least ten Histidine (H).

In some embodiments, X of formula (II) can be a moiety comprising a sequence having at least 80% identity with SEQ ID NO: 1. In some embodiments, X is a moiety comprising a sequence having at least 85% identity with SEQ ID NO: 1. In some embodiments, X is a moiety comprising a sequence having at least 90% identity with SEQ ID NO: 1. In some embodiments, X is a moiety comprising a sequence having at least 95% identity with SEQ ID NO: 1. In some embodiments, X is a moiety comprising a sequence having at least 96%, at least 97%, at least 98%, or at least 99% identity with SEQ ID NO: 1. In some embodiments, X is a moiety comprising the sequence of SEQ ID NO: 1. In some embodiments, X is a moiety that is the sequence of SEQ ID NO: 1.

In some embodiments of the artificial polypeptide of formula (II), Y comprises a sequence having at most 50% identity with SEQ ID NO: 2. In some embodiments, Y comprises a sequence having at most 55% identity with SEQ ID NO: 2. In some embodiments, Y comprises a sequence having at most 60% identity with SEQ ID NO: 2. In some embodiments, Y comprises a sequence having at most 65% identity with SEQ ID NO: 2. In some embodiments, Y comprises a sequence having at most 70% identity with SEQ ID NO: 2. In some embodiments, Y comprises a sequence having at most 75% identity with SEQ ID NO: 2. In some embodiments, Y comprises a sequence having at most 80% identity with SEQ ID NO: 2. In some embodiments, Y comprises a sequence having at most 85% identity with SEQ ID NO: 2. In some embodiments, Y comprises a sequence having at most 90% identity with SEQ ID NO: 2.

In some embodiments of the artificial polypeptide of formula (II), Y comprises a sequence having at least 70% identity with any one selected from SEQ ID NOs: 16-18. In some embodiments of the artificial polypeptide, Y comprises a sequence having at least 75% identity with any one selected from SEQ ID NOs: 16-18. In some embodiments of the artificial polypeptide, Y comprises a sequence having at least 80% identity with any one selected from SEQ ID NOs: 16-18. In some embodiments of the artificial polypeptide, Y comprises a sequence having at least 85% identity with any one selected from SEQ ID NOs: 16-18. In some embodiments of the artificial polypeptide, Y comprises a sequence having at least 90% identity with any one selected from SEQ ID NOs: 16-18. In some embodiments of the artificial polypeptide, Y comprises a sequence having at least 95% identity with any one selected from SEQ ID NOs: 16-18. In some embodiments of the artificial polypeptide, Y comprises a sequence having at least 96%, at least 97%, at least 98%, or at least 99% identity with any one selected from SEQ ID NOs: 16-18. In some embodiments of the artificial polypeptide, Y comprises a sequence of any one selected from SEQ ID NOs: 16-18. In some embodiments of the artificial polypeptide, Y is a sequence of any one selected from SEQ ID NOs: 16-18.

In some embodiments of the artificial polypeptide of formula (II), the artificial polypeptide comprises a sequence having at least 70% identity with any one selected from SEQ ID NOs: 42-44. In some embodiments of the artificial polypeptide, the artificial polypeptide comprises a sequence having at least 75% identity with any one selected from SEQ ID NOs: 42-44. In some embodiments of the artificial polypeptide, the artificial polypeptide comprises a sequence having at least 80% identity with any one selected from SEQ ID NOs: 42-44. In some embodiments of the artificial polypeptide, the artificial polypeptide comprises a sequence having at least 85% identity with any one selected from SEQ ID NOs: 42-44. In some embodiments of the artificial polypeptide, the artificial polypeptide comprises a sequence having at least 90% identity with any one selected from SEQ ID NOs: 42-44. In some embodiments of the artificial polypeptide, the artificial polypeptide comprises a sequence having at least 95% identity with any one selected from SEQ ID NOs: 42-44. In some embodiments of the artificial polypeptide, the artificial polypeptide comprises a sequence having at least 96%, at least 97%, at least 98%, or at least 99% identity with any one selected from SEQ ID NOs: 42-44. In some embodiments of the artificial polypeptide, the artificial polypeptide comprises a sequence of any one selected from SEQ ID NOs: 42-44. In some embodiments of the artificial polypeptide, the artificial polypeptide is a sequence of any one selected from SEQ ID NOs: 42-44.

In another aspect, provided is a method for treating or preventing allergic conjunctivitis in a subject in need thereof, comprising administering to the subject an effective amount of an artificial polypeptide of formula (III):

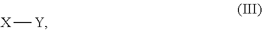

wherein X is a moiety comprising 40 to 65 amino acids, at least 50% of which are selected from H, R, K, D, Q, N and E; and
Y is a moiety comprising a sequence having at least 80% identity with SEQ ID NO: 2,
wherein a total number of H, R, K, D, Q, N and E in X is less than 33.

In some embodiments, X of formula (III) is hydrophilic moiety, at least 50% of which are selected from H, R, K, D, Q, N and E. In some embodiments, X of formula (III) can be a hydrophilic moiety, at least 55% of which are selected from R, K, N, D, Q, E, and H. In some embodiments, X of formula (III) can be a hydrophilic moiety, at least 60% of which are selected from R, K, N, D, Q, E, and H. In some embodiments, X of formula (III) can be a hydrophilic moiety, at least 65% of which are selected from R, K, N, D, Q, E, and H. In some embodiments, X of formula (III) can be a hydrophilic moiety, at least 70% of which are selected from R, K, N, D, Q, E, and H. In some embodiments, X of formula (III) can be a hydrophilic moiety, at least 75% of which are selected from R, K, N, D, Q, E, and H. In some embodiments, X of formula (III) can be a hydrophilic moiety, at least 80% of which are selected from R, K, N, D, Q, E, and H.

In some embodiments, X of formula (III) can be a hydrophilic moiety, at least 85% of which are selected from R, K, N, D, Q, E, and H. In some embodiments, X of formula (III) can be a hydrophilic moiety, at least 90% of which are selected from R, K, N, D, Q, E, and H.

In some embodiments, X of formula (III) is a moiety comprising 40 to 65 amino acids, and a total number of H, R, K, D, Q, N and E in X is less than 33. In some embodiments, X of formula (III) is a moiety comprising 40 to 65 amino acids, and a total number of H, R, K, D, Q, N and E in X is less than 32. In some embodiments, X of formula (III) is a moiety comprising 40 to 65 amino acids, and a total number of H, R, K, D, Q, N and E in X is less than 31. In some embodiments, X of formula (III) is a moiety comprising 40 to 65 amino acids, and a total number of H, R, K, D, Q, N and E in X is less than 30. In some embodiments, X of formula (III) is a moiety comprising 40 to 65 amino acids, and a total number of H, R, K, D, Q, N and E in X is less than 29. In some embodiments, X of formula (III) is a moiety comprising 40 to 65 amino acids, and a total number of H, R, K, D, Q, N and E in X is less than 28. In some embodiments, X of formula (III) is a moiety comprising 40 to 65 amino acids, and a total number of H, R, K, D, Q, N and E in X is less than 27. In some embodiments, X of formula (III) is a moiety comprising 40 to 65 amino acids, and a total number of H, R, K, D, Q, N and E in X is less than 26. In some embodiments, X of formula (III) is a moiety comprising 40 to 65 amino acids, and a total number of H, R, K, D, Q, N and E in X is less than 25. In some embodiments, X of formula (III) is a moiety comprising 40 to 65 amino acids, and a total number of H, R, K, D, Q, N and E in X is less than 24. In some embodiments, X of formula (III) is a moiety comprising 40 to 65 amino acids, and a total number of H, R, K, D, Q, N and E in X is less than 23. In some embodiments, X of formula (III) is a moiety comprising 40 to 65 amino acids, and a total number of H, R, K, D, Q, N and E in X is less than 22. In some embodiments, X of formula (III) is a moiety comprising 40 to 65 amino acids, and a total number of H, R, K, D, Q, N and E in X is less than 21. In some embodiments, X of formula (III) is a moiety comprising 40 to 65 amino acids, and a total number of H, R, K, D, Q, N and E in X is less than 20.

In some embodiments, X of formula (III) is a moiety comprising 40 to 65 amino acids, and a total number of hydrophilic amino acids in X is more than 10. In some embodiments, X of formula (III) is a moiety comprising 40 to 65 amino acids, and a total number of hydrophilic amino acids in X is more than 11. In some embodiments, X of formula (III) is a moiety comprising 40 to 65 amino acids, and a total number of hydrophilic amino acids in X is more than 12. In some embodiments, X of formula (III) is a moiety comprising 40 to 65 amino acids, and a total number of hydrophilic amino acids in X is more than 13. In some embodiments, X of formula (III) is a moiety comprising 40 to 65 amino acids, and a total number of hydrophilic amino acids in X is more than 14. In some embodiments, X of formula (III) is a moiety comprising 40 to 65 amino acids, and a total number of hydrophilic amino acids in X is more than 15. In some embodiments, X of formula (III) is a moiety comprising 40 to 65 amino acids, and a total number of hydrophilic amino acids in X is more than 16. In some embodiments, X of formula (III) is a moiety comprising 40 to 65 amino acids, and a total number of hydrophilic amino acids in X is more than 17. In some embodiments, X of formula (III) is a moiety comprising 40 to 65 amino acids, and a total number of hydrophilic amino acids in X is more than 18. In some embodiments, X of formula (III) is a moiety comprising 40 to 65 amino acids, and a total number of hydrophilic amino acids in X is more than 19. In some embodiments, X of formula (III) is a moiety comprising 40 to 65 amino acids, and a total number of hydrophilic amino acids in X is more than 20. In some embodiments, X of formula (III) is a moiety comprising 40 to 65 amino acids, and a total number of hydrophilic amino acids in X is more than 21. In some embodiments, X of formula (III) is a moiety comprising 40 to 65 amino acids, and a total number of hydrophilic amino acids in X is more than 22. In some embodiments, X of formula (III) is a moiety comprising 40 to 65 amino acids, and a total number of hydrophilic amino acids in X is more than 23. In some embodiments, X of formula (III) is a moiety comprising 40 to 65 amino acids, and a total number of hydrophilic amino acids in X is more than 24. In some embodiments, X of formula (III) is a moiety comprising 40 to 65 amino acids, and a total number of hydrophilic amino acids in X is more than 25.

In some embodiments, X of formula (III) is a moiety comprising 40 to 65 amino acids, and a total number of hydrophobic amino acids in X is no more than 15. In some embodiments, X of formula (III) is a moiety comprising 40 to 65 amino acids, and a total number of hydrophobic amino acids in X is no more than 14. In some embodiments, X of formula (III) is a moiety comprising 40 to 65 amino acids, and a total number of hydrophobic amino acids in X is no more than 13. In some embodiments, X of formula (III) is a moiety comprising 40 to 65 amino acids, and a total number of hydrophobic amino acids in X is no more than 12. In some embodiments, X of formula (III) is a moiety comprising 40 to 65 amino acids, and a total number of hydrophobic amino acids in X is no more than 11. In some embodiments, X of formula (III) is a moiety comprising 40 to 65 amino acids, and a total number of hydrophobic amino acids in X is no more than 10. In some embodiments, X of formula (III) is a moiety comprising 40 to 65 amino acids, and a total number of hydrophobic amino acids in X is no more than 5.

In some embodiments, X of formula (III) comprises a sequence having at least 70% identity with any one selected from SEQ ID NOs: 23-27. In some embodiments, X of formula (III) comprises a sequence having at least 75% identity with any one selected from SEQ ID NOs: 23-27. In some embodiments, X of formula (III) comprises a sequence having at least 80% identity with any one selected from SEQ ID NOs: 23-27. In some embodiments, X of formula (III) comprises a sequence having at least 85% identity with any one selected from SEQ ID NOs: 23-27. In some embodiments, X of formula (III) comprises a sequence having at least 90% identity with any one selected from SEQ ID NOs: 23-27. In some embodiments, X of formula (III) comprises a sequence having at least 95% identity with any one selected from SEQ ID NOs: 23-27. In some embodiments, X of formula (III) comprises a sequence having at least 96%, at least 97%, at least 98%, or at least 99% identity with any one selected from SEQ ID NOs: 23-27. In some embodiments, X of formula (III) comprises a sequence of any one selected from SEQ ID NOs: 23-27. In some embodiments, X of formula (III) is a sequence of any one selected from SEQ ID NOs: 23-27.

In some embodiments, Y of formula (III) is a moiety comprising a sequence having at least 70% identity with SEQ ID NO: 2. In some embodiments, Y of formula (III) is a moiety comprising a sequence having at least 75% identity with SEQ ID NO: 2. In some embodiments, Y of formula (III) is a moiety comprising a sequence having at least 80% identity with SEQ ID NO: 2. In some embodiments, Y of formula (III) is a moiety comprising a sequence having at least 85% identity with SEQ ID NO: 2. In some embodiments, Y of formula (III) is a moiety comprising a sequence having at least 90% identity with SEQ ID NO: 2. In some embodiments, Y of formula (III) is a moiety comprising a sequence having at least 95% identity with SEQ ID NO: 2. In some embodiments, Y of formula (III) is a moiety comprising a sequence having at least 96%, at least 97%, at least 98%, or at least 99% identity with SEQ ID NO: 2. In some embodiments, Y of formula (III) is a moiety comprising a sequence of SEQ ID NO: 2. In some embodiments, Y of formula (III) is a moiety that is a sequence of SEQ ID NO: 2.

In some embodiments, Y of formula (III) is hydrophobic moiety, at least 50% amino acids of Y are selected from I, V, L, F, C, M, and A. In some embodiments, Y of formula (III) is hydrophobic moiety, at least 55% amino acids of Y are selected from I, V, L, F, C, M, and A. In some embodiments, Y of formula (III) is hydrophobic moiety, at least 60% amino acids of Y are selected from I, V, L, F, C, M, and A. In some embodiments, Y of formula (III) is hydrophobic moiety, at least 65% amino acids of Y are selected from I, V, L, F, C, M, and A. In some embodiments, Y of formula (III) is hydrophobic moiety, at least 70% amino acids of Y are selected from I, V, L, F, C, M, and A. In some embodiments, Y of formula (III) is hydrophobic moiety, at least 75% amino acids of Y are selected from I, V, L, F, C, M, and A. In some embodiments, Y of formula (III) is hydrophobic moiety, at least 80% amino acids of Y are selected from I, V, L, F, C, M, and A. In some embodiments, Y of formula (III) is hydrophobic moiety, at least 85% amino acids of Y are selected from I, V, L, F, C, M, and A. In some embodiments, Y of formula (III) is hydrophobic moiety, at least 90% amino acids of Y are selected from I, V, L, F, C, M, and A.

In some embodiments, the artificial polypeptide of formula (III) comprises a sequence having at least 70% identity with any one selected from SEQ ID NOs: 49-53. In some embodiments, the artificial polypeptide of formula (III) comprises a sequence having at least 75% identity with any one selected from SEQ ID NOs: 49-53. In some embodiments, the artificial polypeptide of formula (III) comprises a sequence having at least 80% identity with any one selected from SEQ ID NOs: 49-53. In some embodiments, the artificial polypeptide of formula (III) comprises a sequence having at least 85% identity with any one selected from SEQ ID NOs: 49-53. In some embodiments, the artificial polypeptide of formula (III) comprises a sequence having at least 90% identity with any one selected from SEQ ID NOs: 49-53. In some embodiments, the artificial polypeptide of formula (III) comprises a sequence having at least 95% identity with any one selected from SEQ ID NOs: 49-53. In some embodiments, the artificial polypeptide of formula (III) comprises a sequence having at least 96%, at least 97%, at least 98%, or at least 99% identity with any one selected from SEQ ID NOs: 49-53. In some embodiments, the artificial polypeptide of formula (III) comprises a sequence of any one selected from SEQ ID NOs: 49-53. In some embodiments, the artificial polypeptide of formula (III) is a sequence of any one selected from SEQ ID NOs: 49-53.

In another aspect, provided is method for treating or preventing allergic conjunctivitis in a subject in need thereof, comprising administering to the subject an effective amount of an artificial polypeptide of formula (IV):

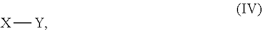

wherein X is a moiety comprising 40 to 65 amino acids, at least 50% of which are selected from H, R, K, D, Q, N and E; and
Y is a moiety comprising a sequence having at least 80% identity with SEQ ID NO: 2,
wherein X comprises a sequence having at most 90% identity with SEQ ID NO: 1.

In some embodiments, X of formula (IV) is a hydrophilic moiety, at least 50% of which are selected from H, R, K, D, Q, N and E. In some embodiments, X of formula (IV) can be a hydrophilic moiety, at least 55% of which are selected from R, K, N, D, Q, E, and H. In some embodiments, X of formula (IV) can be a hydrophilic moiety, at least 60% of which are selected from R, K, N, D, Q, E, and H. In some embodiments, X of formula (IV) can be a hydrophilic moiety, at least 65% of which are selected from R, K, N, D, Q, E, and H. In some embodiments, X of formula (IV) can be a hydrophilic moiety, at least 70% of which are selected from R, K, N, D, Q, E, and H. In some embodiments, X of formula (IV) can be a hydrophilic moiety, at least 75% of which are selected from R, K, N, D, Q, E, and H. In some embodiments, X of formula (IV) can be a hydrophilic moiety, at least 80% of which are selected from R, K, N, D, Q, E, and H. In some embodiments, X of formula (IV) can be a hydrophilic moiety, at least 85% of which are selected from R, K, N, D, Q, E, and H. In some embodiments, X of formula (IV) can be a hydrophilic moiety, at least 90% of which are selected from R, K, N, D, Q, E, and H.

In some embodiments, X of formula (IV) comprises 40 to 65 amino acids, and a total number of hydrophilic amino acids in X is more than 10. In some embodiments, X of formula (IV) comprises 40 to 65 amino acids, and a total number of hydrophilic amino acids in X is more than 15. In some embodiments, X of formula (IV) comprises 40 to 65 amino acids, and a total number of hydrophilic amino acids in X is more than 20. In some embodiments, X of formula (IV) comprises 40 to 65 amino acids, and a total number of hydrophilic amino acids in X is more than 25. In some embodiments, X of formula (IV) comprises 40 to 65 amino acids, and a total number of hydrophilic amino acids in X is more than 30. In some embodiments, X of formula (IV) comprises 40 to 65 amino acids, and a total number of hydrophilic amino acids in X is more than 35.

In some embodiments, X of formula (IV) comprises a sequence having at most 50% identity with SEQ ID NO: 1. In some embodiments, X of formula (IV) comprises a sequence having at most 55% identity with SEQ ID NO: 1. In some embodiments, X of formula (IV) comprises a sequence having at most 60% identity with SEQ ID NO: 1. In some embodiments, X of formula (IV) comprises a sequence having at most 65% identity with SEQ ID NO: 1. In some embodiments, X of formula (IV) comprises a sequence having at most 70% identity with SEQ ID NO: 1. In some embodiments, X of formula (IV) comprises a sequence having at most 75% identity with SEQ ID NO: 1. In some embodiments, X of formula (IV) comprises a sequence having at most 80% identity with SEQ ID NO: 1. In some embodiments, X of formula (IV) comprises a sequence having at most 85% identity with SEQ ID NO: 1. In some embodiments, X of formula (IV) comprises a sequence having at most 90% identity with SEQ ID NO: 1.

In some embodiments, Y of formula (IV) is a moiety comprising a sequence having at least 70% identity with SEQ ID NO: 2. In some embodiments, Y of formula (IV) is a moiety comprising a sequence having at least 75% identity with SEQ ID NO: 2. In some embodiments, Y of formula (IV) is a moiety comprising a sequence having at least 80% identity with SEQ ID NO: 2. In some embodiments, Y of formula (IV) is a moiety comprising a sequence having at least 85% identity with SEQ ID NO: 2. In some embodiments, Y of formula (IV) is a moiety comprising a sequence having at least 90% identity with SEQ ID NO: 2. In some embodiments, Y of formula (IV) is a moiety comprising a sequence having at least 95% identity with SEQ ID NO: 2. In some embodiments, Y of formula (IV) is a moiety comprising a sequence having at least 96%, at least 97%, at least 98%, or at least 99% identity with SEQ ID NO: 2. In some embodiments, Y of formula (IV) is a moiety comprising a sequence of SEQ ID NO: 2. In some embodiments, Y of formula (IV) is a moiety that is a sequence of SEQ ID NO: 2.

In some embodiments, Y of formula (IV) is hydrophobic moiety, at least 50% amino acids of Y are selected from I, V, L, F, C, M, and A. In some embodiments, Y of formula (IV) is hydrophobic moiety, at least 55% amino acids of Y are selected from I, V, L, F, C, M, and A. In some embodiments, Y of formula (IV) is hydrophobic moiety, at least 60% amino acids of Y are selected from I, V, L, F, C, M, and A. In some embodiments, Y of formula (IV) is hydrophobic moiety, at least 65% amino acids of Y are selected from I, V, L, F, C, M, and A. In some embodiments, Y of formula (IV) is hydrophobic moiety, at least 70% amino acids of Y are selected from I, V, L, F, C, M, and A. In some embodiments, Y of formula (IV) is hydrophobic moiety, at least 75% amino acids of Y are selected from I, V, L, F, C, M, and A. In some embodiments, Y of formula (IV) is hydrophobic moiety, at least 80% amino acids of Y are selected from I, V, L, F, C, M, and A. In some embodiments, Y of formula (IV) is hydrophobic moiety, at least 85% amino acids of Y are selected from I, V, L, F, C, M, and A. In some embodiments, Y of formula (IV) is hydrophobic moiety, at least 90% amino acids of Y are selected from I, V, L, F, C, M, and A.

In some embodiments, X of formula (IV) comprises a sequence having at least 70% identity with any one selected from SEQ IDs NO. 19-22 and 76-79. In some embodiments, X of formula (IV) comprises a sequence having at least 75% identity with any one selected from SEQ IDs NO. 19-22 and 76-79. In some embodiments, X of formula (IV) comprises a sequence having at least 80% identity with any one selected from SEQ IDs NO. 19-22 and 76-79. In some embodiments, X of formula (IV) comprises a sequence having at least 85% identity with any one selected from SEQ IDs NO. 19-22 and 76-79. In some embodiments, X of formula (IV) comprises a sequence having at least 90% identity with any one selected from SEQ IDs NO. 19-22 and 76-79. In some embodiments, X of formula (IV) comprises a sequence having at least 95% identity with any one selected from SEQ IDs NO. 19-22 and 76-79. In some embodiments, X of formula (IV) comprises a sequence having at least 96%, at least 97%, at least 98%, or at least 99% identity with any one selected from SEQ IDs NO. 19-22 and 76-79. In some embodiments, X of formula (IV) comprises a sequence of any one selected from SEQ IDs NO. 19-22 and 76-79. In some embodiments, X of formula (IV) is a sequence of any one selected from SEQ IDs NO. 19-22 and 76-79.

In some embodiments, the artificial polypeptide of formula (IV) comprises a sequence having at least 70% identity with any one selected from SEQ ID NOs: 45-48 and 86-89. In some embodiments, the artificial polypeptide of formula (IV) comprises a sequence having at least 75% identity with any one selected from SEQ ID NOs: 45-48 and 86-89. In some embodiments, the artificial polypeptide of formula (IV) comprises a sequence having at least 80% identity with any one selected from SEQ ID NOs: 45-48 and 86-89. In some embodiments, the artificial polypeptide of formula (IV) comprises a sequence having at least 85% identity with any one selected from SEQ ID NOs: 45-48 and 86-89. In some embodiments, the artificial polypeptide of formula (IV) comprises a sequence having at least 90% identity with any one selected from SEQ ID NOs: 45-48 and 86-89. In some embodiments, the artificial polypeptide of formula (IV) comprises a sequence having at least 95% identity with any one selected from SEQ ID NOs: 45-48 and 86-89. In some embodiments, the artificial polypeptide of formula (IV) comprises a sequence having at least 96%, at least 97%, at least 98%, or at least 99% identity with any one selected from SEQ ID NOs: 45-48 and 86-89. In some embodiments, the artificial polypeptide of formula (IV) comprises a sequence of any one selected from SEQ ID NOs: 45-48 and 86-89. In some embodiments, the artificial polypeptide of formula (IV) is a sequence of any one selected from SEQ ID NOs: 45-48 and 86-89.

In another aspect, provided is method for treating or preventing allergic conjunctivitis in a subject in need thereof, comprising administering to the subject an effective amount of an artificial polypeptide of formula (V):

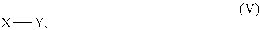

wherein X is a moiety comprising a sequence having at least 80% identity with SEQ ID NO: 1, and
Y is a moiety comprising 10 to 30 amino acids, wherein Y comprises a sequence having at least 10 continuous AAs of SEQ ID NO: 2.

In some embodiments, Y of formula (V) comprises 10 to 25 amino acids. In some embodiments, Y comprises 10 to 20 amino acids. In some embodiments, Y comprises 10 to 15 amino acids. In some embodiments, Y comprises 10, 11, 12, 13, 14, 15, 16, 17, 18, 19, 20, 21, 22, 23, 24, 25, 26, 27, 28, 29 or 30 amino acids.

In some embodiments, X of formula (V) can be a moiety comprising a sequence having at least 80% identity with SEQ ID NO: 1. In some embodiments, X is a moiety comprising a sequence having at least 85% identity with SEQ ID NO: 1. In some embodiments, X is a moiety comprising a sequence having at least 90% identity with SEQ ID NO: 1. In some embodiments, X is a moiety comprising a sequence having at least 95% identity with SEQ ID NO: 1. In some embodiments, X is a moiety comprising a sequence having at least 96%, at least 97%, at least 98%, or at least 99% identity with SEQ ID NO: 1. In some embodiments, X is a moiety comprising the sequence of SEQ ID NO: 1. In some embodiments, X is a moiety that is the sequence of SEQ ID NO: 1.

In some embodiments of the artificial polypeptide of formula (V), at least 50% amino acids of X are selected from R, K, N, D, Q, E, and H. In some embodiments, at least 55% amino acids of X are selected from R, K, N, D, Q, E, and H. In some embodiments, at least 60% amino acids of X are selected from R, K, N, D, Q, E, and H. In some embodiments, at least 65% amino acids of X are selected from R, K, N, D, Q, E, and H. In some embodiments, at least 70% amino acids of X are selected from R, K, N, D, Q, E, and H. In some embodiments, at least 75% amino acids of X are selected from R, K, N, D, Q, E, and H. In some embodiments, at least 80% amino acids of X are selected from R, K, N, D, Q, E, and H. In some embodiments, at least 85% amino acids of X are selected from R, K, N, D, Q, E, and H. In some embodiments, at least 90% amino acids of X are selected from R, K, N, D, Q, E, and H.

In some embodiments of the artificial polypeptide of formula (V), X comprises a sequence having at least 70% identity with SEQ ID NO: 96. In some embodiments of the artificial polypeptide of formula (V), X comprises a sequence having at least 75% identity with SEQ ID NO: 96. In some embodiments of the artificial polypeptide of formula (V), X comprises a sequence having at least 80% identity with SEQ ID NO: 96. In some embodiments, X comprises a sequence having at least 85% identity with SEQ ID NO: 96.

In some embodiments, X comprises a sequence having at least 90% identity with SEQ ID NO: 96. In some embodiments, X comprises a sequence having at least 95% identity with SEQ ID NO: 96. In some embodiments, X comprises a sequence having at least 96%, at least 97%, at least 98%, or at least 99% identity with SEQ ID NO: 96. In some embodiments, X comprises a sequence of SEQ ID NO: 96. In some embodiments, X is a sequence of SEQ ID NO: 96.

In some embodiments of the artificial polypeptide of formula (V), Y comprises a sequence having at most 50% identity with SEQ ID NO: 2. In some embodiments, Y comprises a sequence having at most 55% identity with SEQ ID NO: 2. In some embodiments, Y comprises a sequence having at most 60% identity with SEQ ID NO: 2. In some embodiments, Y comprises a sequence having at most 65% identity with SEQ ID NO: 2. In some embodiments, Y comprises a sequence having at most 70% identity with SEQ ID NO: 2. In some embodiments, Y comprises a sequence having at most 75% identity with SEQ ID NO: 2. In some embodiments, Y comprises a sequence having at most 80% identity with SEQ ID NO: 2. In some embodiments, Y comprises a sequence having at most 85% identity with SEQ ID NO: 2. In some embodiments, Y comprises a sequence having at most 90% identity with SEQ ID NO: 2.

In some embodiments of the artificial polypeptide of formula (V), Y comprises a sequence having at least 70% identity with any one selected from SEQ ID NOs: 97-103. In some embodiments of the artificial polypeptide of formula (V), Y comprises a sequence having at least 75% identity with any one selected from SEQ ID NOs: 97-103. In some embodiments of the artificial polypeptide of formula (V), Y comprises a sequence having at least 80% identity with any one selected from SEQ ID NOs: 97-103. In some embodiments, Y comprises a sequence having at least 85% identity with any one selected from SEQ ID NOs: 97-103. In some embodiments, Y comprises a sequence having at least 90% identity with any one selected from SEQ ID NOs: 97-103. In some embodiments, Y comprises a sequence having at least 95% identity with any one selected from SEQ ID NOs: 97-103. In some embodiments, Y comprises a sequence having at least 96%, at least 97%, at least 98%, or at least 99% identity with any one selected from SEQ ID NOs: 97-103. In some embodiments, Y comprises a sequence that is any one selected from SEQ ID NOs: 97-103. In some embodiments, Y is a sequence that is any one selected from SEQ ID NOs: 97-103.

In some embodiments, the artificial polypeptide of formula (V) comprises a sequence having at least 70% identity with any one selected from SEQ ID NOs:53-56 and 104-110. In some embodiments, the artificial polypeptide of formula (V) comprises a sequence having at least 75% identity with any one selected from SEQ ID NOs:53-56 and 104-110. In some embodiments, the artificial polypeptide of formula (V) comprises a sequence having at least 80% identity with any one selected from SEQ ID NOs:53-56 and 104-110. In some embodiments, the artificial polypeptide of formula (V) comprises a sequence having at least 85% identity with any one selected from SEQ ID NOs:53-56 and 104-110. In some embodiments, the artificial polypeptide of formula (V) comprises a sequence having at least 90% identity with any one selected from SEQ ID NOs:53-56 and 104-110. In some embodiments, the artificial polypeptide of formula (V) comprises a sequence having at least 95% identity with any one selected from SEQ ID NOs:53-56 and 104-110. In some embodiments, the artificial polypeptide of formula (V) comprises a sequence having at least 96%, at least 97%, at least 98%, or at least 99% identity with any one selected from SEQ ID NOs:53-56 and 104-110. In some embodiments, the artificial polypeptide of formula (V) comprises a sequence of any one selected from SEQ ID NOs:53-56 and 104-110. In some embodiments, the artificial polypeptide of formula (V) is a sequence of any one selected from SEQ ID NOs:53-56 and 104-110.

In another aspect, provided is a method for treating or preventing allergic conjunctivitis in a subject in need thereof, comprising administering to the subject an effective amount of an artificial polypeptide of formula (VI):

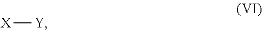

wherein X is a moiety comprising a mutant of the sequence SEQ ID NO: 1, characterized in that the mutant has at least 1, 2, 3, 4, 5, 6, 7, 8, or 9 D in SEQ ID NO: 1 mutated to S, and/or at least 1, 2, 3, 4, 5, or 6 E in SEQ ID NO: 1 mutated to T; and
Y is a moiety comprising a mutant of the sequence SEQ ID NO: 2, characterized in that the mutant has at least 1, 2, 3, 4, or 5 C in SEQ ID NO: 2 mutated to A.

In some embodiments, X of formula (VI) comprises a sequence having at least 70% identity with SEQ ID NO: 1. In some embodiments, X of formula (VI) comprises a sequence having at least 75% identity with SEQ ID NO: 1. In some embodiments, X of formula (VI) comprises a sequence having at least 80% identity with SEQ ID NO: 1. In some embodiments, X of formula (VI) comprises a sequence having at least 85% identity with SEQ ID NO: 1. In some embodiments, X of formula (VI) comprises a sequence having at least 90% identity with SEQ ID NO: 1. In some embodiments, X of formula (VI) comprises a sequence having at least 95% identity with SEQ ID NO: 1. In some embodiments, X of formula (VI) comprises a sequence having at most 95% identity with SEQ ID NO: 1. In some embodiments, X of formula (VI) comprises a sequence having at most 90% identity with SEQ ID NO: 1. In some embodiments, X of formula (VI) comprises a sequence having at most 85% identity with SEQ ID NO: 1. In some embodiments, X of formula (VI) comprises a sequence having at most 80% identity with SEQ ID NO: 1. In some embodiments, X of formula (VI) comprises a sequence having at most 75% identity with SEQ ID NO: 1.

In some embodiments, Y of formula (VI) is a moiety comprising a sequence having at least 70% identity with SEQ ID NO: 2. In some embodiments, Y of formula (VI) is a moiety comprising a sequence having at least 75% identity with SEQ ID NO: 2. In some embodiments, Y of formula (VI) is a moiety comprising a sequence having at least 80% identity with SEQ ID NO: 2. In some embodiments, Y of formula (VI) is a moiety comprising a sequence having at least 85% identity with SEQ ID NO: 2. In some embodiments, Y of formula (VI) is a moiety comprising a sequence having at least 90% identity with SEQ ID NO: 2. In some embodiments, Y of formula (VI) is a moiety comprising a sequence having at least 95% identity with SEQ ID NO: 2. In some embodiments, Y of formula (VI) comprises a sequence having at most 95% identity with SEQ ID NO: 2. In some embodiments, Y of formula (VI) comprises a sequence having at most 90% identity with SEQ ID NO: 2. In some embodiments, Y of formula (VI) comprises a sequence having at most 85% identity with SEQ ID NO: 2. In some embodiments, Y of formula (VI) comprises a sequence having at most 80% identity with SEQ ID NO: 2. In some embodiments, Y of formula (VI) comprises a sequence having at most 75% identity with SEQ ID NO: 2.

In some embodiments, Y of formula (VI) is hydrophobic moiety, at least 50% amino acids of Y are selected from I, V, L, F, C, M, and A. In some embodiments, Y of formula (VI) is hydrophobic moiety, at least 55% amino acids of Y are selected from I, V, L, F, C, M, and A. In some embodiments, Y of formula (VI) is hydrophobic moiety, at least 60% amino acids of Y are selected from I, V, L, F, C, M, and A. In some embodiments, Y of formula (VI) is hydrophobic moiety, at least 65% amino acids of Y are selected from I, V, L, F, C, M, and A. In some embodiments, Y of formula (VI) is hydrophobic moiety, at least 70% amino acids of Y are selected from I, V, L, F, C, M, and A. In some embodiments, Y of formula (VI) is hydrophobic moiety, at least 75% amino acids of Y are selected from I, V, L, F, C, M, and A. In some embodiments, Y of formula (VI) is hydrophobic moiety, at least 80% amino acids of Y are selected from I, V, L, F, C, M, and A. In some embodiments, Y of formula (VI) is hydrophobic moiety, at least 85% amino acids of Y are selected from I, V, L, F, C, M, and A. In some embodiments, Y of formula (VI) is hydrophobic moiety, at least 90% amino acids of Y are selected from I, V, L, F, C, M, and A.

In some embodiments, X of formula (VI) is a moiety comprising 40 to 65 amino acids, and a total number of R, K, T, A, N, Q, D, E, S, and G in X is more than 30. In some embodiments, X of formula (VI) is a moiety comprising 40 to 65 amino acids, and a total number of R, K, T, A, N, Q, D, E, S, and G in X is 31, 32, 33, 34, 35, 36, 37, 38, 39 or 40. In some embodiments, X of formula (VI) is a moiety comprising 40 to 65 amino acids, and a total number of W, Y, F, M, L, I, and V in X is no more than 20. In some embodiments, X of formula (VI) is a moiety comprising 40 to 65 amino acids, and a total number of W, Y, F, M, L, I, and V in X is 19, 18, 17, 16, 15 or 14.

In some embodiments, Y of formula (VI) is a moiety comprising 10 to 50 amino acids, and a total number of R, K, T, A, N, Q, D, E, S, and G in Y is more than 10. In some embodiments, Y of formula (VI) is a moiety comprising 10 to 50 amino acids, and a total number of R, K, T, A, N, Q, D, E, S, and G in Y is 11, 12, 13, 14 or 15. In some embodiments, Y of formula (VI) is a moiety comprising 10 to 50 amino acids, and a total number of W, Y, F, M, L, I, and V in X is no more than 20. In some embodiments, Y of formula (VI) is a moiety comprising 10 to 50 amino acids, and a total number of W, Y, F, M, L, I, and V in Y is 19, 18, 17, 16, 15 or 14.

In some embodiments, X of formula (VI) comprises a sequence having at least 70% identity with any one selected from SEQ ID NOs: 80-83. In some embodiments, X of formula (VI) comprises a sequence having at least 75% identity with any one selected from SEQ ID NOs: 80-83. In some embodiments, X of formula (VI) comprises a sequence having at least 80% identity with any one selected from SEQ ID NOs: 80-83. In some embodiments, X of formula (VI) comprises a sequence having at least 85% identity with any one selected from SEQ ID NOs: 80-83. In some embodiments, X of formula (VI) comprises a sequence having at least 90% identity with any one selected from SEQ ID NOs: 80-83. In some embodiments, X of formula (VI) comprises a sequence having at least 95% identity with any one selected from SEQ ID NOs: 80-83. In some embodiments, X of formula (VI) comprises a sequence having at least 96%, at least 97%, at least 98%, or at least 99% identity with any one selected from SEQ ID NOs: 80-83. In some embodiments, X of formula (VI) comprises a sequence of any one selected from SEQ ID NOs: 80-83. In some embodiments, X of formula (VI) is a sequence of any one selected from SEQ ID NOs: 80-83.

In some embodiments, Y of formula (VI) comprises a sequence having at least 70% identity with SEQ ID NO: 3. In some embodiments, Y of formula (VI) comprises a sequence having at least 75% identity with SEQ ID NO: 3. In some embodiments, Y of formula (VI) comprises a sequence having at least 80% identity with SEQ ID NO: 3. In some embodiments, Y of formula (VI) comprises a sequence having at least 85% identity with SEQ ID NO: 3. In some embodiments, Y of formula (VI) comprises a sequence having at least 90% identity with SEQ ID NO: 3. In some embodiments, Y of formula (VI) comprises a sequence having at least 95% identity with SEQ ID NO: 3. In some embodiments, Y of formula (VI) comprises a sequence having at least 96%, at least 97%, at least 98%, or at least 99% identity with SEQ ID NO: 3. In some embodiments, Y of formula (VI) comprises a sequence of SEQ ID NO: 3. In some embodiments, Y of formula (VI) is a sequence of SEQ ID NO: 3.

In some embodiments, the artificial polypeptide of formula (VI) comprises a sequence having at least 70% identity with any one selected from SEQ ID NOs: 90-93. In some embodiments, the artificial polypeptide of formula (VI) comprises a sequence having at least 75% identity with any one selected from SEQ ID NOs: 90-93. In some embodiments, the artificial polypeptide of formula (VI) comprises a sequence having at least 80% identity with any one selected from SEQ ID NOs: 90-93. In some embodiments, the artificial polypeptide of formula (VI) comprises a sequence having at least 85% identity with any one selected from SEQ ID NOs: 90-93. In some embodiments, the artificial polypeptide of formula (VI) comprises a sequence having at least 90% identity with any one selected from SEQ ID NOs: 90-93. In some embodiments, the artificial polypeptide of formula (VI) comprises a sequence having at least 95% identity with any one selected from SEQ ID NOs: 90-93. In some embodiments, the artificial polypeptide of formula (VI) comprises a sequence having at least 96%, at least 97%, at least 98%, or at least 99% identity with any one selected from SEQ ID NOs: 90-93. In some embodiments, the artificial polypeptide of formula (VI) comprises a sequence that is any one selected from SEQ ID NOs: 90-93. In some embodiments, the artificial polypeptide of formula (VI) is a sequence that is any one selected from SEQ ID NOs: 90-93.

In one aspect, provided is a method for treating or preventing allergic conjunctivitis in a subject in need thereof, comprising administering to the subject an effective amount of an artificial polypeptide of formula (VII):

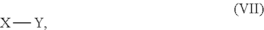

wherein X is a moiety comprising a mutant of the sequence SEQ ID NO: 1, characterized in that the mutant has at least 1, 2, 3, 4, or 5 amino acid insertions relative to SEQ ID NO: 1, and
Y is a moiety comprising 10 to 30 amino acids, wherein Y comprises a sequence having at least 15 continuous AAs of SEQ ID NO: 2.

In some embodiments, X is a moiety comprising a mutant of the sequence SEQ ID NO: 1, characterized in that the mutant has at least 1, 2, 3, 4, or 5 amino acid insertions relative to SEQ ID NO: 1.

In some embodiments, X is a moiety comprising a mutant of the sequence SEQ ID NO: 1, characterized in that the mutant has at least 1 amino acid insertion relative to SEQ ID NO: 1. In some embodiments, X is a moiety comprising a mutant of the sequence SEQ ID NO: 1, characterized in that the mutant has 1 amino acid insertion relative to SEQ ID NO: 1. In some embodiments, X is a moiety comprising a mutant of the sequence SEQ ID NO: 1, characterized in that the mutant has at least 2 amino acid insertions relative to SEQ ID NO: 1. In some embodiments, X is a moiety comprising a mutant of the sequence SEQ ID NO: 1, characterized in that the mutant has 2 amino acid insertions relative to SEQ ID NO: 1. In some embodiments, X is a moiety comprising a mutant of the sequence SEQ ID NO: 1, characterized in that the mutant has at least 3 amino acid insertions relative to SEQ ID NO: 1. In some embodiments, X is a moiety comprising a mutant of the sequence SEQ ID NO: 1, characterized in that the mutant has 3 amino acid insertions relative to SEQ ID NO: 1. In some embodiments, X is a moiety comprising a mutant of the sequence SEQ ID NO: 1, characterized in that the mutant has at least 4 amino acid insertions relative to SEQ ID NO: 1. In some embodiments, X is a moiety comprising a mutant of the sequence SEQ ID NO: 1, characterized in that the mutant has 4 amino acid insertions relative to SEQ ID NO: 1. In some embodiments, X is a moiety comprising a mutant of the sequence SEQ ID NO: 1, characterized in that the mutant has at least 5 amino acid insertions relative to SEQ ID NO: 1. In some embodiments, X is a moiety comprising a mutant of the sequence SEQ ID NO: 1, characterized in that the mutant has 5 amino acid insertions relative to SEQ ID NO: 1.

In some embodiments, at least 20% amino acids of X in the mutant are selected from R, K, N, D, Q, E, and H. In some embodiments, at least 25% amino acids of X in the mutant are selected from R, K, N, D, Q, E, and H. In some embodiments, at least 30% amino acids of X in the mutant are selected from R, K, N, D, Q, E, and H. In some embodiments, at least 35% amino acids of X in the mutant are selected from R, K, N, D, Q, E, and H. In some embodiments, at least 40% amino acids of X in the mutant are selected from R, K, N, D, Q, E, and H. In some embodiments, at least 45% amino acids of X in the mutant are selected from R, K, N, D, Q, E, and H. In some embodiments, at least 50% amino acids of X in the mutant are selected from R, K, N, D, Q, E, and H. In some embodiments, at most 55% amino acids of X in the mutant are selected from R, K, N, D, Q, E, and H. In some embodiments, at most 60% amino acids of X in the mutant are selected from R, K, N, D, Q, E, and H. In some embodiments, at most 65% amino acids of X in the mutant are selected from R, K, N, D, Q, E, and H. In some embodiments, at most 70% amino acids of X in the mutant are selected from R, K, N, D, Q, E, and H. In some embodiments, at most 75% amino acids of X in the mutant are selected from R, K, N, D, Q, E, and H. In some embodiments, at most 80% amino acids of X in the mutant are selected from R, K, N, D, Q, E, and H. In some embodiments, at most 85% amino acids of X in the mutant are selected from R, K, N, D, Q, E, and H. In some embodiments, at most 90% amino acids of X in the mutant are selected from R, K, N, D, Q, E, and H.

In some embodiments, Y is a moiety comprising 10 to 30 amino acids. In some embodiments, Y is a moiety comprising 10 amino acids, 11 amino acids, 12 amino acids, 13 amino acids, 14 amino acids, 15 amino acids, 16 amino acids, 17 amino acids, 18 amino acids, 19 amino acids, 20 amino acids, 21 amino acids, 22 amino acids, 23 amino acids, 24 amino acids, 25 amino acids, 26 amino acids, 27 amino acids, 28 amino acids, 29 amino acids, or 30 amino acids. In some embodiments, Y is a moiety comprising 11 to 29 amino acids. In some embodiments, Y is a moiety comprising 12 to 28 amino acids. In some embodiments, Y is a moiety comprising 13 to 27 amino acids. In some embodiments, Y is a moiety comprising 14 to 26 amino acids. In some embodiments, Y is a moiety comprising 15 to 25 amino acids. In some embodiments, Y comprises 15 to 25 amino acids. In some embodiments, Y comprises 18 to 25 amino acids. In some embodiments, Y comprises 20 to 25 amino acids.

In some embodiments, Y comprises a sequence having at least 15 continuous AAs of SEQ ID NO: 2. In some embodiments, Y comprises a sequence having 15 continuous AAs of SEQ ID NO: 2. In some embodiments, Y comprises a sequence having at least 16 continuous AAs of SEQ ID NO: 2. In some embodiments, Y comprises a sequence having 16 continuous AAs of SEQ ID NO: 2. In some embodiments, Y comprises a sequence having at least 17 continuous AAs of SEQ ID NO: 2. In some embodiments, Y comprises a sequence having 17 continuous AAs of SEQ ID NO: 2. In some embodiments, Y comprises a sequence having at least 18 continuous AAs of SEQ ID NO: 2. In some embodiments, Y comprises a sequence having 18 continuous AAs of SEQ ID NO: 2. In some embodiments, Y comprises a sequence having at least 19 continuous AAs of SEQ ID NO: 2. In some embodiments, Y comprises a sequence having 19 continuous AAs of SEQ ID NO: 2. In some embodiments, Y comprises a sequence having at least 20 continuous AAs of SEQ ID NO: 2. In some embodiments, Y comprises a sequence having 20 continuous AAs of SEQ ID NO: 2. In some embodiments, Y comprises a sequence having at least 21 continuous AAs of SEQ ID NO: 2. In some embodiments, Y comprises a sequence having 21 continuous AAs of SEQ ID NO: 2. In some embodiments, Y comprises a sequence having at least 22 continuous AAs of SEQ ID NO: 2. In some embodiments, Y comprises a sequence having 22 continuous AAs of SEQ ID NO: 2. In some embodiments, Y comprises a sequence having at least 23 continuous AAs of SEQ ID NO: 2. In some embodiments, Y comprises a sequence having 23 continuous AAs of SEQ ID NO: 2. In some embodiments, Y comprises a sequence having at least 24 continuous AAs of SEQ ID NO: 2. In some embodiments, Y comprises a sequence having 24 continuous AAs of SEQ ID NO: 2. In some embodiments, Y comprises a sequence having at least 25 continuous AAs of SEQ ID NO: 2. In some embodiments, Y comprises a sequence having 25 continuous AAs of SEQ ID NO: 2.

In some embodiments, Y is a moiety comprising a mutant of the sequence SEQ ID NO: 2, characterized in that the mutant has at least 1, 2, 3, 4, 5, 6, 7, 8, 9, 10, 11, 12, 13, 14, 15, 16, 17, 18, 19, or 20 amino acid truncations relative to SEQ ID NO: 2. In some embodiments, the at least 1, 2, 3, 4, 5, 6, 7, 8, 9, 10, 11, 12, 13, 14, 15, 16, 17, 18, 19, or 20 amino acid truncations are located at the C-terminal of Y. In some embodiments, Y is a moiety comprising a mutant of the sequence SEQ ID NO: 2, characterized in that the mutant has 1, 2, 3, 4, 5, 6, 7, 8, 9, 10, 11, 12, 13, 14, 15, 16, 17, 18, 19, or 20 amino acid truncations relative to SEQ ID NO: 2. In some embodiments, the 1, 2, 3, 4, 5, 6, 7, 8, 9, 10, 11, 12, 13, 14, 15, 16, 17, 18, 19, or 20 amino acid truncations are located at the C-terminal of Y.

In some embodiments, a total number of E or D in X is at least 8. In some embodiments, a total number of E or D in X is 8. In some embodiments, a total number of E or D in X is at least 9. In some embodiments, a total number of E or D in X is 9. In some embodiments, a total number of E or D in X is at least 10. In some embodiments, a total number of E or D in X is 10. In some embodiments, a total number of E or D in X is at least 11. In some embodiments, a total number of E or D in X is 11. In some embodiments, a total number of E or D in X is at least 12. In some embodiments, a total number of E or D in X is 12. In some embodiments, a total number of E or D in X is at most 15. In some embodiments, a total number of E or D in X is 15. In some embodiments, a total number of E or D in X is at most 14. In some embodiments, a total number of E or D in X is 14. In some embodiments, a total number of E or D in X is at most 13. In some embodiments, a total number of E or D in X is 13. In some embodiments, a total number of E or D in X is at most 12. In some embodiments, a total number of E or D in X is 12. In some embodiments, a total number of E or D in X is at most 11. In some embodiments, a total number of E or D in X is 11. In some embodiments, a total number of E or D in X is at most 10. In some embodiments, a total number of E or D in X is 10.

In some embodiments, X of formula (VII) is a moiety comprising 40 to 65 amino acids, and a total number of R, K, T, A, N, Q, D, E, S, and G in X is more than 30. In some embodiments, X of formula (VII) is a moiety comprising 40 to 65 amino acids, and a total number of R, K, T, A, N, Q, D, E, S, and G in X is 31, 32, 33, 34, 35, 36, 37, 38, 39 or 40. In some embodiments, X of formula (VII) is a moiety comprising 40 to 65 amino acids, and a total number of W, Y, F, M, L, I, and V in X is no more than 20. In some embodiments, X of formula (VII) is a moiety comprising 40 to 65 amino acids, and a total number of W, Y, F, M, L, I, and V in X is 19, 18, 17, 16, 15 or 14.

In some embodiments, 40-65% amino acids of Y are selected from I, V, L, F, C, M, and A. In some embodiments, at least 40% amino acids of Y are selected from I, V, L, F, C, M, and A. In some embodiments, at least 45% amino acids of Y are selected from I, V, L, F, C, M, and A. In some embodiments, at least 50% amino acids of Y are selected from I, V, L, F, C, M, and A. In some embodiments, at least 55% amino acids of Y are selected from I, V, L, F, C, M, and A. In some embodiments, at least 60% amino acids of Y are selected from I, V, L, F, C, M, and A. In some embodiments, at most 65% amino acids of Y are selected from I, V, L, F, C, M, and A. In some embodiments, at most 60% amino acids of Y are selected from I, V, L, F, C, M, and A. In some embodiments, at most 55% amino acids of Y are selected from I, V, L, F, C, M, and A. In some embodiments, at most 50% amino acids of Y are selected from I, V, L, F, C, M, and A. In some embodiments, at most 45% amino acids of Y are selected from I, V, L, F, C, M, and A.

In some embodiments, a total number of R, K, T, A, N, Q, D, E, S, and G in Y is no more than 10.

In some embodiments, a total number of R, K, T, A, N, Q, D, E, S, and G in Y is 1, 2, 3, 4, 5, 6, 7, 8, 9 or 10. In some embodiments, a total number of W, Y, F, M, L, I, and V in Y is no more than 15. In some embodiments, a total number of W, Y, F, M, L, I, and V in Y is 5, 6, 7, 8, 9, 10, 11, 12, 13, 14 or 15.

In some embodiments, X of formula (VII) comprises a sequence having at least 70% identity with SEQ ID NO: 96. In some embodiments, X of formula (VII) comprises a sequence having at least 75% identity with SEQ ID NO: 96. In some embodiments, X of formula (VII) comprises a sequence having at least 80% identity with SEQ ID NO: 96. In some embodiments, X of formula (VII) comprises a sequence having at least 85% identity with SEQ ID NO: 96. In some embodiments, X of formula (VII) comprises a sequence having at least 90% identity with SEQ ID NO: 96. In some embodiments, X of formula (VII) comprises a sequence having at least 95% identity with SEQ ID NO: 96. In some embodiments, X of formula (VII) comprises a sequence having at least 96%, at least 97%, at least 98%, or at least 99% identity with SEQ ID NO: 96. In some embodiments, X of formula (VII) comprises a sequence of SEQ ID NO: 96.

In some embodiments, X of formula (VII) is a sequence of SEQ ID NO: 96.

In some embodiments, Y of formula (VII) comprises a sequence having at least 70% identity with any one selected from SEQ ID NOs: 97-103. In some embodiments, Y of formula (VII) comprises a sequence having at least 75% identity with any one selected from SEQ ID NOs: 97-103. In some embodiments, Y of formula (VII) comprises a sequence having at least 80% identity with any one selected from SEQ ID NOs: 97-103. In some embodiments, Y of formula (VII) comprises a sequence having at least 85% identity with any one selected from SEQ ID NOs: 97-103. In some embodiments, Y of formula (VII) comprises a sequence having at least 90% identity with any one selected from SEQ ID NOs: 97-103. In some embodiments, Y of formula (VII) comprises a sequence having at least 95% identity with any one selected from SEQ ID NOs: 97-103. In some embodiments, Y of formula (VII) comprises a sequence having at least 96%, at least 97%, at least 98%, or at least 99% identity with any one selected from SEQ ID NOs: 97-103. In some embodiments, Y of formula (VII) comprises a sequence that is any one selected from SEQ ID NOs: 97-103. In some embodiments, Y of formula (VII) is a sequence that is any one selected from SEQ ID NOs: 97-103.

In some embodiments, the artificial polypeptide of formula (VII) comprises a sequence having at least 70% identity with any one selected from SEQ ID NOs: 104-110. In some embodiments, the artificial polypeptide of formula (VII) comprises a sequence having at least 75% identity with any one selected from SEQ ID NOs: 104-110. In some embodiments, the artificial polypeptide of formula (VII) comprises a sequence having at least 80% identity with any one selected from SEQ ID NOs: 104-110. In some embodiments, the artificial polypeptide of formula (VII) comprises a sequence having at least 85% identity with any one selected from SEQ ID NOs: 104-110. In some embodiments, the artificial polypeptide of formula (VII) comprises a sequence having at least 90% identity with any one selected from SEQ ID NOs: 104-110. In some embodiments, the artificial polypeptide of formula (VII) comprises a sequence having at least 95% identity with any one selected from SEQ ID NOs: 104-110. In some embodiments, the artificial polypeptide of formula (VII) comprises a sequence having at least 96%, at least 97%, at least 98%, or at least 99% identity with any one selected from SEQ ID NOs: 104-110. In some embodiments, the artificial polypeptide of formula (VII) comprises a sequence that is any one selected from SEQ ID NOs: 104-110. In some embodiments, the artificial polypeptide of formula (VII) is a sequence that is any one selected from SEQ ID NOs: 104-110.

In another aspect, provided is a method for treating or preventing allergic conjunctivitis in a subject in need thereof, comprising administering to the subject an effective amount of a mutant of artificial polypeptide of formula (I),

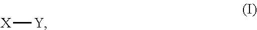

wherein X is a moiety comprising a sequence having at least 80% identity with SEQ ID NO: 1,
Y is a moiety comprising 10 to 50 amino acids, at least 50% of which are selected from I, V, L, F, C, M, and A,
Y comprises a total number of Cysteine (C) of less than 5,
characterized in that the mutant has at least 1, 2, 3, 4, 5, 6, 7, 8, or 9 D in X mutated to S, and/or at least 1, 2, 3, 4, 5, or 6 E in X mutated to T.

In some embodiments, at least 20% amino acids of X in the mutant are selected from R, K, N, D, Q, E, and H. In some embodiments, at least 25% amino acids of X in the mutant are selected from R, K, N, D, Q, E, and H. In some embodiments, at least 30% amino acids of X in the mutant are selected from R, K, N, D, Q, E, and H. In some embodiments, at least 35% amino acids of X in the mutant are selected from R, K, N, D, Q, E, and H. In some embodiments, at least 40% amino acids of X in the mutant are selected from R, K, N, D, Q, E, and H. In some embodiments, at least 45% amino acids of X in the mutant are selected from R, K, N, D, Q, E, and H. In some embodiments, at least 50% amino acids of X in the mutant are selected from R, K, N, D, Q, E, and H. In some embodiments, at most 55% amino acids of X in the mutant are selected from R, K, N, D, Q, E, and H. In some embodiments, at most 60% amino acids of X in the mutant are selected from R, K, N, D, Q, E, and H. In some embodiments, at most 65% amino acids of X in the mutant are selected from R, K, N, D, Q, E, and H. In some embodiments, at most 70% amino acids of X in the mutant are selected from R, K, N, D, Q, E, and H. In some embodiments, at most 75% amino acids of X in the mutant are selected from R, K, N, D, Q, E, and H. In some embodiments, at most 80% amino acids of X in the mutant are selected from R, K, N, D, Q, E, and H. In some embodiments, at most 85% amino acids of X in the mutant are selected from R, K, N, D, Q, E, and H. In some embodiments, at most 90% amino acids of X in the mutant are selected from R, K, N, D, Q, E, and H.

In some embodiments, X in the mutant comprises a sequence having at least 70% identity with SEQ ID NO: 1. In some embodiments, X in the mutant comprises a sequence having at least 75% identity with SEQ ID NO: 1. In some embodiments, X in the mutant comprises a sequence having at least 80% identity with SEQ ID NO: 1. In some embodiments, X in the mutant comprises a sequence having at least 85% identity with SEQ ID NO: 1. In some embodiments, X in the mutant comprises a sequence having at least 90% identity with SEQ ID NO: 1. In some embodiments, X in the mutant comprises a sequence having at least 95% identity with SEQ ID NO: 1.

In some embodiments, Y in the mutant can be a moiety comprising 10 to 50 amino acids, and a total number of Cysteine (C) of Y is 5. In some embodiments, Y in the mutant can be a moiety comprising 10 to 50 amino acids, and a total number of Cysteine (C) of Y is less than 5. In some embodiments, Y in the mutant can be a moiety comprising 10 to 50 amino acids, and a total number of Cysteine (C) of Y is 4.

In some embodiments, Y in the mutant can be a moiety comprising 10 to 50 amino acids, and a total number of Cysteine (C) of Y is less than 4. In some embodiments, Y in the mutant can be a moiety comprising 10 to 50 amino acids, and a total number of Cysteine (C) of Y is 3. In some embodiments, Y in the mutant can be a moiety comprising 10 to 50 amino acids, and a total number of Cysteine (C) of Y is less than 3. In some embodiments, Y in the mutant can be a moiety comprising 10 to 50 amino acids, and a total number of Cysteine (C) of Y is 2. In some embodiments, Y in the mutant can be a moiety comprising 10 to 50 amino acids, and a total number of Cysteine (C) of Y is less than 2. In some embodiments, Y in the mutant can be a moiety comprising 10 to 50 amino acids, and a total number of Cysteine (C) of Y is 1. In some embodiments, Y in the mutant can be a moiety comprising 10 to 50 amino acids, and a total number of Cysteine (C) of Y is less than 1. In some embodiments, Y in the mutant can be a moiety comprising 10 to 50 amino acids, and a total number of Cysteine (C) of Y is 0.

In some embodiments, a total number of hydrophobic amino acids in Y in the mutant is more than 8. In some embodiments, a total number of hydrophobic amino acids in Y in the mutant is more than 9. In some embodiments, a total number of hydrophobic amino acids in Y in the mutant is more than 10. In some embodiments, a total number of hydrophobic amino acids in Y in the mutant is more than 11. In some embodiments, the total number of hydrophobic amino acids in Y in the mutant is more than 12. In some embodiments, the total number of hydrophobic amino acids in Y in the mutant is more than 13. In some embodiments, the total number of hydrophobic amino acids in Y in the mutant is more than 14. In some embodiments, the total number of hydrophobic amino acids in Y in the mutant is more than 15.

In some embodiments, a total number of hydrophilic amino acids in Y in the mutant is 5. In some embodiments, a total number of hydrophilic amino acids in Y in the mutant is no more than 5. In some embodiments, a total number of hydrophilic amino acids in Y in the mutant is 4. In some embodiments, a total number of hydrophilic amino acids in Y in the mutant is no more than 4. In some embodiments, a total number of hydrophilic amino acids in Y in the mutant is 3. In some embodiments, a total number of hydrophilic amino acids in Y in the mutant is no more than 3. In some embodiments, a total number of hydrophilic amino acids in Y in the mutant is 2. In some embodiments, a total number of hydrophilic amino acids in Y in the mutant is no more than 2. In some embodiments, a total number of hydrophilic amino acids in Y in the mutant is 1. In some embodiments, a total number of hydrophilic amino acids in Y in the mutant is no more than 1.

In some embodiments, Y in the mutant comprises a sequence having at least 70% identity with any one selected from SEQ ID NOs: 3-15, 58-61 and 84-85. In some embodiments, Y in the mutant comprises a sequence having at least 75% identity with any one selected from SEQ ID NOs: 3-15, 58-61 and 84-85.

In some embodiments, Y in the mutant comprises a sequence having at least 80% identity with any one selected from SEQ ID NOs: 3-15, 58-61 and 84-85. In some embodiments, Y in the mutant comprises a sequence having at least 85% identity with any one selected from SEQ ID NOs: 3-15, 58-61 and 84-85. In some embodiments, Y in the mutant comprises a sequence having at least 90% identity with any one selected from SEQ ID NOs: 3-15, 58-61 and 84-85. In some embodiments, Y in the mutant comprises a sequence having at least 95% identity with any one selected from SEQ ID NOs: 3-15, 58-61 and 84-85. In some embodiments, Y in the mutant comprises a sequence having at least 96%, at least 97%, at least 98%, or at least 99% identity with any one selected from SEQ ID NOs: 3-15, 58-61 and 84-85. In some embodiments, Y in the mutant comprises a sequence of any one selected from SEQ ID NOs: 3-15, 58-61 and 84-85. In some embodiments, Y in the mutant is a sequence of any one selected from SEQ ID NOs: 3-15, 58-61 and 84-85.

In some embodiments, Y in the mutant comprises a sequence having at least 70% identity with any one selected from SEQ ID NOs: 3-18, 58-61 and 84-85. In some embodiments, Y in the mutant comprises a sequence having at least 75% identity with any one selected from SEQ ID NOs: 3-18, 58-61 and 84-85.

In some embodiments, Y in the mutant comprises a sequence having at least 80% identity with any one selected from SEQ ID NOs: 3-18, 58-61 and 84-85. In some embodiments, Y in the mutant comprises a sequence having at least 85% identity with any one selected from SEQ ID NOs: 3-18, 58-61 and 84-85. In some embodiments, Y in the mutant comprises a sequence having at least 90% identity with any one selected from SEQ ID NOs: 3-18, 58-61 and 84-85. In some embodiments, Y in the mutant comprises a sequence having at least 95% identity with any one selected from SEQ ID NOs: 3-18, 58-61 and 84-85. In some embodiments, Y in the mutant comprises a sequence having at least 96%, at least 97%, at least 98%, or at least 99% identity with any one selected from SEQ ID NOs: 3-18, 58-61 and 84-85. In some embodiments, Y in the mutant comprises a sequence of any one selected from SEQ ID NOs: 3-18, 58-61 and 84-85. In some embodiments, Y in the mutant is a sequence of any one selected from SEQ ID NOs: 3-18, 58-61 and 84-85.

In another aspect, provided is a method for treating or preventing allergic conjunctivitis in a subject in need thereof, comprising administering to the subject an effective amount of a mutant of artificial polypeptide of formula (II),

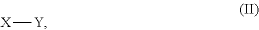

wherein X is a moiety comprising a sequence having at least 80% identity with SEQ ID NO: 1,
Y is a moiety comprising 10 to 50 amino acids, at least 50% of which are selected from I, V, L, F, C, M, and A,
Y comprises a sequence having at most 90% identity with SEQ ID NO: 2,
characterized in that the mutant has at least 1, 2, 3, 4, 5, 6, 7, 8, or 9 D in X mutated to S, and/or at least 1, 2, 3, 4, 5, or 6 E in X mutated to T.

In some embodiments, at least 20% amino acids of X in the mutant are selected from R, K, N, D, Q, E, and H. In some embodiments, at least 25% amino acids of X in the mutant are selected from R, K, N, D, Q, E, and H. In some embodiments, at least 30% amino acids of X in the mutant are selected from R, K, N, D, Q, E, and H. In some embodiments, at least 35% amino acids of X in the mutant are selected from R, K, N, D, Q, E, and H. In some embodiments, at least 40% amino acids of X in the mutant are selected from R, K, N, D, Q, E, and H. In some embodiments, at least 45% amino acids of X in the mutant are selected from R, K, N, D, Q, E, and H. In some embodiments, at least 50% amino acids of X in the mutant are selected from R, K, N, D, Q, E, and H. In some embodiments, at most 55% amino acids of X in the mutant are selected from R, K, N, D, Q, E, and H. In some embodiments, at most 60% amino acids of X in the mutant are selected from R, K, N, D, Q, E, and H. In some embodiments, at most 65% amino acids of X in the mutant are selected from R, K, N, D, Q, E, and H. In some embodiments, at most 70% amino acids of X in the mutant are selected from R, K, N, D, Q, E, and H. In some embodiments, at most 75% amino acids of X in the mutant are selected from R, K, N, D, Q, E, and H. In some embodiments, at most 80% amino acids of X in the mutant are selected from R, K, N, D, Q, E, and H. In some embodiments, at most 85% amino acids of X in the mutant are selected from R, K, N, D, Q, E, and H. In some embodiments, at most 90% amino acids of X in the mutant are selected from R, K, N, D, Q, E, and H.

In some embodiments, X in the mutant comprises a sequence having at least 70% identity with SEQ ID NO: 1. In some embodiments, X in the mutant comprises a sequence having at least 75% identity with SEQ ID NO: 1. In some embodiments, X in the mutant comprises a sequence having at least 80% identity with SEQ ID NO: 1. In some embodiments, X in the mutant comprises a sequence having at least 85% identity with SEQ ID NO: 1. In some embodiments, X in the mutant comprises a sequence having at least 90% identity with SEQ ID NO: 1. In some embodiments, X in the mutant comprises a sequence having at least 95% identity with SEQ ID NO: 1.

In some embodiments, Y in the mutant comprises a sequence having at most 50% identity with SEQ ID NO: 2. In some embodiments, Y in the mutant comprises a sequence having at most 55% identity with SEQ ID NO: 2. In some embodiments, Y in the mutant comprises a sequence having at most 60% identity with SEQ ID NO: 2. In some embodiments, Y in the mutant comprises a sequence having at most 65% identity with SEQ ID NO: 2. In some embodiments, Y in the mutant comprises a sequence having at most 70% identity with SEQ ID NO: 2. In some embodiments, Y in the mutant comprises a sequence having at most 75% identity with SEQ ID NO: 2. In some embodiments, Y in the mutant comprises a sequence having at most 80% identity with SEQ ID NO: 2. In some embodiments, Y in the mutant comprises a sequence having at most 85% identity with SEQ ID NO: 2. In some embodiments, Y in the mutant comprises a sequence having at most 90% identity with SEQ ID NO: 2.

In some embodiments, a total number of hydrophobic amino acids in Y in the mutant is more than 8. In some embodiments, a total number of hydrophobic amino acids in Y in the mutant is more than 9. In some embodiments, a total number of hydrophobic amino acids in Y in the mutant is more than 10. In some embodiments, a total number of hydrophobic amino acids in Y in the mutant is more than 11. In some embodiments, the total number of hydrophobic amino acids in Y in the mutant is more than 12. In some embodiments, the total number of hydrophobic amino acids in Y in the mutant is more than 13. In some embodiments, the total number of hydrophobic amino acids in Y in the mutant is more than 14. In some embodiments, the total number of hydrophobic amino acids in Y in the mutant is more than 15.

In some embodiments, a total number of hydrophilic amino acids in Y in the mutant is 5. In some embodiments, a total number of hydrophilic amino acids in Y in the mutant is no more than 5. In some embodiments, a total number of hydrophilic amino acids in Y in the mutant is 4. In some embodiments, a total number of hydrophilic amino acids in Y in the mutant is no more than 4. In some embodiments, a total number of hydrophilic amino acids in Y in the mutant is 3. In some embodiments, a total number of hydrophilic amino acids in Y in the mutant is no more than 3. In some embodiments, a total number of hydrophilic amino acids in Y in the mutant is 2. In some embodiments, a total number of hydrophilic amino acids in Y in the mutant is no more than 2. In some embodiments, a total number of hydrophilic amino acids in Y in the mutant is 1. In some embodiments, a total number of hydrophilic amino acids in Y in the mutant is no more than 1.

In some embodiments, Y in the mutant comprises a sequence having at least 70% identity with any one selected from SEQ ID NOs: 16-18. In some embodiments, Y in the mutant comprises a sequence having at least 75% identity with any one selected from SEQ ID NOs: 16-18. In some embodiments, Y in the mutant comprises a sequence having at least 80% identity with any one selected from SEQ ID NOs: 16-18. In some embodiments, Y in the mutant comprises a sequence having at least 85% identity with any one selected from SEQ ID NOs: 16-18. In some embodiments, Y in the mutant comprises a sequence having at least 90% identity with any one selected from SEQ ID NOs: 16-18. In some embodiments, Y in the mutant comprises a sequence having at least 95% identity with any one selected from SEQ ID NOs: 16-18. In some embodiments, Y in the mutant comprises a sequence having at least 96%, at least 97%, at least 98%, or at least 99% identity with any one selected from SEQ ID NOs: 16-18. In some embodiments, Y in the mutant comprises a sequence of any one selected from SEQ ID NOs: 16-18. In some embodiments, Y in the mutant is a sequence of any one selected from SEQ ID NOs: 16-18.

In another aspect, provided is a method for treating or preventing allergic conjunctivitis in a subject in need thereof, comprising administering to the subject an effective amount of a mutant of artificial polypeptide of formula (III),

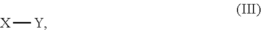

wherein X is a moiety comprising 40 to 65 amino acids, at least 50% of which are selected from H, R, K, D, Q, N and E,
Y is a moiety comprising a sequence having at least 80% identity with SEQ ID NO: 2,
a total number of H, R, K, D, Q, N and E in X is less than 33,
characterized in that the mutant has at least 1, 2, 3, 4, 5, 6, 7, 8, or 9 D in X mutated to S, and/or at least 1, 2, 3, 4, 5, or 6 E in X mutated to T.

In some embodiments, at least 20% amino acids of X in the mutant are selected from R, K, N, D, Q, E, and H. In some embodiments, at least 25% amino acids of X in the mutant are selected from R, K, N, D, Q, E, and H. In some embodiments, at least 30% amino acids of X in the mutant are selected from R, K, N, D, Q, E, and H. In some embodiments, at least 35% amino acids of X in the mutant are selected from R, K, N, D, Q, E, and H. In some embodiments, at least 40% amino acids of X in the mutant are selected from R, K, N, D, Q, E, and H. In some embodiments, at least 45% amino acids of X in the mutant are selected from R, K, N, D, Q, E, and H. In some embodiments, at least 50% amino acids of X in the mutant are selected from R, K, N, D, Q, E, and H. In some embodiments, at most 55% amino acids of X in the mutant are selected from R, K, N, D, Q, E, and H. In some embodiments, at most 60% amino acids of X in the mutant are selected from R, K, N, D, Q, E, and H. In some embodiments, at most 65% amino acids of X in the mutant are selected from R, K, N, D, Q, E, and H. In some embodiments, at most 70% amino acids of X in the mutant are selected from R, K, N, D, Q, E, and H. In some embodiments, at most 75% amino acids of X in the mutant are selected from R, K, N, D, Q, E, and H. In some embodiments, at most 80% amino acids of X in the mutant are selected from R, K, N, D, Q, E, and H. In some embodiments, at most 85% amino acids of X in the mutant are selected from R, K, N, D, Q, E, and H. In some embodiments, at most 90% amino acids of X in the mutant are selected from R, K, N, D, Q, E, and H.

In some embodiments, X in the mutant is a moiety comprising 40 to 65 amino acids, and a total number of H, R, K, D, Q, N and E in X is less than 33. In some embodiments, X in the mutant is a moiety comprising 40 to 65 amino acids, and a total number of H, R, K, D, Q, N and E in X is less than 32. In some embodiments, X in the mutant is a moiety comprising 40 to 65 amino acids, and a total number of H, R, K, D, Q, N and E in X is less than 31. In some embodiments, X in the mutant is a moiety comprising 40 to 65 amino acids, and a total number of H, R, K, D, Q, N and E in X is less than 30. In some embodiments, X in the mutant is a moiety comprising 40 to 65 amino acids, and a total number of H, R, K, D, Q, N and E in X is less than 29. In some embodiments, X in the mutant is a moiety comprising 40 to 65 amino acids, and a total number of H, R, K, D, Q, N and E in X is less than 28. In some embodiments, X in the mutant is a moiety comprising 40 to 65 amino acids, and a total number of H, R, K, D, Q, N and E in X is less than 27. In some embodiments, X in the mutant is a moiety comprising 40 to 65 amino acids, and a total number of H, R, K, D, Q, N and E in X is less than 26. In some embodiments, X in the mutant is a moiety comprising 40 to 65 amino acids, and a total number of H, R, K, D, Q, N and E in X is less than 25. In some embodiments, X in the mutant is a moiety comprising 40 to 65 amino acids, and a total number of H, R, K, D, Q, N and E in X is less than 24. In some embodiments, X in the mutant is a moiety comprising 40 to 65 amino acids, and a total number of H, R, K, D, Q, N and E in X is less than 23. In some embodiments, X in the mutant is a moiety comprising 40 to 65 amino acids, and a total number of H, R, K, D, Q, N and E in X is less than 22. In some embodiments, X in the mutant is a moiety comprising 40 to 65 amino acids, and a total number of H, R, K, D, Q, N and E in X is less than 21. In some embodiments, X in the mutant is a moiety comprising 40 to 65 amino acids, and a total number of H, R, K, D, Q, N and E in X is less than 20.

In some embodiments, X in the mutant is a moiety comprising 40 to 65 amino acids, and a total number of hydrophilic amino acids in X is more than 10. In some embodiments, X in the mutant is a moiety comprising 40 to 65 amino acids, and a total number of hydrophilic amino acids in X is more than 11. In some embodiments, X in the mutant is a moiety comprising 40 to 65 amino acids, and a total number of hydrophilic amino acids in X is more than 12. In some embodiments, X in the mutant is a moiety comprising 40 to 65 amino acids, and a total number of hydrophilic amino acids in X is more than 13. In some embodiments, X in the mutant is a moiety comprising 40 to 65 amino acids, and a total number of hydrophilic amino acids in X is more than 14. In some embodiments, X in the mutant is a moiety comprising 40 to 65 amino acids, and a total number of hydrophilic amino acids in X is more than 15. In some embodiments, X in the mutant is a moiety comprising 40 to 65 amino acids, and a total number of hydrophilic amino acids in X is more than 16. In some embodiments, X in the mutant is a moiety comprising 40 to 65 amino acids, and a total number of hydrophilic amino acids in X is more than 17. In some embodiments, X in the mutant is a moiety comprising 40 to 65 amino acids, and a total number of hydrophilic amino acids in X is more than 18. In some embodiments, X in the mutant is a moiety comprising 40 to 65 amino acids, and a total number of hydrophilic amino acids in X is more than 19. In some embodiments, X in the mutant is a moiety comprising 40 to 65 amino acids, and a total number of hydrophilic amino acids in X is more than 20. In some embodiments, X in the mutant is a moiety comprising 40 to 65 amino acids, and a total number of hydrophilic amino acids in X is more than 21. In some embodiments, X in the mutant is a moiety comprising 40 to 65 amino acids, and a total number of hydrophilic amino acids in X is more than 22. In some embodiments, X in the mutant is a moiety comprising 40 to 65 amino acids, and a total number of hydrophilic amino acids in X is more than 23. In some embodiments, X in the mutant is a moiety comprising 40 to 65 amino acids, and a total number of hydrophilic amino acids in X is more than 24. In some embodiments, X in the mutant is a moiety comprising 40 to 65 amino acids, and a total number of hydrophilic amino acids in X is more than 25.

In some embodiments, X in the mutant is a moiety comprising 40 to 65 amino acids, and a total number of hydrophobic amino acids in X is no more than 15. In some embodiments, X in the mutant is a moiety comprising 40 to 65 amino acids, and a total number of hydrophobic amino acids in X is no more than 14. In some embodiments, X in the mutant is a moiety comprising 40 to 65 amino acids, and a total number of hydrophobic amino acids in X is no more than 13. In some embodiments, X in the mutant is a moiety comprising 40 to 65 amino acids, and a total number of hydrophobic amino acids in X is no more than 12. In some embodiments, X in the mutant is a moiety comprising 40 to 65 amino acids, and a total number of hydrophobic amino acids in X is no more than 11. In some embodiments, X in the mutant is a moiety comprising 40 to 65 amino acids, and a total number of hydrophobic amino acids in X is no more than 10. In some embodiments, X in the mutant is a moiety comprising 40 to 65 amino acids, and a total number of hydrophobic amino acids in X is no more than 5.

In some embodiments, X in the mutant comprises a sequence having at least 70% identity with SEQ ID NO: 1. In some embodiments, X in the mutant comprises a sequence having at least 75% identity with SEQ ID NO: 1. In some embodiments, X in the mutant comprises a sequence having at least 80% identity with SEQ ID NO: 1. In some embodiments, X in the mutant comprises a sequence having at least 85% identity with SEQ ID NO: 1. In some embodiments, X in the mutant comprises a sequence having at least 90% identity with SEQ ID NO: 1. In some embodiments, X in the mutant comprises a sequence having at least 95% identity with SEQ ID NO: 1.

In some embodiments, Y in the mutant is a moiety comprising a sequence having at least 70% identity with SEQ ID NO: 2. In some embodiments, Y in the mutant is a moiety comprising a sequence having at least 75% identity with SEQ ID NO: 2. In some embodiments, Y in the mutant is a moiety comprising a sequence having at least 80% identity with SEQ ID NO: 2. In some embodiments, Y in the mutant is a moiety comprising a sequence having at least 85% identity with SEQ ID NO: 2. In some embodiments, Y in the mutant is a moiety comprising a sequence having at least 90% identity with SEQ ID NO: 2. In some embodiments, Y in the mutant is a moiety comprising a sequence having at least 95% identity with SEQ ID NO: 2. In some embodiments, Y in the mutant is a moiety comprising a sequence having at least 96%, at least 97%, at least 98%, or at least 99% identity with SEQ ID NO: 2. In some embodiments, Y in the mutant is a moiety comprising a sequence of SEQ ID NO: 2. In some embodiments, Y in the mutant is a moiety that is a sequence of SEQ ID NO: 2.

In some embodiments, Y in the mutant is hydrophobic moiety, at least 50% amino acids of Y are selected from I, V, L, F, C, M, and A. In some embodiments, Y in the mutant is hydrophobic moiety, at least 55% amino acids of Y are selected from I, V, L, F, C, M, and A. In some embodiments, Y in the mutant is hydrophobic moiety, at least 60% amino acids of Y are selected from I, V, L, F, C, M, and A. In some embodiments, Y in the mutant is hydrophobic moiety, at least 65% amino acids of Y are selected from I, V, L, F, C, M, and A. In some embodiments, Y in the mutant is hydrophobic moiety, at least 70% amino acids of Y are selected from I, V, L, F, C, M, and A. In some embodiments, Y in the mutant is hydrophobic moiety, at least 75% amino acids of Y are selected from I, V, L, F, C, M, and A. In some embodiments, Y in the mutant is hydrophobic moiety, at least 80% amino acids of Y are selected from I, V, L, F, C, M, and A. In some embodiments, Y in the mutant is hydrophobic moiety, at least 85% amino acids of Y are selected from I, V, L, F, C, M, and A. In some embodiments, Y in the mutant is hydrophobic moiety, at least 90% amino acids of Y are selected from I, V, L, F, C, M, and A.

In another aspect, provided is a method for treating or preventing allergic conjunctivitis in a subject in need thereof, comprising administering to the subject an effective amount of a mutant of artificial polypeptide of formula (IV),

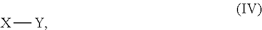

wherein X is a moiety comprising 40 to 65 amino acids, at least 50% of which are selected from H, R, K, D, Q, N and E,
Y is a moiety comprising a sequence having at least 80% identity with SEQ ID NO: 2,
X comprises a sequence having at most 90% identity with SEQ ID NO: 1,
   characterized in that the mutant has at least 1, 2, 3, 4, 5, 6, 7, 8, or 9 D in X mutated to S, and/or at least 1, 2, 3, 4, 5, or 6 E in X mutated to T.

In some embodiments, at least 20% amino acids of X in the mutant are selected from R, K, N, D, Q, E, and H. In some embodiments, at least 25% amino acids of X in the mutant are selected from R, K, N, D, Q, E, and H. In some embodiments, at least 30% amino acids of X in the mutant are selected from R, K, N, D, Q, E, and H. In some embodiments, at least 35% amino acids of X in the mutant are selected from R, K, N, D, Q, E, and H. In some embodiments, at least 40% amino acids of X in the mutant are selected from R, K, N, D, Q, E, and H. In some embodiments, at least 45% amino acids of X in the mutant are selected from R, K, N, D, Q, E, and H. In some embodiments, at least 50% amino acids of X in the mutant are selected from R, K, N, D, Q, E, and H. In some embodiments, at most 55% amino acids of X in the mutant are selected from R, K, N, D, Q, E, and H. In some embodiments, at most 60% amino acids of X in the mutant are selected from R, K, N, D, Q, E, and H. In some embodiments, at most 65% amino acids of X in the mutant are selected from R, K, N, D, Q, E, and H. In some embodiments, at most 70% amino acids of X in the mutant are selected from R, K, N, D, Q, E, and H. In some embodiments, at most 75% amino acids of X in the mutant are selected from R, K, N, D, Q, E, and H. In some embodiments, at most 80% amino acids of X in the mutant are selected from R, K, N, D, Q, E, and H. In some embodiments, at most 85% amino acids of X in the mutant are selected from R, K, N, D, Q, E, and H. In some embodiments, at most 90% amino acids of X in the mutant are selected from R, K, N, D, Q, E, and H.

In some embodiments, X in the mutant comprises 40 to 65 amino acids, and a total number of hydrophilic amino acids in X is more than 10. In some embodiments, X in the mutant comprises 40 to 65 amino acids, and a total number of hydrophilic amino acids in X is more than 15. In some embodiments, X in the mutant comprises 40 to 65 amino acids, and a total number of hydrophilic amino acids in X is more than 20. In some embodiments, X in the mutant comprises 40 to 65 amino acids, and a total number of hydrophilic amino acids in X is more than 25. In some embodiments, X in the mutant comprises 40 to 65 amino acids, and a total number of hydrophilic amino acids in X is more than 30. In some embodiments, X in the mutant comprises 40 to 65 amino acids, and a total number of hydrophilic amino acids in X is more than 35.

In some embodiments, X in the mutant is a moiety comprising 40 to 65 amino acids, and a total number of hydrophobic amino acids in X is no more than 15. In some embodiments, X in the mutant is a moiety comprising 40 to 65 amino acids, and a total number of hydrophobic amino acids in X is no more than 14. In some embodiments, X in the mutant is a moiety comprising 40 to 65 amino acids, and a total number of hydrophobic amino acids in X is no more than 13. In some embodiments, X in the mutant is a moiety comprising 40 to 65 amino acids, and a total number of hydrophobic amino acids in X is no more than 12. In some embodiments, X in the mutant is a moiety comprising 40 to 65 amino acids, and a total number of hydrophobic amino acids in X is no more than 11. In some embodiments, X in the mutant is a moiety comprising 40 to 65 amino acids, and a total number of hydrophobic amino acids in X is no more than 10. In some embodiments, X in the mutant is a moiety comprising 40 to 65 amino acids, and a total number of hydrophobic amino acids in X is no more than 5.

In some embodiments, X in the mutant comprises a sequence having at least 70% identity with SEQ ID NO: 1. In some embodiments, X in the mutant comprises a sequence having at least 75% identity with SEQ ID NO: 1. In some embodiments, X in the mutant comprises a sequence having at least 80% identity with SEQ ID NO: 1. In some embodiments, X in the mutant comprises a sequence having at least 85% identity with SEQ ID NO: 1. In some embodiments, X in the mutant comprises a sequence having at least 90% identity with SEQ ID NO: 1. In some embodiments, X in the mutant comprises a sequence having at least 95% identity with SEQ ID NO: 1.

In some embodiments, Y in the mutant is a moiety comprising a sequence having at least 70% identity with SEQ ID NO: 2. In some embodiments, Y in the mutant is a moiety comprising a sequence having at least 75% identity with SEQ ID NO: 2. In some embodiments, Y in the mutant is a moiety comprising a sequence having at least 80% identity with SEQ ID NO: 2. In some embodiments, Y in the mutant is a moiety comprising a sequence having at least 85% identity with SEQ ID NO: 2. In some embodiments, Y in the mutant is a moiety comprising a sequence having at least 90% identity with SEQ ID NO: 2. In some embodiments, Y in the mutant is a moiety comprising a sequence having at least 95% identity with SEQ ID NO: 2. In some embodiments, Y in the mutant is a moiety comprising a sequence having at least 96%, at least 97%, at least 98%, or at least 99% identity with SEQ ID NO: 2. In some embodiments, Y in the mutant is a moiety comprising a sequence of SEQ ID NO: 2. In some embodiments, Y in the mutant is a moiety that is a sequence of SEQ ID NO: 2.

In some embodiments, Y in the mutant is hydrophobic moiety, at least 50% amino acids of Y are selected from I, V, L, F, C, M, and A. In some embodiments, Y in the mutant is hydrophobic moiety, at least 55% amino acids of Y are selected from I, V, L, F, C, M, and A. In some embodiments, Y in the mutant is hydrophobic moiety, at least 60% amino acids of Y are selected from I, V, L, F, C, M, and A. In some embodiments, Y in the mutant is hydrophobic moiety, at least 65% amino acids of Y are selected from I, V, L, F, C, M, and A. In some embodiments, Y in the mutant is hydrophobic moiety, at least 70% amino acids of Y are selected from I, V, L, F, C, M, and A. In some embodiments, Y in the mutant is hydrophobic moiety, at least 75% amino acids of Y are selected from I, V, L, F, C, M, and A. In some embodiments, Y in the mutant is hydrophobic moiety, at least 80% amino acids of Y are selected from I, V, L, F, C, M, and A. In some embodiments, Y in the mutant is hydrophobic moiety, at least 85% amino acids of Y are selected from I, V, L, F, C, M, and A. In some embodiments, Y in the mutant is hydrophobic moiety, at least 90% amino acids of Y are selected from I, V, L, F, C, M, and A.

In another aspect, provided is a method for treating or preventing allergic conjunctivitis in a subject in need thereof, comprising administering to the subject an effective amount of a mutant of artificial polypeptide of formula (V),

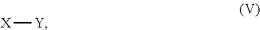

wherein X is a moiety comprising a sequence having at least 80% identity with SEQ ID NO: 1,
Y is a moiety comprising 10 to 30 amino acids, wherein Y comprises a sequence having at least 10 continuous AAs of SEQ ID NO: 2,
characterized in that the mutant has at least 1, 2, 3, 4, 5, 6, 7, 8, or 9 D in X mutated to S, and/or at least 1, 2, 3, 4, 5, or 6 E in X mutated to T.

In some embodiments, at least 20% amino acids of X in the mutant are selected from R, K, N, D, Q, E, and H. In some embodiments, at least 25% amino acids of X in the mutant are selected from R, K, N, D, Q, E, and H. In some embodiments, at least 30% amino acids of X in the mutant are selected from R, K, N, D, Q, E, and H. In some embodiments, at least 35% amino acids of X in the mutant are selected from R, K, N, D, Q, E, and H. In some embodiments, at least 40% amino acids of X in the mutant are selected from R, K, N, D, Q, E, and H. In some embodiments, at least 45% amino acids of X in the mutant are selected from R, K, N, D, Q, E, and H. In some embodiments, at least 50% amino acids of X in the mutant are selected from R, K, N, D, Q, E, and H. In some embodiments, at most 55% amino acids of X in the mutant are selected from R, K, N, D, Q, E, and H. In some embodiments, at most 60% amino acids of X in the mutant are selected from R, K, N, D, Q, E, and H. In some embodiments, at most 65% amino acids of X in the mutant are selected from R, K, N, D, Q, E, and H. In some embodiments, at most 70% amino acids of X in the mutant are selected from R, K, N, D, Q, E, and H. In some embodiments, at most 75% amino acids of X in the mutant are selected from R, K, N, D, Q, E, and H. In some embodiments, at most 80% amino acids of X in the mutant are selected from R, K, N, D, Q, E, and H. In some embodiments, at most 85% amino acids of X in the mutant are selected from R, K, N, D, Q, E, and H. In some embodiments, at most 90% amino acids of X in the mutant are selected from R, K, N, D, Q, E, and H.

In some embodiments, Y in the mutant comprises 10 to 25 amino acids. In some embodiments, Y in the mutant comprises 10 to 20 amino acids. In some embodiments, Y in the mutant comprises 10 to 15 amino acids. In some embodiments, Y in the mutant comprises 10, 11, 12, 13, 14, 15, 16, 17, 18, 19, 20, 21, 22, 23, 24, 25, 26, 27, 28, 29 or 30 amino acids.

In some embodiments, X in the mutant comprises a sequence having at least 70% identity with SEQ ID NO: 1. In some embodiments, X in the mutant comprises a sequence having at least 75% identity with SEQ ID NO: 1. In some embodiments, X in the mutant comprises a sequence having at least 80% identity with SEQ ID NO: 1. In some embodiments, X in the mutant comprises a sequence having at least 85% identity with SEQ ID NO: 1. In some embodiments, X in the mutant comprises a sequence having at least 90% identity with SEQ ID NO: 1. In some embodiments, X in the mutant comprises a sequence having at least 95% identity with SEQ ID NO: 1.

In some embodiments, Y in the mutant comprises a sequence having at most 50% identity with SEQ ID NO: 2. In some embodiments, Y in the mutant comprises a sequence having at most 55% identity with SEQ ID NO: 2. In some embodiments, Y in the mutant comprises a sequence having at most 60% identity with SEQ ID NO: 2. In some embodiments, Y in the mutant comprises a sequence having at most 65% identity with SEQ ID NO: 2. In some embodiments, Y in the mutant comprises a sequence having at most 70% identity with SEQ ID NO: 2. In some embodiments, Y in the mutant comprises a sequence having at most 75% identity with SEQ ID NO: 2. In some embodiments, Y in the mutant comprises a sequence having at most 80% identity with SEQ ID NO: 2. In some embodiments, Y in the mutant comprises a sequence having at most 85% identity with SEQ ID NO: 2. In some embodiments, Y in the mutant comprises a sequence having at most 90% identity with SEQ ID NO: 2.

In another aspect, provided is method for treating or preventing allergic conjunctivitis in a subject in need thereof, comprising administering to the subject an effective amount of a polypeptide having at least 70% identity with any one selected from SEQ ID NO: 28, SEQ ID NOs: 57 and 62-74 or a fragment or variant thereof.

In some embodiments, the polypeptide has at least 70% identity with any one selected from SEQ ID NO: 28, SEQ ID NOs: 57 and 62-74, or a fragment or variant thereof. In some embodiments, the polypeptide has at least 75% identity with any one selected from SEQ ID NO: 28, SEQ ID NOs: 57 and 62-74, or a fragment or variant thereof. In some embodiments, the polypeptide has at least 80% identity with any one selected from SEQ ID NO: 28, SEQ ID NOs: 57 and 62-74, or a fragment or variant thereof. In some embodiments, the polypeptide has at least 85% identity with any one selected from SEQ ID NO: 28, SEQ ID NOs: 57 and 62-74, or a fragment or variant thereof. In some embodiments, the polypeptide has at least 90% identity with any one selected from SEQ ID NO: 28, SEQ ID NOs: 57 and 62-74, or a fragment or variant thereof. In some embodiments, the polypeptide has at least 95% identity with any one selected from SEQ ID NO: 28, SEQ ID NOs: 57 and 62-74, or a fragment or variant thereof. In some embodiments, the polypeptide has at least 96%, at least 97%, at least 98%, or at least 99% identity with any one selected from SEQ ID NO: 28, SEQ ID NOs: 57 and 62-74, or a fragment or variant thereof. In some embodiments, the polypeptide comprises or is an amino acid sequence of any one selected from SEQ ID NO: 28, SEQ ID NOs: 57 and 62-74, or a fragment or variant thereof. In some embodiments, the polypeptide is a polypeptide of any one of SEQ ID NO: 28, SEQ ID NOs: 57 and 62-74. In some embodiments, the polypeptide is a polypeptide of any one of SEQ ID NOs: 28 and 62-74. In some embodiments, the polypeptide is a polypeptide of SEQ ID NO: 28.

In another aspect, provided is an artificial polypeptide of formula (I), (II), (III), (IV), (V), (VI), or (VII), or a mutant of artificial polypeptide of (I), (II), (III), (IV) or (V), or a polypeptide having at least 70% identity with any one selected from SEQ ID NO. 28, SEQ ID NOs. 57 and 62-74 or a fragment or variant thereof, for use in treating or preventing allergic conjunctivitis in a subject in need thereof.

In another aspect, provided is use of an artificial polypeptide of formula (I), (II), (III), (IV), (V), (VI), or (VII), or a mutant of artificial polypeptide of (I), (II), (III), (IV) or (V), or a polypeptide having at least 70% identity with any one selected from SEQ ID NO. 28, SEQ ID NOs. 57 and 62-74 or a fragment or variant thereof for the preparation of a medicament for treating or preventing allergic conjunctivitis in a subject in need thereof.

In some embodiments, the medicament is a medicament for use in human.

In some embodiments, the medicament is a veterinary medicament.

In some embodiments of either of the above aspects, the subject is a mammal. In some embodiments, the subject is any one selected from the group consisting of a human, primate, rodent, canine, feline, equine, ovine and porcine. In some embodiments, the subject is a human. In some embodiments, the subject is an infant, a toddler, a child, an adolescent, an adult, or an elderly. In some embodiments, the subject is a man or a woman. In some embodiments, the subject is a pet. In some embodiments, the subject is any one selected from the group consisting of mice, rat, guinea pig, gerbil, hamster, chinchilla, rabbit, ferret, cat, dog and pig.

In some embodiments of either of the above aspects, the allergic conjunctivitis is at least one selected from the group consisting of seasonal allergic conjunctivitis, perennial allergic conjunctivitis, vernal keratoconjunctivitis, giant papillary conjunctivitis, atopic keratoconjunctivitis and phlyctenular keratoconjunctivitis.

In some embodiments, the allergic conjunctivitis is seasonal allergic conjunctivitis. In some embodiments, the seasonal allergic conjunctivitis is caused or partially caused by a seasonal allergen. In some embodiments, the seasonal allergic conjunctivitis is caused by a seasonal allergen. In some embodiments, the seasonal allergic conjunctivitis is partially caused by a seasonal allergen. In some embodiments, the seasonal allergen is at least one selected from the group consisting of tree pollens, weed pollens, grass pollens and mold spores. In some embodiments, the seasonal allergen is selected from the group consisting of tree pollens. In some embodiments, the seasonal allergen is selected from the group consisting of weed pollens. In some embodiments, the seasonal allergen is selected from the group consisting of grass pollens. In some embodiments, the seasonal allergen is selected from the group consisting of mold spores. In some embodiments, the seasonal allergen is 2, 3 or 4 selected from the group consisting of tree pollens, weed pollens, grass pollens and mold spores.

In some embodiments, the allergic conjunctivitis is perennial allergic conjunctivitis. In some embodiments, the perennial allergic conjunctivitis is caused or partially caused by a perennial allergen. In some embodiments, the perennial allergic conjunctivitis is caused by a perennial allergen. In some embodiments, the perennial allergic conjunctivitis is partially caused by a perennial allergen. In some embodiments, the perennial allergen is at least one selected from the group consisting of dust mites, cockroaches and animal danders. In some embodiments, the perennial allergen is selected from the group consisting of dust mites. In some embodiments, the perennial allergen is selected from the group consisting of cockroaches. In some embodiments, the perennial allergen is selected from the group consisting of animal danders. In some embodiments, the animal is a companion animal, such as a cat, a dog, a rabbit, a pig, a ferret, a hamster, a gerbil, a chinchilla, a rat, a mouse, a guinea pig and a bird such as a parrot. In some embodiments, the perennial allergen is 2 or 3 selected from the group consisting of dust mites, cockroaches and animal danders.

In some embodiments, the allergic conjunctivitis is vernal keratoconjunctivitis. In some embodiments, the vernal keratoconjunctivitis is at least one selected from the group consisting of palpebral vernal keratoconjunctivitis, limbal vernal keratoconjunctivitis and mixed vernal keratoconjunctivitis. In some embodiments, the vernal keratoconjunctivitis is palpebral vernal keratoconjunctivitis. In some embodiments, the vernal keratoconjunctivitis is limbal vernal keratoconjunctivitis. In some embodiments, the vernal keratoconjunctivitis is mixed vernal keratoconjunctivitis.

In some embodiments, the allergic conjunctivitis is giant papillary conjunctivitis. In some embodiments, the giant papillary conjunctivitis is caused or partially caused by repeated mechanical irritation of the conjunctiva. In some embodiments, the giant papillary conjunctivitis is caused by repeated mechanical irritation of the conjunctiva. In some embodiments, the giant papillary conjunctivitis is partially caused by repeated mechanical irritation of the conjunctiva. In some embodiments, the repeated mechanical irritation of the conjunctiva is associated with at least one selected from the group consisting of contact lenses wearing, use of ocular prostheses, use of conjunctival sutures, use of extruding scleral buckles and elevated corneal scars. In some embodiments, the repeated mechanical irritation of the conjunctiva is associated with contact lenses wearing. In some embodiments, the contact lenses are least one selected from the group consisting of soft lenses, rigid lenses, hydrogel lenses and silicone hydrogel lenses. In some embodiments, the repeated mechanical irritation of the conjunctiva is associated with use of ocular prostheses. In some embodiments, the repeated mechanical irritation of the conjunctiva is associated with use of conjunctival sutures. In some embodiments, the repeated mechanical irritation of the conjunctiva is associated with extruding scleral buckles. In some embodiments, the repeated mechanical irritation of the conjunctiva is associated with elevated corneal scars. In some embodiments, the repeated mechanical irritation of the conjunctiva is associated with 2, 3, 4 or 5 selected from the group consisting of contact lenses wearing, use of ocular prostheses, use of conjunctival sutures, use of extruding scleral buckles and elevated corneal scars.

In some embodiments, the allergic conjunctivitis is atopic keratoconjunctivitis. In some embodiments, the atopic keratoconjunctivitis is companied with atopic dermatitis and/or asthma. In some embodiments, the atopic keratoconjunctivitis is companied with atopic dermatitis. In some embodiments, the atopic keratoconjunctivitis is companied with asthma. In some embodiments, the atopic keratoconjunctivitis is companied with atopic dermatitis and asthma.

In some embodiments, the allergic conjunctivitis is phlyctenular keratoconjunctivitis. In some embodiments, the phlyctenular keratoconjunctivitis is caused or partially caused by a delayed hypersensitivity or inflammatory reaction of the cornea and conjunctiva to antigens expressed by various pathogens. In some embodiments, the phlyctenular keratoconjunctivitis is caused by a delayed hypersensitivity or inflammatory reaction of the cornea and conjunctiva to antigens expressed by various pathogens. In some embodiments, the phlyctenular keratoconjunctivitis is partially caused by a delayed hypersensitivity or inflammatory reaction of the cornea and conjunctiva to antigens expressed by various pathogens. In some embodiments, the pathogens are at least one selected from the group consisting of bacteria (e.g., Staphylococcus aureus, Streptococcus viridians, Mycobacterium tuberculosis), viruses (e.g., Herpes simplex virus), chlamydiae, fungi (e.g., Candida such as Candida albicans) and parasites (e.g., tapeworms such as Hymenolepis nana; and nematodes). In some embodiments, the pathogens are bacteria.

In some embodiments, the bacteria are Staphylococcus aureus. In some embodiments, the bacteria are Streptococcus viridians. In some embodiments, the bacteria are Mycobacterium tuberculosis. In some embodiments, the bacteria are at least one selected from the group consisting of Staphylococcus aureus, Streptococcus viridians and Mycobacterium tuberculosis. In some embodiments, the pathogens are viruses.

In some embodiments, the viruses are Herpes simplex virus. In some embodiments, the pathogens are chlamydiae. In some embodiments, the pathogens are fungi. In some embodiments, the fungi are Candida.

In some embodiments, the Candida are Candida albicans. In some embodiments, the pathogens are parasites. In some embodiments, the parasites are intestinal parasites. In some embodiments, the parasites are tapeworms. In some embodiments, the tapeworms are Hymenolepis nana. In some embodiments, the parasites are nematodes. In some embodiments, the pathogens are 2, 3, 4 and 5 selected from the group consisting of bacteria (e.g., Staphylococcus aureus, Streptococcus viridians, Mycobacterium tuberculosis), viruses (e.g., Herpes simplex virus), chlamydiae, fungi (e.g., Candida such as Candida albicans) and parasites (e.g., tapeworms such as Hymenolepis nana; and nematodes).

In some embodiments, the subject does not have any ocular disease selected from the group consisting of viral conjunctivitis, bacterial conjunctivitis, chemical conjunctivitis and dry eye or dry eye associated disorders. In some embodiments, the subject does not have viral conjunctivitis. In some embodiments, the subject does not have bacterial conjunctivitis. In some embodiments, the subject does not have chemical conjunctivitis. In some embodiments, the subject does not have dry eye (DE) or dry eye (DE) associated disorders. In some embodiments, the dry eye (DE) or dry eye (DE) associated disorders are at least one selected from the group consisting of dry eye syndrome, dysfunctional tear syndrome, keratoconjunctivitis sicca (KCS), lacrimal keratoconjunctivitis, aqueous tear-deficient DE, evaporative DE, Sjogren's syndrome DE, non-Sjogren's syndrome DE, conjunctivitis-associated DE, post-viral conjunctivitis DE, post-cataract surgery DE, VDT operation-associated DE, contact lens wearing-associated DE, environmental DE, corneal neovascularization DE, allergic DE, LASIK-induced neurotrophic epitheliopathy, hypolacrimation, xerophthalmia, Stevens-Johnson syndrome, ocular cicatricial pemphigoid, ocular pemphigoid blepharitis marginal, eyelid-closure failure, corneal ulcer, blepharitis and eye redness.

In some embodiments, the subject experiences at least one symptom selected from the group consisting of redness of conjunctiva, edema of conjunctiva, hyperemia of conjunctiva, redness of eyelids, edema of eyelids, hyperemia of eyelids, ocular itching, visible damage of cornea, mucous discharge, watery discharge, tearing, photophobia, foreign body sensation and difficulty in opening eyes before administrating of an artificial polypeptide of formula (I), (II), (III), (IV), (V), (VI), or (VII), or a mutant of artificial polypeptide of (I), (II), (III), (IV) or (V), or a polypeptide having at least 70% identity with any one selected from SEQ ID NO. 28, SEQ ID NOs. 57 and 62-74 or a fragment or variant thereof (“an artificial polypeptide of formula (I), (II), (III), (IV), (V), (VI), or (VII), or a mutant of artificial polypeptide of (I), (II), (III), (IV) or (V), or a polypeptide having at least 70% identity with any one selected from SEQ ID NO. 28, SEQ ID NOs. 57 and 62-74 or a fragment or variant thereof” is collectively called “the polypeptides of the present disclosure” for short hereinafter). In some embodiments, the subject experiences redness of conjunctiva before administrating of the polypeptides of the present disclosure. In some embodiments, the subject experiences edema of conjunctiva before administrating of the polypeptides of the present disclosure. In some embodiments, the subject experiences hyperemia of conjunctiva before administrating of the polypeptides of the present disclosure. In some embodiments, the subject experiences redness of eyelids before administrating of the polypeptides of the present disclosure. In some embodiments, the subject experiences edema of eyelids before administrating of the polypeptides of the present disclosure. In some embodiments, the subject experiences hyperemia of eyelids before administrating of the polypeptides of the present disclosure. In some embodiments, the subject experiences ocular itching before administrating of the polypeptides of the present disclosure. In some embodiments, the subject experiences visible damage of cornea before administrating of the polypeptides of the present disclosure. In some embodiments, the subject experiences mucous discharge before administrating of the polypeptides of the present disclosure.

In some embodiments, the subject experiences watery discharge before administrating of the polypeptides of the present disclosure. In some embodiments, the subject experiences tearing before administrating of the polypeptides of the present disclosure. In some embodiments, the subject experiences photophobia before administrating of the polypeptides of the present disclosure. In some embodiments, the subject experiences foreign body sensation before administrating of the polypeptides of the present disclosure. In some embodiments, the subject experiences difficulty in opening eyes before administrating of the polypeptides of the present disclosure. In some embodiments, the subject experiences 2, 3, 4, 5, 6, 7, 8, 9, 10, 11, 12, 13 or 14 symptoms selected from the group consisting of redness of conjunctiva, edema of conjunctiva, hyperemia of conjunctiva, redness of eyelids, edema of eyelids, hyperemia of eyelids, ocular itching, visible damage of cornea, mucous discharge, watery discharge, tearing, photophobia, foreign body sensation and difficulty in opening eyes before administrating of the polypeptides of the present disclosure.

In some embodiments, the subject displays reduction in at least one symptom selected from the group consisting of redness of conjunctiva, edema of conjunctiva, hyperemia of conjunctiva, redness of eyelids, edema of eyelids, hyperemia of eyelids, ocular itching, visible damage of cornea, mucous discharge, watery discharge, tearing, photophobia, foreign body sensation and difficulty in opening eyes after administrating of at least one dose of the polypeptides of the present disclosure. In some embodiments, the subject displays reduction in redness of conjunctiva after administrating of at least one dose of the polypeptides of the present disclosure. In some embodiments, the subject displays reduction in edema of conjunctiva after administrating of at least one dose of the polypeptides of the present disclosure. In some embodiments, the subject displays reduction in hyperemia of conjunctiva after administrating of at least one dose of the polypeptides of the present disclosure. In some embodiments, the subject displays reduction in redness of eyelids after administrating of at least one dose of the polypeptides of the present disclosure.

In some embodiments, the subject displays reduction in edema of eyelids after administrating of at least one dose of the polypeptides of the present disclosure. In some embodiments, the subject displays reduction in hyperemia of eyelids after administrating of at least one dose of the polypeptides of the present disclosure.

In some embodiments, the subject displays reduction in ocular itching after administrating of at least one dose of the polypeptides of the present disclosure. In some embodiments, the subject displays reduction in visible damage of cornea after administrating of at least one dose of the polypeptides of the present disclosure. In some embodiments, the subject displays reduction in mucous discharge after administrating of at least one dose of the polypeptides of the present disclosure. In some embodiments, the subject displays reduction in watery discharge after administrating of at least one dose of the polypeptides of the present disclosure. In some embodiments, the subject displays reduction in tearing after administrating of at least one dose of the polypeptides of the present disclosure. In some embodiments, the subject displays reduction in photophobia after administrating of at least one dose of the polypeptides of the present disclosure. In some embodiments, the subject displays reduction in foreign body sensation after administrating of at least one dose of the polypeptides of the present disclosure. In some embodiments, the subject displays reduction in difficulty in opening eyes after administrating of at least one dose of the polypeptides of the present disclosure. In some embodiments, the subject displays reduction in 2, 3, 4, 5, 6, 7, 8, 9, 10, 11, 12, 13 or 14 symptoms selected from the group consisting of redness of conjunctiva, edema of conjunctiva, hyperemia of conjunctiva, redness of eyelids, edema of eyelids, hyperemia of eyelids, ocular itching, visible damage of cornea, mucous discharge, watery discharge, tearing, photophobia, foreign body sensation and difficulty in opening eyes after administrating of at least one dose of the polypeptides of the present disclosure.

In some embodiments, the subject displays improvements in at least one tissue selected from the group consisting of conjunctiva, cornea, anterior chamber, iris, pupil, vitreum vitreous and fundus after administrating of at least one dose of the polypeptides of the present disclosure. In some embodiments, the subject displays improvements in conjunctiva after administrating of at least one dose of the polypeptides of the present disclosure. In some embodiments, the subject displays improvements in cornea after administrating of at least one dose of the polypeptides of the present disclosure. In some embodiments, the subject displays improvements in anterior chamber after administrating of at least one dose of the polypeptides of the present disclosure. In some embodiments, the subject displays improvements in iris after administrating of at least one dose of the polypeptides of the present disclosure. In some embodiments, the subject displays improvements in pupil after administrating of at least one dose of the polypeptides of the present disclosure. In some embodiments, the subject displays improvements in vitreum vitreous after administrating of at least one dose of the polypeptides of the present disclosure. In some embodiments, the subject displays improvements in fundus after administrating of at least one dose of the polypeptides of the present disclosure. In some embodiments, the subject displays improvements in 2, 3, 4, 5, 6 or 7 tissues selected from the group consisting of conjunctiva, cornea, anterior chamber, iris, pupil, vitreum vitreous and fundus after administrating of at least one dose of the polypeptides of the present disclosure.

In some embodiments, the subject displays improved inflammation scores after administrating of at least one dose of the polypeptides of the present disclosure.

In some embodiments, the subject displays improvement in at least one pathological tissue selected from the group consisting of conjunctiva, cornea, anterior chamber, iris, pupil, vitreum vitreous and fundus after administrating of at least one dose of the polypeptides of the present disclosure.

In some embodiments, the subject displays a certain degree of recovery from corneal injury after administrating of at least one dose of the polypeptides of the present disclosure.

In some embodiments, the subject does not experience death attributable to administration of at least one dose of the polypeptides of the present disclosure.

In some embodiments, the subject does not experience poor mental state attributable to administration of at least one dose of the polypeptides of the present disclosure.

In some embodiments, the subject does not experience altered (e.g., deteriorated) behavioral activity attributable to administration of at least one dose of the polypeptides of the present disclosure.

In some embodiments, the subject does not experience altered (e.g., deteriorated) eating situation attributable to administration of at least one dose of the polypeptides of the present disclosure.

In some embodiments, the subject does not experience altered (e.g., deteriorated) fecal property attributable to administration of at least one dose of the polypeptides of the present disclosure.

In some embodiments, the subject does not experience weight loss attributable to administration of at least one dose of the polypeptides of the present disclosure.

In some embodiments, the polypeptides of the present disclosure are administered in the form of an ophthalmic formulation comprising the polypeptides of the present disclosure and a pharmaceutically acceptable excipient.

In some embodiments, the ophthalmic formulation is formulated as a solution. In some embodiments, the ophthalmic formulation is formulated as an eye drop solution. In some embodiments, the ophthalmic formulation is formulated as a gel. In some embodiments, the ophthalmic formulation is formulated as an ointment. In some embodiments, the ophthalmic formulation is formulated as a suspension, a semi-liquid, a semi-solid gel, a cream, a foam gel, a contact lens solution, an eyewash, and the like.

In some embodiments, the ophthalmic formulation is prepared by dissolving the polypeptides of the present disclosure in an aqueous solution. Aqueous solutions and diluents that can be used in preparing the ophthalmic formulation include but are not limited to distilled water, physiological saline, and the like.

In some embodiments, the ophthalmic formulation is prepared by dissolving the polypeptides of the present disclosure in a non-aqueous solution or diluents. Non-aqueous solutions and diluents include but are not limited to edible (e.g. vegetable) oil, liquid paraffin, mineral oil, propylene glycol, p-octyldodecanol, polysorbate, macrogols, aluminum monostearate and the like.

In some embodiments, the ophthalmic formulation is formulated by admixing, diluting or dissolving the polypeptides of the present disclosure, with appropriate pharmaceutically acceptable excipients, such as disintegrators, binders, lubricants, diluents, buffers, antiseptics, moistening agents, emulsifiers, dispersing agents, stabilizing agents and dissolving aids in accordance with conventional methods, and in a conventional manner depending upon the dosage form.

In some embodiments, buffering agents are added to keep the pH constant and can include pharmaceutically acceptable buffering agents such as borate buffer, citrate buffer, tartrate buffer, phosphate buffer, acetate buffer and a Tris-HCl buffer (comprising tris(hydroxymethyl) aminomethane and HCl). For example, a Tris-HCl buffer having pH of 7.4 comprises 3 g/l of tris-(hydroxymethyl)-aminomethane and 0.76 g/l of HCl. In some embodiments, the buffer is 10× phosphate buffer saline (“PBS”) or 5×PBS solution. Buffering agents are added to the ophthalmic formulation in an amount that provides sufficient buffer capacity for the expected physiological conditions.

Other buffers that can be used in the ophthalmic formulation include, but are not limited to, buffers based on HEPES (N-{2-hydroxyethyl}piperazine-N′-{2-ethanesulfonic acid}) having pKa of 7.5 at 25° C. and pH in the range of about 6.8-8.2; BES (N,N-bis{2-hydroxyethyl}2-aminoethanesulfonic acid) having pKa of 7.1 at 25° C. and pH in the range of about 6.4-7.8; MOPS (3-{N-morpholino}propanesulfonic acid) having pKa of 7.2 at 25° C. and pH in the range of about 6.5-7.9; TES (N-tris{hydroxymethyl}-methyl-2-aminoethanesulfonic acid) having pKa of 7.4 at 25° C. and pH in the range of about 6.8-8.2; MOBS (4-{N-morpholino}butanesulfonic acid) having pKa of 7.6 at 25° C. and pH in the range of about 6.9-8.3; DIPSO (3-(N,N-bis{2-hydroxyethyl}amino)-2-hydroxypropane)) having pKa of 7.52 at 25° C. and pH in the range of about 7-8.2; TAPS ({(2-hydroxy-3 {tris(hydroxymethyl)methylamino}-1-propanesulfonic acid)) having pKa of 7.61 at 25° C. and pH in the range of about 7-8.2; TAPS ({(2-hydroxy-1,1-bis(hydroxymethyl)ethyl)aminol-l-propanesulfonic acid)) having pKa of 8.4 at 25° C. and pH in the range of about 7.7-9.1; TABS (N-tris(hydroxymethyl)methyl-4-aminobutanesulfonic acid) having pKa of 8.9 at 25° C. and pH in the range of about 8.2-9.6; AMPSO (N-(1,1-dimethyl-2-hydroxyethyl)-3-amino-2-hydroxypropanesulfonic acid)) having pKa of 9.0 at 25° C. and pH in the range of about 8.3-9.7; CHES (2-cyclohexylamino)ethanesulfonic acid) having pKa of 9.5 at 25° C. and pH in the range of about 8.6-10.0; CAPSO (3-(cyclohexylamino)-2-hydroxy-1-propanesulfonic acid) having pKa of 9.6 at 25° C. and pH in the range of about 8.9-10.3; and CAPS (3-(cyclohexylamino)-1-propane sulfonic acid) having pKa of 10.4 at 25° C. and pH in the range of about 9.7-11.1.

In some embodiments, isotonizers can be added to make the ophthalmic formulation isotonic with the tear. Isotonizers include, but are not limited to, sugars such as dextrose, glucose, sucrose and fructose; sugar alcohols such as mannitol and sorbitol; polyhydric alcohols such as glycerol, polyethylene glycol and propylene glycol; and salts such as sodium chloride, sodium citrate, benzalkonium chloride, phedrine chloride, potassium chloride, procaine chloride, chloram phenicol, and sodium succinate. Isotonizers are added in an amount that makes the osmotic pressure of the ophthalmic formulation equal to that of the tear.

In some embodiments, the ophthalmic formulation comprises a tonicity agent. Tonicity agents suitable for the ophthalmic formulation include but are not limited to sodium chloride, sodium nitrate, sodium sulfate, sodium bisulfate, potassium chloride, calcium chloride, magnesium chloride, zinc chloride, potassium acetate, sodium acetate, sodium bicarbonate, sodium carbonate, sodium thiosulfate, magnesium sulfate, disodium hydrogen phosphate, sodium dihydrogen phosphate, potassium dihydrogen phosphate, dextrose, mannitol, sorbitol, dextrose, sucrose, urea, propylene glycol, glycerin, and a combination thereof.

In some embodiments, the ophthalmic formulation comprises a viscosity-enhancing agent.

Viscosity-enhancing agents suitable for the ophthalmic formulation include but are not limited to monomeric polyols such as tyloxapol, glycerol, propylene glycol, ethylene glycol; polymeric polyols such as polyethylene glycol (e.g., PEG 300, PEG 400); cellulose-based polymer such as cellulose gum, alkylcellulose, hydroxyl-alkyl cellulose, hydroxyl-alkyl alkylcellulose, carboxy-alkyl cellulose, hydroxyethylcellulose hypromellose, hydroxypropylmethyl cellulose, methylcellulose, carboxymethylcellulose sodium, hydroxylpropylcellulose; dextrans such as dextran 70; water-soluble proteins such as gelatin; vinyl polymers such as polyvinyl alcohol, polyvinyl pyrollidine; other polyols such as polysorbate 80, povidone; polysaccharides and glycosaminoglycans such as hyaluronan, chondroitin sulfate; and combinations thereof.

In some embodiments, preservatives can be added to maintain the integrity of the ophthalmic formulation. Preservatives include, but are not limited to, sorbic acid, benzalkonium chloride, benzododecinium bromide, parabens, chlorobutanol, benzylic alcohol, phenylethyl alcohol, edentate disodium, sorbic acid, polyquaternium-1, or other agents known to those skilled in the art.

In addition to the above, in some embodiments, it is desirable to use additional agents which include, but are not limited to, stabilizers suitable for the ophthalmic formulation such as sodium sulfite, sodium carbonate, and propylene glycol; antioxidants such as ascorbic acid, sodium ascorbate, butylated hydroxy toluene (BHT), butylated hydroxyanisole (BHA), tocopherol, sodium thiosulfate; and/or chelating agents such as ethylene-diamine-tetra-acetic acid (EDTA), ethylene glycol-bis-(2-aminoethyl)-N,N,N,N-tetraacetic acid (EGTA) and sodium citrate.

Eye drops, ophthalmic gels and/or ophthalmic ointments can be prepared by aseptic manipulation. Alternatively, sterilization of the composition can be performed at a suitable stage of preparation. In some embodiments, the sterile composition can be prepared by mixing sterile ingredients aseptically. In some embodiments, the sterile composition can be prepared by first mixing the ingredients then sterilizing the final preparation. Sterilization methods can include, but are not limited to, heat sterilization, irradiation and filtration.

In some embodiments, ophthalmic ointments (eye ointments) can be aseptically prepared by mixing the polypeptides of the present disclosure into a base that is used for preparation of eye ointments followed by formulation into pharmaceutical preparations with any method known in the art. Typical bases for eye ointments are exemplified by vaseline, jelene 50, plastibase and macrogol. In addition, surfactants may be added to increase hydrophilia.

In some embodiments, additives may be added to the ophthalmic formulation such as eye drops, ophthalmic gels and/or ophthalmic ointments as needed. In some embodiments, the additives include but are not limited to additional ingredients, additives, carrier suitable for use in contact on or around the eye without undue toxicity, incompatibility, instability, irritation, allergic response, and the like.

In some embodiments, the ophthalmic formulation is formulated for topical administration. In some embodiments, the ophthalmic formulation can be locally administered to the eye, such as subconjunctivally, retrobulbarly, periocularly, subretinally, suprachoroidally, or intraocularly administered to the eye.

In some embodiments, the ophthalmic formulation can be delivered to the ocular surface, interconnecting innervation, conjuncitva, lacrimal glands, or meibomian glands. It is envisioned that effective treatment can encompass administering the polypeptides of the present disclosure via oral administration, topical administration, via injection, intranasally, rectally, transdermally, via an impregnated or coated device such as an ocular insert or implant, or iontophoretically, amongst other routes of administration.

In some embodiments, the ophthalmic formulation is formulated for injection. For administration via injection, the ophthalmic formulation can be injected intramuscularly, intra-arterially, subcutaneously, or intravenously. A pump mechanism may be employed to administer the ophthalmic formulation over a preselected period. For some embodiments of the invention, it is desirable to deliver drug locally, thus injections may be made periocularly, intraocularly, subconjunctively, retrobulbarly, or intercamerally.

In some embodiments, the ophthalmic formulation may be administered to the ocular surface via a pump-catheter system, or released from within a continuous or selective release device such as, e.g., membranes such as, but not limited to, those employed in the Ocusert™ System (Alza Corp, Palo Alto, CA). The ophthalmic formulations can be incorporated within, carried by or attached to contact lenses which are then worn by the subject. The ophthalmic formulations can be sprayed onto ocular surface.

In some embodiments, the ophthalmic formulation comprises from about 0.001 M to about 100 M of the polypeptides of the present disclosure. In some embodiments, the ophthalmic formulation comprises from about 0.01 M to about 20 M of the polypeptides of the present disclosure. In some embodiments, the ophthalmic formulation comprises from about 0.1 M to about 5 M of the polypeptides of the present disclosure. In some embodiments, the ophthalmic formulation comprises from about 0.2 M to about 3 M of the polypeptides of the present disclosure. In some embodiments, the ophthalmic formulation comprises from about 0.1 M to about 10 M of the polypeptides of the present disclosure. In some embodiments, the ophthalmic formulation comprises from about 1 M to about 5 M of the polypeptides of the present disclosure. In some embodiments, the ophthalmic formulation comprises from about 1 M to about 10 M of the polypeptides of the present disclosure. In some embodiments, the ophthalmic formulation comprises from about 5 M to about 10 M of the polypeptides of the present disclosure. In some embodiments, the ophthalmic formulation comprises from about 10 M to about 50 M of the polypeptides of the present disclosure. In some embodiments, the ophthalmic formulation comprises from about 20 M to about 50 M of the polypeptides of the present disclosure. In some embodiments, the ophthalmic formulation comprises from about 5 M to about 50 M of the polypeptides of the present disclosure. In some embodiments, the ophthalmic formulation comprises from about 1 M to about 50 M of the polypeptides of the present disclosure. In some embodiments, the ophthalmic formulation comprises from about 1 M to about 20 M of the polypeptides of the present disclosure. In some embodiments, the ophthalmic formulation comprises from about 5 M to about 20 M of the polypeptides of the present disclosure. In some embodiments, the ophthalmic formulation comprises from about 10 M to about 20 M of the polypeptides of the present disclosure. In some embodiments, the ophthalmic formulation comprises higher than about 0.001, 0.05, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 M of the polypeptides of the present disclosure. In some embodiments, the ophthalmic formulation comprises lower than about 100, 90, 80, 70, 60, 50, 40, 30, 20, 25, 20, 19, 18, 17, 16, 15, 14, 13, 12, 11, 10, 9, 8, 7, 6, 5, or 4 M of the polypeptides of the present disclosure.

In some embodiments, the ophthalmic formulation can be formulated into a dosage form from about 0.01 to about 10 ml for use once or multiple times. In some embodiments, the ophthalmic formulation is formulated into a unit dosage form to provide a total daily dosage of from about 0.01 to about 2 ml. In some embodiments, the ophthalmic formulation can be formulated into a unit dosage form to provide a total weekly dosage of from about 1 ml to about 5 ml. In some embodiments, the ophthalmic formulation can be formulated into a unit dosage form to provide a total monthly dosage of from about 1 ml to about 20 ml.

In some embodiments, the ophthalmic formulation is an ophthalmic formulation for use in human.

In some embodiments, the ophthalmic formulation is a veterinary ophthalmic formulation.

In some embodiments, the methods of the present disclosure comprise administrating an effective amount of the polypeptides of the present disclosure to one affected eye or eye tissue of the subject. In some embodiments, the methods of the present disclosure comprise administrating an effective amount of the polypeptides of the present disclosure to both eyes or eye tissues of the subject. In some embodiments, the effective amount of the polypeptides of the present disclosure is from about 0.1 g to about 100 g per eye, or from about 0.1 g to about 50 g per eye, or from about 0.1 g to about 20 g per eye, or from about 0.1 g to about 10 g per eye, or from about 0.5 g to about 50 g per eye, or from about 0.5 g to about 20 g per eye, or from about 0.5 g to about 10 g per eye, or from about 1 g to about 10 g per eye.

In some embodiments, the dosage for one eye of the subject can be about 1 to about 5 drops of the ophthalmic formulation. In some embodiments, the dosage for one eye of the subject can be 1 drop of the ophthalmic formulation. In some embodiments, the dosage for one eye of the subject can be 2 drops of the ophthalmic formulation. In some embodiments, the dosage for one eye of the subject can be 3 drops of the ophthalmic formulation. In some embodiments, the dosage for one eye of the subject can be 4 drops of the ophthalmic formulation. In some embodiments, the dosage for one eye of the subject can be 5 drops of the ophthalmic formulation. In some embodiments, each drop corresponds to about 10 μL to about 150 μL. In some embodiments, each drop corresponds to about 20 μL to about 70 μL.

In some embodiments, the methods of the present disclosure comprise administrating an effective amount of the ophthalmic formulation in each eye of the subject once daily. In some embodiments, the methods of the present disclosure comprise administrating an effective amount of the ophthalmic formulation in each eye of the subject twice daily. In some embodiments, the methods of the present disclosure comprise administrating an effective amount of the ophthalmic formulation in each eye of the subject three or more times daily. In some embodiments, the methods of the present disclosure comprise administrating an effective amount of the ophthalmic formulation in each eye of the subject every two days.

In some embodiments, the methods of the present disclosure comprise administrating an effective amount of the ophthalmic formulation in each eye of the subject every three days. In some embodiments, the methods of the present disclosure comprise administrating an effective amount of the ophthalmic formulation in each eye of the subject weekly.

In some embodiments, the methods of the present disclosure comprise administrating one or more drops of the ophthalmic formulation in each eye of the subject daily. In some embodiments, the methods of the present disclosure comprise administrating one to more drops of the ophthalmic formulation in each eye of the subject 2, 3, 4, 8, 12, 18 or 24 times daily. In some embodiments, the methods of the present disclosure comprise administrating one or more drops of the ophthalmic formulation in each eye of the subject every two days.

In some embodiments, the polypeptides of the present disclosure or the ophthalmic formulation may be placed in a kit. In some embodiments, the kit comprises one or more of the polypeptides of the present disclosure or the ophthalmic formulation, and an instruction for using the kit.

The kits can comprise one or more containers that contain one or more of the polypeptides of the present disclosure or the ophthalmic formulation. The polypeptides of the present disclosure can be present in the container as a prepared formulation, or alternatively, the polypeptides of the present disclosure can be unformulated. In some embodiments, the kit can include unformulated polypeptides of the present disclosure in a container that is separate from the pharmaceutically acceptable excipients. Prior to use, the polypeptides of the present disclosure is diluted or otherwise mixed with the pharmaceutically acceptable excipients.

In some embodiments, the kit also comprises instructions which describe the method for administering the polypeptides of the present disclosure or the ophthalmic formulation. In some embodiments, the instructions also describe the procedure for mixing the polypeptides of the present disclosure contained in the kit with pharmaceutically acceptable excipients.

### Preparation of the Polypeptide

The polypeptides of the present disclosure, e.g., an artificial polypeptide of formula (I), (II), (III), (IV), (V), (VI) or (VII), or a mutant of an artificial polypeptide of formula (I), (II), (III), (IV) or (V), or a polypeptide having at least 70% identity (e.g., at least 80% identity) with any one selected from SEQ ID NO: 28, SEQ ID NOs: 57 and 62-74 or a fragment or variant thereof can be prepared by any suitable method, including but not limited to molecular cloning techniques and synthetic procedures. Standard molecular cloning techniques are well known in the art and are described by Sambrook, J., Fritsch, E. F. and Maniatis, T. Molecular Cloning: A Laboratory Manual; Cold Spring Harbor Laboratory Press: Cold Spring Harbor, (1989) (Maniatis) and by T. J. Silhavy, M. L. Bennan, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, pub. by Greene Publishing Assoc. and Wiley-Interscience (1987).

In some embodiments, the polypeptides of the present disclosure are prepared by reference to the fermentation-based manufacturing method as disclosed in Chinese patent application No. 201711320516.4 (Publication No.: CN109913483A), which is herein incorporated by reference in its entirety. In some embodiments, the method for preparing the polypeptides of the present disclosure comprises the steps of: integrating a target gene fragment into an expression plasmid by means of genetic engineering, with the integrated target gene fragment comprising at least one purification tag; transforming the expression plasmid into a corresponding expression host to construct a recombinant engineered cell which highly expresses the target polypeptide; subjecting the recombinant engineered cell to fermentation, induced expression, and then crude purification to obtain a crude polypeptide; subjecting the crude polypeptide to refined purification to obtain highly purified polypeptide.

In some embodiments, the target gene fragment is any one selected from the group consisting of gene fragments which are capable of encoding the polypeptides of the present disclosure, e.g., an artificial polypeptide of formula (I), (II), (III), (IV), (V), (VI) or (VII), or a mutant of an artificial polypeptide of formula (I), (II), (III), (IV) or (V), or a polypeptide having at least 70% identity (e.g., at least 80% identity) with any one selected from SEQ ID NO: 28, SEQ ID NOs: 57 and 62-74 or a fragment or variant thereof.

In some embodiments, the target gene fragment is a gene fragment which is capable of encoding an artificial polypeptide of formula (I), (II), (III), (IV), (V), (VI) or (VII). In some embodiments, the target gene fragment is a gene fragment which is capable of encoding a mutant of an artificial polypeptide of formula (I), (II), (III), (IV) or (V). In some embodiments, the target gene fragment is a gene fragment which is capable of encoding a polypeptide having at least 70% identity (e.g., at least 80% identity) with any one selected from SEQ ID NO: 28, SEQ ID NOs: 57 and 62-74 or a fragment or variant thereof. The target gene fragment can be prepared by any suitable method, including but not limited to enzymatic synthesis, i.e., using RNA as a template to synthesize cDNA by reverse transcription, and chemical synthesis method, i.e., using a chemical method or chemical method combined with enzymatic method to synthesize target gene. The preparation of target gene fragment may also be commercially conducted by a contract research organization (CRO) in case that the sequence of target gene fragment is provided.

In some embodiments, the purification tag is a ST sequence tag (an amino acid sequence that helps the polypeptides of the present disclosure to form inclusion bodies) or a His tag.

In some embodiments, the expression host is a host cell. The host cell includes but is not limited to an individual cell, cell culture, or cell line. In some embodiments, the host cells include progeny of a single host cell. In some embodiments, a host cell can be transfected with a heterologous sequence including vectors encoding the polypeptides of the present disclosure. In some embodiments, said host cells may be prokaryotic cells, such as bacterial cells. In some embodiments, said host cells may be eukaryotic cells, such as yeast cells, animal cells, insect cells, plant cells and the like.

Examples of bacterial host cells that can be used to produce the polypeptide or the mutant of the present disclosure include microorganisms belonging to the genus Escherichia, Serratia, Bacillus, Brevibacterium, Corynebacterium, Microbacterium, Pseudomonas and the like. For example, bacterial host cells may include, but not be limited to, Escherichia coli XL1-Blue, XL2-Blue, DH1, MC1000, KY3276, W1485, JM109, HB101, No. 49, i W3110, NY49, G1698, BL21, or TB1. Other bacterial host cells may include, but not be limited to, Serratia ficaria, Serratia fonticola, Serratia liquefaciens, Serratia marcescens, Bacillus subtilis, Bacillus amyloliquefaciens, Brevibacterium ammoniagenes, Brevibacterium immariophilum ATCC 14068, Brevibacterium saccharolyticum ATCC 14066, Brevibacterium flavum ATCC 14067, Brevibacterium lactofermentum ATCC 13869, Corynebacterium glutamicum ATCC 13032, Corynebacterium glutamicum ATCC 13869, Corynebacterium acetoacidophilum ATCC 13870, Microbacterium ammoniaphilum ATCC 15354, Pseudomonas putida, Pseudomonas sp. D-0110 and the like.

Examples of yeast cells that can be used to produce the polypeptides of the present disclosure include microorganisms belonging to the genus Kluyveromyces, Trichosporon, Saccharomyces, Schizosaccharomyces, Schwanniomyces, Pichia, Candida and the like, such as Saccharomyces cerevisiae, Schizosaccharomyces pombe, Kluyveromyces lactis, Trichosporon pullulans, Schwanniomyces alluvius, Candida utilis and the like.

Examples of animal cells that can be used to produce the polypeptides of the present disclosure include mammalian cells, for example, Chinese hamster ovary cells (CHO) or monkey cells, such as COS cells, HepG2 cells, A549 cells, and any other cells available through ATCC or other depositories.

In some embodiments, the expression host is an Escherichia Coli host cell. In some embodiments, the formula of the fermentation medium used in the fermentation process is: yeast extract powder 10-50 g/L, peptone 10-30 g/L, ammonium sulfate 2-10 g/L, sodium chloride 2-10 g/L, potassium dihydrogen phosphate 0-10 g/L, dipotassium hydrogen phosphate 2-15 g/L, defoamer 0.01-0.1% (v/v), FeSO₄·7H₂O 0-0.1 g/L, ZnSO₄·7H₂O 0-0.02 g/L, CUSO₄·5H₂O 0-0.1 g/L, MnSO₄·5H₂O 0-0.05 g/L, CaCl₂·7H₂O 0-0.01 g/L, CoCl₂·6H₂O 0-0.01 g/L, Na₂MoO₄·2H₂O 0-0.01 g/L, H₃BO₃ 0-0.0005 g/L, and Biotin 0-0.005 g/L. In some embodiments, the fermentation process is performed at 37° C.

In some embodiments, the induced expression is realized by addition of isopropyl-beta-D-thiogalactopyranoside (IPTG) during the fermentation process. In some embodiments, the IPTG is added 0.5, 1, 2, 3, 4 or 5 hours after the initiation of the fermentation process. In some embodiments, the IPTG is added at a final concentration of 0.25, 0.5, 1, 2 or 4 mM. In some embodiments, the induced expression is performed at 37° C.

The crude purification of the present disclosure is a process during which the culture produced by the fermentation process is preliminary treated. In some embodiments, the crude purification comprises the steps of collecting the cells and isolating the inclusion body proteins and/or cytoplasmic proteins after lysing the cells; subjecting the inclusion body proteins and/or cytoplasmic proteins to denaturation, renaturation and enzymatic digestion to obtain a crude product containing the crude polypeptide. In some embodiments, the crude purification comprises the steps of collecting the culture medium, removing the cells and impurities, and obtaining the supernatant, i.e., a crude product containing the crude polypeptide.

The refined purification of the present disclosure is a process during which the crude product containing the crude polypeptide is purified with a chromatographic method. Examples of chromatographic methods that can be used to purify the polypeptides of the present disclosure include ion exchange chromatography with a strong anion exchange resin, a weak anion exchange resin or a multimodal anion exchange resin; affinity chromatography; reversed phase chromatography with reversed phase packing materials; molecular sieve chromatography with size exclusion packing materials; and hydrophobic chromatography with hydrophobic packing materials.

More detailed information for preparing the polypeptides of the present disclosure may be found throughout of the disclosure of CN109913483A, e.g., Examples 1-4.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EXAMPLES

The present disclosure will now be described with reference to the following Example, which is intended to illustrate, but not limit, the invention.

Unless otherwise specified, if the specific conditions are not indicated in the Example, it is carried out in accordance with the conventional conditions or the conditions suggested by the manufacturer. The reagents or instruments used without the manufacturer's indication are conventional products that commercially available. Those skilled in the art would understand that the Example described herein is not intended to limit the scope of the invention as claimed.

### Example 1: Effects of the Polypeptides of the Present Disclosure on the Treatment of Allergic Conjunctivitis Induced by Chicken Ovalbumin (OVA) in New Zealand Rabbits

The purpose of this Example is to investigate the effects of the polypeptides of the present disclosure on the treatment of allergic conjunctivitis. To this end, chicken ovalbumin (OVA) is used as an allergen to induce a specific immune response in New Zealand rabbits, resulting in an allergic conjunctivitis manifestation in the rabbits' eyes. Then the polypeptides of the present disclosure are administrated to the rabbits to study whether the polypeptides have certain therapeutic effects on allergic conjunctivitis in rabbits.

### Animals

42 New Zealand rabbits weighting about 2.0-3.5 kg (6-8 weeks old) were used. Among them, 30 rabbits, including both male and female, were selected to enroll in the study.

### Modeling

In this study, subcutaneous injections of 200 μg of OVA were performed once every five days. Meanwhile, 80 μg/μl of OVA was administered to each eye per day for five days in the first week; after a one-day drug holiday, the administrations of OVA to eyes were continued for three days, then another one-day drug holiday was performed in order to strengthen the sensitization. The rabbits started to display symptoms such as edema of conjunctiva, hyperemia of conjunctiva, edema of eyelids, hyperemia of eyelids, visible damage of cornea, mucous discharge, tearing and difficulty in opening eyes from about D11-D15 after modeling. This fact indicated that the modelling of allergic conjunctivitis was successful.

### Grouping and Administration

Twelve days after modeling, after being verified by slit lamp photography and eye irritation test, the rabbits were randomly divided into the following 3 groups, with 10 animals in each group. The 20 eyes of the rabbits were subjected to the operations as set forth in Table 1.

TABLE 1
A summary of the experimental details of grouping and administration

| | | Source of the | | Ophthalmic administration or treatment | |
|---|---|---|---|---|---|
| Group | Treatment | animals | N | Left eye | Right eye |
| 1 | Blank control | Blank control | 10 | Subcutaneous | Subcutaneous |
| | | group | | injections of OVA + | injections of OVA + |
| | | | | ophthalmic | ophthalmic |
| | | | | administrations of | administrations of |
| | | | | OVA | OVA |
| 2 | Test | Blank control | 10 | Subcutaneous | Subcutaneous |
| | substance | group | | injections of OVA + | injections of OVA + |
| | group 1 of | | | ophthalmic | ophthalmic |
| | allergic | | | administrations of | administrations of |
| | conjunctivitis | | | OVA + treatments | OVA₊ treatments |
| | | | | with test | with test substance |
| | | | | substance*(tid) | (tid) |
| 3 | Test | Blank control | 10 | Subcutaneous | Subcutaneous |
| | substance | group | | injections of OVA + | injections of OVA + |
| | group 2 of | | | ophthalmic | ophthalmic |
| | allergic | | | administrations of | administrations of |
| | conjunctivitis | | | OVA #+ treatments | OVA + treatments |
| | | | | with test substance | with test substance |
| | | | | (tid) | (tid) |

*the final dosing design needs to be determined according to the way of administration of the test substance(s)
#: subcutaneous injections of OVA + ophthalmic administrations of OVA were stopped on D 19 after modeling.

In brief, administrations were performed according to the operations as set forth in Table 1, started on D12 after modeling (depending on the specific situations) and continued for 2 weeks. Among them, one week is for administration while modeling, and one week is for administration during the recovery period after termination of modeling.

### Ocular Examination Items

The following ocular examination items were performed according to the detailed description as set forth in Table 2.

TABLE 2
A summary of the detailed description of ocular examination items

| Ocular examination items | Detailed description |
|---|---|
| Clinical observation | Once a day, the animals' death or near-death situation, mental state, |
| | behavioral activity, eating situation, fecal property, etc. were observed |
| | by the side of the cages |
| Ocular surface examination | The ocular surface examinations were performed on the transferred |
| | animals before the adaptation period, and the New Zealand rabbits with |
| | normal ocular surface were enrolled in the experimental groups/after |
| | establishing the allergic conjunctivitis model, the ocular surfaces of the |
| | rabbits were tested, and the rabbits that did not meet the requirements |
| | were excluded from the next step of the experiment |
| Body weight | Body weight was measured for all rabbits when the reserved rabbits |
| | were transferred in, before modeling, and before each ocular |
| | examination |
| General ocular examination | General ocular examination was performed once for all rabbits during |
| (with direct ophthalmoscope, | screening, before modeling, after modeling (D 6, D 9, D 10, D 12, D 13, |
| slit lamp, etc.) | D 15), before administration (D-10, D-12), 0 h and 30 min after |
| | administration (D 1), D 2 after administration, D 3 after administration, |
| | D 5 after administration, D 7 after administration and D 14 after |
| | administration/the changes in conjunctiva, cornea, anterior chamber, |
| | iris, pupil, vitreous body and fundus of both eyes of the rabbits were |
| | investigated/if obvious abnormalities were observed in general ocular |
| | examination, photos would be taken with a slit lamp, and then |
| | additional detection indices would be examined, the frequency of index |
| | detection would be increased, and/or inflammation score would be |
| | measured, all of which were at the discretion of the person in charge of |
| | the study |
| Detection of recovery from | Detection of recovery from corneal injury was performed once for all |
| corneal injury | New Zealand rabbits before modeling, before administration (D-10, D- |
| | 12), 0 h after administration (D 1), |
| | D 2 after administration, D 3 after administration, D 5 after |
| | administration, D 7 after administration and D 14 after administration/ |
| | the corneas were stained with sodium fluorescein, observed with cobalt |
| | blue light under a slit lamp and were scored |
| Tissue and blood collection | On D 15 after administration (D 27 after modeling), the tested rabbits |
| | were euthanized, and the following collections were carried out for all |
| | animals from each group being tested, respectively: corneas and |
| | conjunctivae from both eyes/if test substances were found to be |
| | effective under clinical ocular examination, the corneas and |
| | conjunctivae were separated from the left eyeballs and quick-frozen at |
| | the end of euthanasia, and the right eyeballs were fixed/Blood was |
| | collected on D 19 after modeling |

### Statistical Analysis

Inflammation scores, collected pathological tissues and results of detection of recovery from corneal injury obtained in the study were subject to statistical analysis.

### Example 2: Effects of the Polypeptides of the Present Disclosure on the Treatment of Allergic Conjunctivitis Induced by Chicken Ovalbumin (OVA) in Mice

The purpose of this example is to investigate the effects of the polypeptides of the present disclosure on the treatment of allergic conjunctivitis. To this end, chicken ovalbumin (OVA) was used as an allergen to induce a specific immune response in mice, resulting in an allergic conjunctivitis manifestation in the eyes of mice after challenging. Then the polypeptides of the present disclosure were administrated to the mice to study whether the polypeptides have certain therapeutic effects on allergic conjunctivitis in mice.

### Modeling

Female 6-week-old mice were selected to enroll in the study. In this study, the mice were subcutaneously injected with OVA on Day 0) to induce sensitization. The sensitization procedure was repeated on Day 5 to enhance the allergic reaction. OVA was administered to each eye as a challenge every other day from Day 10 to 14. The mice displayed symptoms such as edema of conjunctiva, hyperemia of conjunctiva, edema of eyelids, hyperemia of eyelids, visible damage of cornea, mucous discharge, tearing and difficulty in opening eyes after challenging. This fact indicated that the modelling of allergic conjunctivitis was successful.

### Grouping and Administration

The mice were randomly divided into the following 3 groups (N=10 per group). The eyes of the mice were subjected to the operations as set forth in Table 3.

TABLE 3
A summary of the experimental details of grouping and administration

| Group | Treatment | N | Ophthalmic administration or treatment |
|---|---|---|---|
| 1 | Blank control | 10 | / |
| | Test substance | | Treatment with one drop (approximately 10 μl) of SEQ |
| 2 | group 1 of allergic | 10 | ID NO: 29 (formulated as eye drops (60 μg/ml)) per eye |
| | conjunctivitis | | each time, tid |
| 3 | Test substance | 10 | Treatment with one drop (approximately 10 μl) of SEQ ID |
| | group 2 of allergic | | NO: 89 (formulated as eye drops (60 μg/ml)) per eye each |
| | conjunctivitis | | time, tid |

In brief, administrations were performed according to the operations as set forth in Table 3, started on D9 and continued for 1 week.

### Ocular Examination Items

The mice were subjected to general ocular examination once daily during administration period. Specifically, conjunctival edema, mucous discharge, and conjunctival redness were graded from 0 to 4 (0: absent; 1: minimal; 2: mild; 3: moderate; 4: severe) based on the criteria set forth in Merayo-Lloves J, Zhao T Z, Dutt J E, Foster C S. A new murine model of allergic conjunctivitis and effectiveness of nedocromil sodium. J Allergy Clin Immunol 1996; 97:1129-40 (see, e.g., Table 1). Inflammation scores of the mice, calculated as the sum of the 3 independent scores, were measured. In addition, the scratching times were evaluated after challenging. The scratching response was defined as rapid movements of the hind paws that were precisely directed toward the eye. After the last administration, the mice were euthanized, and the tissues and blood were collected for further study (data not shown).

### Results

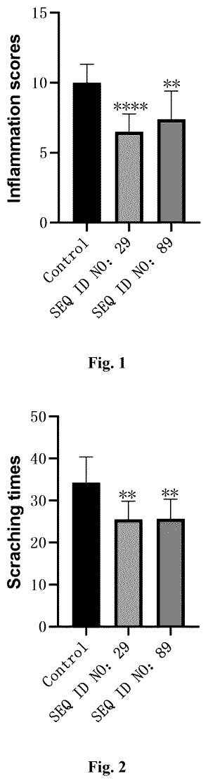

FIG. 1 shows the inflammation scores of the mice from respective groups on Day 14. Inflammation scores indicate the degree of inflammation in the eyes, which has been used to assess allergic conjunctivitis.

It was found that, the inflammation scores of the SEQ ID NO: 29 group and SEQ ID NO: 89 group were both significantly lower than those of the control group (SEQ ID NO: 29, p<0.0001; SEQ ID NO: 89, p<0.01). The results showed that the polypeptides of the present disclosure can significantly improve the symptoms of allergic conjunctivitis.

FIG. 2 shows the scratching times of the mice from respective groups on Day 14. Scratching times indicate the severity of allergic conjunctivitis symptoms. It was found that, scratching times were significantly lower in the SEQ ID NO: 29 group and SEQ ID NO: 89 group compared to the control group (SEQ ID NO: 29, p<0.01; SEQ ID NO: 89, p<0.01), indicating that treatments with the polypeptides of the present disclosure can significantly reduce the scratching times. The results showed that the polypeptides of the present disclosure have definite therapeutic effect on allergic conjunctivitis.

SEQUENCE LISTING

| SEQ ID | |
|---|---|
| NO | Sequences |
| 1 | SEKKQPVDLGLLEEDDEFEEFPAEDWAGLDEDEDAHVWEDNWDDDNVEDDFSNQLRA |
| 2 | TVLLMILVCETPYGCYVLHQKGRMCSAFLCC |
| 3 | TVLLMILVAETPYGAYVLHQKGRMASAFLAA |
| 4 | TVLLMILVCETPYGCYVLHQKGRMCSAFLCA |
| 5 | TVLLMILVVETPYGVYVLHQKGRMVSAFLVV |
| 6 | TVLLMILVAETPYGAYVLHQKGRMVSAFLVV |
| 7 | TVLLMILVAETPYGVYVLHQKGRMASAFLVV |
| 8 | TVLLMILVAETPYGVYVLHQKGRMVSAFLAV |
| 9 | VLLMILVAETPYGVYVLHQKGRMVSAFLVA |
| 10 | TVLLMILVVETPYGAYVLHQKGRMASAFLVV |
| 11 | TVLLMILVVETPYGAYVLHQKGRMVSAFLAV |
| 12 | TVLLMILVVETPYGAYVLHQKGRMVSAFLVA |
| 13 | TVLLMILVVETPYGVYVLHQKGRMASAFLAV |
| 14 | TVLLMILVVETPYGVYVLHQKGRMASAFLVA |
| 15 | TVLLMILVVETPYGVYVLHQKGRMVSAFLAA |
| 16 | TVLLVILVAETPYGAYVLHQKGRVASAFLAA |
| 17 | GLCALCLMVRQPCFCLEIYMGVKHTCLYVST |
| 18 | SLLCVFPTIECCRYKMGLLYHCVACTQGMLV |
| 19 | SEFEDANKEQGEVPNDDVSDELPKLLDDDEDELRGALFFQWHDWEEAEDNDWDVEDA |
| 20 | SELDFWDNWKDRQADLPAASDDEDFENDLAEVDHEDPLVDGNEWDGDEVEFELEEQK |
| 21 | SDADEFDDDNSVWGWEDDEVEPNDAFQPLGDVRANEELDEKDHKLDEEADLQWEFEL |
| 22 | SQWAPLDHLESDEWGAFEDEDFNNGDVEELDPEERDDEDLFQKDVLVAKADDEEWDN |
| 23 | STKKQPVDLGLLETDSEFTEFPATSWAGLSESESAHVWTSNWDSSNVTSDFSNQLRA |
| 24 | STKKQPVDLGLLETDDEFEEFPATSWAGLSEDESAHVWESNWDDSNVTSDFSNQLRA |
| 25 | STKKQPVDLGLLEEDDEFEEFPAESWAGLDEDESAHVWEDNWDDDNVTSDFSNQLRA |
| 26 | SEKKQPVDLGLLEEDDEFEEFPAEDWAGLDEDESAHVWEDNWDDDNVTSDFSNQLRA |
| 27 | SEKKQPVDLGLLEEDDEFEEFPAEDWAGLDEDEDAHVWEDNWDDDNVTSDFSNQLRA |
| 28 | SEKKQPVDLGLLEEDDEFEEFPAEDWAGLDEDEDAHVWEDNWDDDNVEDDFSNQLRAT |
| | VLLMILVCETPYGCYVLHQKGRMCSAFLCC |
| 29 | SEKKQPVDLGLLEEDDEFEEFPAEDWAGLDEDEDAHVWEDNWDDDNVEDDFSNQLRAT |
| | VLLMILVAETPYGAYVLHQKGRMASAFLAA |
| 30 | SEKKQPVDLGLLEEDDEFEEFPAEDWAGLDEDEDAHVWEDNWDDDNVEDDFSNQLRAT |
| | VLLMILVCETPYGCYVLHQKGRMCSAFLCA |
| 31 | SEKKQPVDLGLLEEDDEFEEFPAEDWAGLDEDEDAHVWEDNWDDDNVEDDFSNQLRAT |
| | VLLMILVVETPYGVYVLHQKGRMVSAFLVV |
| 32 | SEKKQPVDLGLLEEDDEFEEFPAEDWAGLDEDEDAHVWEDNWDDDNVEDDFSNQLRAT |
| | VLLMILVAETPYGAYVLHQKGRMVSAFLVV |
| 33 | SEKKQPVDLGLLEEDDEFEEFPAEDWAGLDEDEDAHVWEDNWDDDNVEDDFSNQLRAT |
| | VLLMILVAETPYGVYVLHQKGRMASAFLVV |
| 34 | SEKKQPVDLGLLEEDDEFEEFPAEDWAGLDEDEDAHVWEDNWDDDNVEDDFSNQLRAT |
| | VLLMILVAETPYGVYVLHQKGRMVSAFLAV |
| 35 | SEKKQPVDLGLLEEDDEFEEFPAEDWAGLDEDEDAHVWEDNWDDDNVEDDFSNQLRAT |
| | VLLMILVAETPYGVYVLHQKGRMVSAFLVA |
| 36 | SEKKQPVDLGLLEEDDEFEEFPAEDWAGLDEDEDAHVWEDNWDDDNVEDDFSNQLRAT |
| | VLLMILVVETPYGAYVLHQKGRMASAFLVV |
| 37 | SEKKQPVDLGLLEEDDEFEEFPAEDWAGLDEDEDAHVWEDNWDDDNVEDDFSNQLRAT |
| | VLLMILVVETPYGAYVLHQKGRMVSAFLAV |
| 38 | SEKKQPVDLGLLEEDDEFEEFPAEDWAGLDEDEDAHVWEDNWDDDNVEDDFSNQLRAT |
| | VLLMILVVETPYGAYVLHQKGRMVSAFLVA |
| 39 | SEKKQPVDLGLLEEDDEFEEFPAEDWAGLDEDEDAHVWEDNWDDDNVEDDFSNQLRAT |
| | VLLMILVVETPYGVYVLHQKGRMASAFLAV |
| 40 | SEKKQPVDLGLLEEDDEFEEFPAEDWAGLDEDEDAHVWEDNWDDDNVEDDFSNQLRAT |
| | VLLMILVVETPYGVYVLHQKGRMASAFLVA |
| 41 | SEKKQPVDLGLLEEDDEFEEFPAEDWAGLDEDEDAHVWEDNWDDDNVEDDFSNQLRAT |
| | VLLMILVVETPYGVYVLHQKGRMVSAFLAA |
| 42 | SEKKQPVDLGLLEEDDEFEEFPAEDWAGLDEDEDAHVWEDNWDDDNVEDDFSNQLRAG |
| | LCALCLMVRQPCFCLEIYMGVKHTCLYVST |
| 43 | SEKKQPVDLGLLEEDDEFEEFPAEDWAGLDEDEDAHVWEDNWDDDNVEDDFSNQLRAS |
| | LLCVFPTIECCRYKMGLLYHCVACTQGMLV |
| 44 | SEKKQPVDLGLLEEDDEFEEFPAEDWAGLDEDEDAHVWEDNWDDDNVEDDFSNQLRAT |
| | VLLVILVAETPYGAYVLHQKGRVASAFLAA |
| 45 | SEFEDANKEQGEVPNDDVSDELPKLLDDDEDELRGALFFQWHDWEEAEDNDWDVEDAT |
| | VLLMILVCETPYGCYVLHQKGRMCSAFLCC |
| 46 | SELDFWDNWKDRQADLPAASDDEDFENDLAEVDHEDPLVDGNEWDGDEVEFELEEQKT |
| | VLLMILVCETPYGCYVLHQKGRMCSAFLCC |
| 47 | SDADEFDDDNSVWGWEDDEVEPNDAFQPLGDVRANEELDEKDHKLDEEADLQWEFELT |
| | VLLMILVCETPYGCYVLHQKGRMCSAFLCC |
| 48 | SQWAPLDHLESDEWGAFEDEDFNNGDVEELDPEERDDEDLFQKDVLVAKADDEEWDNT |
| | VLLMILVCETPYGCYVLHQKGRMCSAFLCC |
| 49 | STKKQPVDLGLLETDSEFTEFPATSWAGLSESESAHVWTSNWDSSNVTSDFSNQLRATVL |
| | LMILVCETPYGCYVLHQKGRMCSAFLCC |
| 50 | STKKQPVDLGLLETDDEFEEFPATSWAGLSEDESAHVWESNWDDSNVTSDFSNQLRATVL |
| | LMILVCETPYGCYVLHQKGRMCSAFLCC |
| 51 | STKKQPVDLGLLEEDDEFEEFPAESWAGLDEDESAHVWEDNWDDDNVTSDFSNQLRATV |
| | LLMILVCETPYGCYVLHQKGRMCSAFLCC |
| 52 | SEKKQPVDLGLLEEDDEFEEFPAEDWAGLDEDESAHVWEDNWDDDNVTSDFSNQLRAT |
| | VLLMILVCETPYGCYVLHQKGRMCSAFLCC |
| 53 | SEKKQPVDLGLLEEDDEFEEFPAEDWAGLDEDEDAHVWEDNWDDDNVTSDFSNQLRAT |
| | VLLMILVCETPYGCYVLHQKGRMCSAFLCC |
| 54 | SEKKQPVDLGLLEEDDEFEEFPAEDWAGLDEDEDAHVWEDNWDDDNVTSDFSNQLRAT |
| | VLLMILVCETPYGCYVLHQKGRMCS |
| 55 | SEKKQPVDLGLLEEDDEFEEFPAEDWAGLDEDEDAHVWEDNWDDDNVTSDFSNQLRAT |
| | VLLMILVCETPYGCYVLHQK |
| 56 | SEKKQPVDLGLLEEDDEFEEFPAEDWAGLDEDEDAHVWEDNWDDDNVTSDFSNQLRAT |
| | VLLMILVCET |
| 57 | SEKKQPVDLGLLEEDDEFEEFPAEDWAGLDEDEDAHVWEDNWDDDNVTSDFSNQLRAT |
| | VLLMI |
| 58 | SEKKQPVDLGLLEEDDEFEEFPAEDWAGLDEDEDAHVWEDNWDDDNVTSDFSNQLRA |
| 59 | SEKKQPVDLGLLEEDDEFEEFPAEDWAGLDEDEDAHVWEDNWDDDNVTSDFSNQLRA |
| 60 | SEKKQPVDLGLLEEDDEFEEFPAEDWAGLDEDEDAHVWEDNWDDDNVTSDFSNQLRA |
| 61 | SEKKQPVDLGLLEEDDEFEEFPAEDWAGLDEDEDAHVWEDNWDDDNVTSDFSNQLRA |
| 62 | SEKKQPVDLGLLEEDDEFEEFPAEDWAGLDEDEDAHVWEDNWDDDNVEDDFSNQLRAE |
| | LEKHGYKMETS |
| 63 | MSEKKQPVDLGLLEEDDEFEEFPAEDWAGLDEDEDAHVWEDNWDDDNVEDDFSNQLRA |
| | TVLLMILVCETPYGCYVLHQKGRMCSAFLCC |
| 64 | MSEKKQPVDLGLLEEDDEFEEFPAEDWAGLDEDEDAHVWEDNWDDDNVEDDFSNQLRA |
| | TVLLMILVCETPYGCYVLHQKGRMCSAFLCC |
| 65 | MSEKKQPVDLGLLEEDDEFEEFPAEDWAGLDEDEDAHVWEDNWDDDNVEDDFSNQLRV |
| | TVLLMILVCETLYGCYVLHQKGRMCSAFLCC |
| 66 | MSEKKQPVDLGLLEEDDEFEEFPAEDWAGLDEDEDAHVWEDNWDDDNVEDDFSNQLRA |
| | TILLMILVCETPYGCYVLHQKGRMCSAFLCC |
| 67 | MSEKKQPVDLGLLEEDDEFEEFPAEDWAGLDEDEDAHVWEDNWDDDNVEDDFSNQLRA |
| | TVLLMVLVCETPYGCYVLHQKERMCSAFLCC |
| 68 | MSEKKQPVDLGLLEEDDEFEEFPAEDWAGLDEDEDAHVWEDNWDDDNVEDDFSNQLRA |
| | TVLLMIKVYETPYGCYILHQKGRMCSAFLCC |
| 69 | MSEKKQPVDLGLLEEDDEFEEFPAEDWAGLDEDEDAHVWEDNWDDDNVEDDFSNQLRA |
| | TVLLMIKVYETPYGCYILHQKGRMCSAFLCC |
| 70 | MSEKKQPVDLGLLEEDDEFEEFPAEDWAGLDEDEDAHVWEDNWDDDNVEDDFSNQLRA |
| | TVLLMIKVYETPYGCYILHQKGRMCSAFLCC |
| 71 | MSEKKQPVDLGLLEEDDEFEEFPAEDWAGLDEDEDAHVWEDNWDDDNVEDDFSNQLRA |
| | TVLLMKKVYETPYGCYILHQKGRMCSAFLCC |
| 72 | MSEKKQPVDLGLLEEDDEFEEFPAEDWAGLDEDEDAHVWEDNWDDDNVEDDFSNQLRA |
| | TVLLMIKVYETPYGCYILHQKGRMCSAFLCC |
| 73 | MSEKKQPVDLGLLEEDDEFEEFPAEDWAGLDEDEDAHVWEDNWDDDNVEDDFSNQLRA |
| | TVLLMIKVYETPYGCYILHQKGRMCSAFLCC |
| 74 | MSEKKQPVDLGLLEEDDEFEEFPAEDWAGLDEDEDAHVWEDNWDDDNVEDDFSNQLRA |
| | TVLLMIKVYETPYGCYILHQKGRMCSAFLCC |
| 75 | SEKKQPVDLGLLEEDDEFEEFPAEDWAGLDEDEDAHVWEDNWDDDNVEDDFSNQLRAT |
| | VLLMILVAETPYGAYV |
| 76 | SEFEDANKEQGEVPNDDVSDELPKLLDDDEDELRGALFFQWHDWEEAEDNDWDVEDA |
| 77 | SELDFWDNWKDRQADLPAASDDEDFENDLAEVDHEDPLVDGNEWDGDEVEFELEEQK |
| 78 | SDADEFDDDNSVWGWEDDEVEPNDAFQPLGDVRANEELDEKDHKLDEEADLQWEFEL |
| 79 | SQWAPLDHLESDEWGAFEDEDFNNGDVEELDPEERDDEDLFQKDVLVAKADDEEWDN |
| 80 | STKKQPVDLGLLETDSEFTEFPATSWAGLSESESAHVWTSNWDSSNVTSDFSNQLRA |
| 81 | STKKQPVDLGLLETDDEFEEFPATSWAGLSEDESAHVWESNWDDSNVTSDFSNQLRA |
| 82 | STKKQPVDLGLLEEDDEFEEFPAESWAGLDEDESAHVWEDNWDDDNVTSDFSNQLRA |
| 83 | SEKKQPVDLGLLEEDDEFEEFPAEDWAGLDEDESAHVWEDNWDDDNVTSDFSNQLRA |
| 84 | GLAALALMVRQPAFALEIYMGVKHTALYVST |
| 85 | SLLAVFPTIEAARYKMGLLYHAVAATQGMLV |
| 86 | SEFEDANKEQGEVPNDDVSDELPKLLDDDEDELRGALFFQWHDWEEAEDNDWDVEDAT |
| | VLLMILVAETPYGAYVLHQKGRMASAFLAA |
| 87 | SELDFWDNWKDRQADLPAASDDEDFENDLAEVDHEDPLVDGNEWDGDEVEFELEEQKT |
| | VLLMILVAETPYGAYVLHQKGRMASAFLAA |
| 88 | SDADEFDDDNSVWGWEDDEVEPNDAFQPLGDVRANEELDEKDHKLDEEADLQWEFELT |
| | VLLMILVAETPYGAYVLHQKGRMASAFLAA |
| 89 | SQWAPLDHLESDEWGAFEDEDENNGDVEELDPEERDDEDLFQKDVLVAKADDEEWDNT |
| | VLLMILVAETPYGAYVLHQKGRMASAFLAA |
| 90 | STKKQPVDLGLLETDSEFTEFPATSWAGLSESESAHVWTSNWDSSNVTSDFSNQLRATVL |
| | LMILVAETPYGAYVLHQKGRMASAFLAA |
| 91 | STKKQPVDLGLLETDDEFEEFPATSWAGLSEDESAHVWESNWDDSNVTSDFSNQLRATVL |
| | LMILVAETPYGAYVLHQKGRMASAFLAA |
| 92 | STKKQPVDLGLLEEDDEFEEFPAESWAGLDEDESAHVWEDNWDDDNVTSDFSNQLRATV |
| | LLMILVAETPYGAYVLHQKGRMASAFLAA |
| 93 | SEKKQPVDLGLLEEDDEFEEFPAEDWAGLDEDESAHVWEDNWDDDNVTSDFSNQLRAT |
| | VLLMILVAETPYGAYVLHQKGRMASAFLAA |
| 94 | SEKKQPVDLGLLEEDDEFEEFPAEDWAGLDEDEDAHVWEDNWDDDNVEDDFSNQLRAG |
| | LAALALMVRQPAFALEIYMGVKHTALYVST |
| 95 | SEKKQPVDLGLLEEDDEFEEFPAEDWAGLDEDEDAHVWEDNWDDDNVEDDFSNQLRAS |
| | LLAVFPTIEAARYKMGLLYHAVAATQGMLV |
| 96 | MSEKKQPVDLGLLEEDDEFEEFPAEDWAGLDEDEDAHVWEDNWDDDNVTSDFSNQLRA |
| 97 | TVLLMILVCETPYGCYVLHQKGRMC |
| 98 | TVLLMILVCETPYGCYVLHQKGRM |
| 99 | TVLLMILVCETPYGCYVLHQKGR |
| 100 | TVLLMILVCETPYGCYVLHQKG |
| 101 | TVLLMILVCETPYGCYVLH |
| 102 | TVLLMILVCETPYGCYVL |
| 103 | TVLLMILVCETPYGCY |
| 104 | MSEKKQPVDLGLLEEDDEFEEFPAEDWAGLDEDEDAHVWEDNWDDDNVTSDFSNQLRA |
| | TVLLMILVCETPYGCYVLHQKGRMC |
| 105 | MSEKKQPVDLGLLEEDDEFEEFPAEDWAGLDEDEDAHVWEDNWDDDNVTSDFSNQLRA |
| | TVLLMILVCETPYGCYVLHQKGRM |
| 106 | MSEKKQPVDLGLLEEDDEFEEFPAEDWAGLDEDEDAHVWEDNWDDDNVTSDFSNQLRA |
| | TVLLMILVCETPYGCYVLHQKGR |
| 107 | MSEKKQPVDLGLLEEDDEFEEFPAEDWAGLDEDEDAHVWEDNWDDDNVTSDFSNQLRA |
| | TVLLMILVCETPYGCYVLHQKG |
| 108 | MSEKKQPVDLGLLEEDDEFEEFPAEDWAGLDEDEDAHVWEDNWDDDNVTSDFSNQLRA |
| | TVLLMILVCETPYGCYVLH |
| 109 | MSEKKQPVDLGLLEEDDEFEEFPAEDWAGLDEDEDAHVWEDNWDDDNVTSDFSNQLRA |
| | TVLLMILVCETPYGCYVL |
| 110 | MSEKKQPVDLGLLEEDDEFEEFPAEDWAGLDEDEDAHVWEDNWDDDNVTSDFSNQLRA |
| | TVLLMILVCETPYGCY |

1. A method for treating or preventing allergic conjunctivitis in a subject in need thereof, the method comprising:
administering to the subject an effective amount of an artificial polypeptide of formula (I): 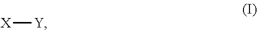
wherein:
X is a moiety comprising an amino acid sequence having at least 80% sequence identity with SEQ ID NO: 1, and
Y is a moiety comprising 10 to 50 amino acids, at least 50% of which are selected from the group consisting of: isoleucine (I), valine (V), leucine (L), phenylalanine (F), cysteine (C) methionine (M) and alanine (A); and
Y comprises a total number of C of less than 5.

2. The method of claim 1, wherein X comprises an amino acid sequence having at least 90% sequence identity with SEQ ID NO: 1.

3. The method of claim 1, wherein at least 50% amino acids of X are selected from the group consisting of: arginine (R), lysine (K), asparagine (N), aspartic acid (D), glutamine (Q), glutamic acid (E), and histidine (H).

4. The method of claim 1, wherein Y comprises a total number of C of less than 4.

5. The method of claim 1, wherein a total number of hydrophobic amino acids in Y is more than 8 and/or a total number of hydrophilic amino acids in Y is no more than 5.

6. The method of claim 1, wherein X comprises an amino acid sequence having at least 90% sequence identity with any one of SEQ ID NOs: 81-83.

7. The method of claim 1, wherein Y comprises an amino acid sequence having at least 90% sequence identity with any one of SEQ ID NOs: 3-18, 58-61, 84, or 85.

8. The method of claim 1, wherein the artificial polypeptide comprises an amino acid sequence having at least 90% sequence identity with any one of SEQ ID NOs: 29-41, 54-57, 75, or 91-95.

9. The method of claim 1, wherein the allergic conjunctivitis is at least one selected from the group consisting of: seasonal allergic conjunctivitis, perennial allergic conjunctivitis, vernal keratoconjunctivitis, giant papillary conjunctivitis, atopic keratoconjunctivitis, and phlyctenular keratoconjunctivitis.

10. The method of claim 9, wherein:
the seasonal allergic conjunctivitis is caused or partially caused by a seasonal allergen,
the perennial allergic conjunctivitis is caused or partially caused by a perennial allergen;
the vernal keratoconjunctivitis is at least one selected from the group consisting of palpebral vernal keratoconjunctivitis, limbal vernal keratoconjunctivitis, and mixed vernal keratoconjunctivitis;
the giant papillary conjunctivitis is caused or partially caused by repeated mechanical irritation of the conjunctiva;
the atopic keratoconjunctivitis is accompanied with atopic dermatitis and/or asthma; or
the phlyctenular keratoconjunctivitis is caused or partially caused by a delayed hypersensitivity or inflammatory reaction of the cornea and conjunctiva to antigens expressed by various pathogens.

11. The method of claim 10, wherein the seasonal allergen is at least one selected from the group consisting of: tree pollens, weed pollens, grass pollens, and mold spores.

12. (canceled)

13. The method of claim 10, wherein the perennial allergen is at least one selected from the group consisting of: dust mites, cockroaches, and animal danders.

14. (canceled)

15. (canceled)

16. The method of claim 10, wherein the repeated mechanical irritation of the conjunctiva is associated with at least one selected from the group consisting of: contact lenses wearing, use of ocular prostheses, use of conjunctival sutures, use of extruding scleral buckles, and elevated corneal scars.

17. (canceled)

18. (canceled)

19. The method of claim 10, wherein the pathogens are at least one selected from the group consisting of: bacteria, viruses, chlamydiae, fungi, and parasites.

20. The method of claim 1, wherein the subject does not have any ocular disease selected from the group consisting of viral conjunctivitis, bacterial conjunctivitis, chemical conjunctivitis, and dry eye or dry eye associated disorders.

21. (canceled)

22. The method of claim 1, wherein:
the subject displays reduction in at least one symptom selected from the group consisting of redness of conjunctiva, edema of conjunctiva, hyperemia of conjunctiva, redness of eyelids, edema of eyelids, hyperemia of eyelids, ocular itching, visible damage of cornea, mucous discharge, watery discharge, tearing, photophobia, foreign body sensation, and difficulty in opening eyes, after administrating of at least one dose of the artificial polypeptide of formula (I); or
the subject displays improvements in at least one tissue selected from the group consisting of: conjunctiva, cornea, anterior chamber, iris, pupil, vitreum vitreous, and fundus, after administrating of at least one dose of the artificial polypeptide of formula (I).

23. (canceled)

24. A method for treating or preventing allergic conjunctivitis in a subject in need thereof, comprising administering to the subject an effective amount of an artificial polypeptide of formula (II): 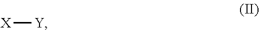
wherein X is a moiety comprising a sequence having at least 80% identity with SEQ ID NO: 1, and
Y is a moiety comprising 10 to 50 amino acids, at least 50% of which are selected from I, V, L, F, C, M, and A;
wherein Y comprises a sequence having at most 90% identity with SEQ ID NO: 2.

25.-45. (canceled)

46. A method for treating or preventing allergic conjunctivitis in a subject in need thereof, comprising administering to the subject an effective amount of an artificial polypeptide of formula (III):
X—Y(III),
wherein X is a moiety comprising 40 to 65 amino acids, at least 50% of which are selected from H, R, K, D, Q, N and E; and
Y is a moiety comprising a sequence having at least 80% identity with SEQ ID NO: 2,
wherein a total number of H, R, K, D, Q, N and E in X is less than 33.

47.-285. (canceled)


SEQUENCE LISTING (110 sequences)

SEQ ID NO: 1 - protein, 57 aa, synthetic construct
SEKKQPVDLGLLEEDDEFEEFPAEDWAGLDEDEDAHVWEDNWDDDNVEDDFSNQLRA

SEQ ID NO: 2 - protein, 31 aa, synthetic construct
TVLLMILVCETPYGCYVLHQKGRMCSAFLCC

SEQ ID NO: 3 - protein, 31 aa, synthetic construct
TVLLMILVAETPYGAYVLHQKGRMASAFLAA

SEQ ID NO: 4 - protein, 31 aa, synthetic construct
TVLLMILVCETPYGCYVLHQKGRMCSAFLCA

SEQ ID NO: 5 - protein, 31 aa, synthetic construct
TVLLMILVVETPYGVYVLHQKGRMVSAFLVV

SEQ ID NO: 6 - protein, 31 aa, synthetic construct
TVLLMILVAETPYGAYVLHQKGRMVSAFLVV

SEQ ID NO: 7 - protein, 31 aa, synthetic construct
TVLLMILVAETPYGVYVLHQKGRMASAFLVV

SEQ ID NO: 8 - protein, 31 aa, synthetic construct
TVLLMILVAETPYGVYVLHQKGRMVSAFLAV

SEQ ID NO: 9 - protein, 30 aa, synthetic construct
VLLMILVAETPYGVYVLHQKGRMVSAFLVA

SEQ ID NO: 10 - protein, 31 aa, synthetic construct
TVLLMILVVETPYGAYVLHQKGRMASAFLVV

SEQ ID NO: 11 - protein, 31 aa, synthetic construct
TVLLMILVVETPYGAYVLHQKGRMVSAFLAV

SEQ ID NO: 12 - protein, 31 aa, synthetic construct
TVLLMILVVETPYGAYVLHQKGRMVSAFLVA

SEQ ID NO: 13 - protein, 31 aa, synthetic construct
TVLLMILVVETPYGVYVLHQKGRMASAFLAV

SEQ ID NO: 14 - protein, 31 aa, synthetic construct; identical to SEQ ID NO: 13

SEQ ID NO: 15 - protein, 31 aa, synthetic construct
TVLLMILVVETPYGVYVLHQKGRMVSAFLAA

SEQ ID NO: 16 - protein, 31 aa, synthetic construct
TVLLVILVAETPYGAYVLHQKGRVASAFLAA

SEQ ID NO: 17 - protein, 31 aa, synthetic construct
GLCALCLMVRQPCFCLEIYMGVKHTCLYVST

SEQ ID NO: 18 - protein, 31 aa, synthetic construct
SLLCVFPTIECCRYKMGLLYHCVACTQGMLV

SEQ ID NO: 19 - protein, 57 aa, synthetic construct
SEFEDANKEQGEVPNDDVSDELPKLLDDDEDELRGALFFQWHDWEEAEDNDWDVEDA

SEQ ID NO: 20 - protein, 57 aa, synthetic construct
SELDFWDNWKDRQADLPAASDDEDFENDLAEVDHEDPLVDGNEWDGDEVEFELEEQK

SEQ ID NO: 21 - protein, 57 aa, synthetic construct
SDADEFDDDNSVWGWEDDEVEPNDAFQPLGDVRANEELDEKDHKLDEEADLQWEFEL

SEQ ID NO: 22 - protein, 57 aa, synthetic construct
SQWAPLDHLESDEWGAFEDEDFNNGDVEELDPEERDDEDLFQKDVLVAKADDEEWDN

SEQ ID NO: 23 - protein, 57 aa, synthetic construct
STKKQPVDLGLLETDSEFTEFPATSWAGLSESESAHVWTSNWDSSNVTSDFSNQLRA

SEQ ID NO: 24 - protein, 57 aa, synthetic construct
STKKQPVDLGLLETDDEFEEFPATSWAGLSEDESAHVWESNWDDSNVTSDFSNQLRA

SEQ ID NO: 25 - protein, 57 aa, synthetic construct
STKKQPVDLGLLEEDDEFEEFPAESWAGLDEDESAHVWEDNWDDDNVTSDFSNQLRA

SEQ ID NO: 26 - protein, 57 aa, synthetic construct
SEKKQPVDLGLLEEDDEFEEFPAEDWAGLDEDESAHVWEDNWDDDNVTSDFSNQLRA

SEQ ID NO: 27 - protein, 57 aa, synthetic construct
SEKKQPVDLGLLEEDDEFEEFPAEDWAGLDEDEDAHVWEDNWDDDNVTSDFSNQLRA

SEQ ID NO: 28 - protein, 88 aa, synthetic construct
SEKKQPVDLGLLEEDDEFEEFPAEDWAGLDEDEDAHVWEDNWDDDNVEDDFSNQLRATVLLMILVCETPYGCYVLHQKGRMCSAFLCC

SEQ ID NO: 29 - protein, 88 aa, synthetic construct
SEKKQPVDLGLLEEDDEFEEFPAEDWAGLDEDEDAHVWEDNWDDDNVEDDFSNQLRATVLLMILVAETPYGAYVLHQKGRMASAFLAA

SEQ ID NO: 30 - protein, 88 aa, synthetic construct
SEKKQPVDLGLLEEDDEFEEFPAEDWAGLDEDEDAHVWEDNWDDDNVEDDFSNQLRATVLLMILVCETPYGCYVLHQKGRMCSAFLCA

SEQ ID NO: 31 - protein, 88 aa, synthetic construct
SEKKQPVDLGLLEEDDEFEEFPAEDWAGLDEDEDAHVWEDNWDDDNVEDDFSNQLRATVLLMILVVETPYGVYVLHQKGRMVSAFLVV

SEQ ID NO: 32 - protein, 88 aa, synthetic construct
SEKKQPVDLGLLEEDDEFEEFPAEDWAGLDEDEDAHVWEDNWDDDNVEDDFSNQLRATVLLMILVAETPYGAYVLHQKGRMVSAFLVV

SEQ ID NO: 33 - protein, 88 aa, synthetic construct
SEKKQPVDLGLLEEDDEFEEFPAEDWAGLDEDEDAHVWEDNWDDDNVEDDFSNQLRATVLLMILVAETPYGVYVLHQKGRMASAFLVV

SEQ ID NO: 34 - protein, 88 aa, synthetic construct
SEKKQPVDLGLLEEDDEFEEFPAEDWAGLDEDEDAHVWEDNWDDDNVEDDFSNQLRATVLLMILVAETPYGVYVLHQKGRMVSAFLAV

SEQ ID NO: 35 - protein, 88 aa, synthetic construct
SEKKQPVDLGLLEEDDEFEEFPAEDWAGLDEDEDAHVWEDNWDDDNVEDDFSNQLRATVLLMILVAETPYGVYVLHQKGRMVSAFLVA

SEQ ID NO: 36 - protein, 88 aa, synthetic construct
SEKKQPVDLGLLEEDDEFEEFPAEDWAGLDEDEDAHVWEDNWDDDNVEDDFSNQLRATVLLMILVVETPYGAYVLHQKGRMASAFLVV

SEQ ID NO: 37 - protein, 88 aa, synthetic construct
SEKKQPVDLGLLEEDDEFEEFPAEDWAGLDEDEDAHVWEDNWDDDNVEDDFSNQLRATVLLMILVVETPYGAYVLHQKGRMVSAFLAV

SEQ ID NO: 38 - protein, 88 aa, synthetic construct
SEKKQPVDLGLLEEDDEFEEFPAEDWAGLDEDEDAHVWEDNWDDDNVEDDFSNQLRATVLLMILVVETPYGAYVLHQKGRMVSAFLVA

SEQ ID NO: 39 - protein, 88 aa, synthetic construct
SEKKQPVDLGLLEEDDEFEEFPAEDWAGLDEDEDAHVWEDNWDDDNVEDDFSNQLRATVLLMILVVETPYGVYVLHQKGRMASAFLAV

SEQ ID NO: 40 - protein, 88 aa, synthetic construct
SEKKQPVDLGLLEEDDEFEEFPAEDWAGLDEDEDAHVWEDNWDDDNVEDDFSNQLRATVLLMILVVETPYGVYVLHQKGRMASAFLVA

SEQ ID NO: 41 - protein, 88 aa, synthetic construct
SEKKQPVDLGLLEEDDEFEEFPAEDWAGLDEDEDAHVWEDNWDDDNVEDDFSNQLRATVLLMILVVETPYGVYVLHQKGRMVSAFLAA

SEQ ID NO: 42 - protein, 88 aa, synthetic construct
SEKKQPVDLGLLEEDDEFEEFPAEDWAGLDEDEDAHVWEDNWDDDNVEDDFSNQLRAGLCALCLMVRQPCFCLEIYMGVKHTCLYVST

SEQ ID NO: 43 - protein, 88 aa, synthetic construct
SEKKQPVDLGLLEEDDEFEEFPAEDWAGLDEDEDAHVWEDNWDDDNVEDDFSNQLRASLLCVFPTIECCRYKMGLLYHCVACTQGMLV

SEQ ID NO: 44 - protein, 88 aa, synthetic construct
SEKKQPVDLGLLEEDDEFEEFPAEDWAGLDEDEDAHVWEDNWDDDNVEDDFSNQLRATVLLVILVAETPYGAYVLHQKGRVASAFLAA

SEQ ID NO: 45 - protein, 88 aa, synthetic construct
SEFEDANKEQGEVPNDDVSDELPKLLDDDEDELRGALFFQWHDWEEAEDNDWDVEDATVLLMILVCETPYGCYVLHQKGRMCSAFLCC

SEQ ID NO: 46 - protein, 88 aa, synthetic construct
SELDFWDNWKDRQADLPAASDDEDFENDLAEVDHEDPLVDGNEWDGDEVEFELEEQKTVLLMILVCETPYGCYVLHQKGRMCSAFLCC

SEQ ID NO: 47 - protein, 88 aa, synthetic construct
SDADEFDDDNSVWGWEDDEVEPNDAFQPLGDVRANEELDEKDHKLDEEADLQWEFELTVLLMILVCETPYGCYVLHQKGRMCSAFLCC

SEQ ID NO: 48 - protein, 88 aa, synthetic construct
SQWAPLDHLESDEWGAFEDEDFNNGDVEELDPEERDDEDLFQKDVLVAKADDEEWDNTVLLMILVCETPYGCYVLHQKGRMCSAFLCC

SEQ ID NO: 49 - protein, 88 aa, synthetic construct
STKKQPVDLGLLETDSEFTEFPATSWAGLSESESAHVWTSNWDSSNVTSDFSNQLRATVLLMILVCETPYGCYVLHQKGRMCSAFLCC

SEQ ID NO: 50 - protein, 88 aa, synthetic construct
STKKQPVDLGLLETDDEFEEFPATSWAGLSEDESAHVWESNWDDSNVTSDFSNQLRATVLLMILVCETPYGCYVLHQKGRMCSAFLCC

SEQ ID NO: 51 - protein, 88 aa, synthetic construct
STKKQPVDLGLLEEDDEFEEFPAESWAGLDEDESAHVWEDNWDDDNVTSDFSNQLRATVLLMILVCETPYGCYVLHQKGRMCSAFLCC

SEQ ID NO: 52 - protein, 88 aa, synthetic construct
SEKKQPVDLGLLEEDDEFEEFPAEDWAGLDEDESAHVWEDNWDDDNVTSDFSNQLRATVLLMILVCETPYGCYVLHQKGRMCSAFLCC

SEQ ID NO: 53 - protein, 88 aa, synthetic construct
SEKKQPVDLGLLEEDDEFEEFPAEDWAGLDEDEDAHVWEDNWDDDNVTSDFSNQLRATVLLMILVCETPYGCYVLHQKGRMCSAFLCC

SEQ ID NO: 54 - protein, 83 aa, synthetic construct
SEKKQPVDLGLLEEDDEFEEFPAEDWAGLDEDEDAHVWEDNWDDDNVTSDFSNQLRATVLLMILVCETPYGCYVLHQKGRMCS

SEQ ID NO: 55 - protein, 78 aa, synthetic construct
SEKKQPVDLGLLEEDDEFEEFPAEDWAGLDEDEDAHVWEDNWDDDNVTSDFSNQLRATVLLMILVCETPYGCYVLHQK

SEQ ID NO: 56 - protein, 68 aa, synthetic construct
SEKKQPVDLGLLEEDDEFEEFPAEDWAGLDEDEDAHVWEDNWDDDNVTSDFSNQLRATVLLMILVCET

SEQ ID NO: 57 - protein, 63 aa, synthetic construct
SEKKQPVDLGLLEEDDEFEEFPAEDWAGLDEDEDAHVWEDNWDDDNVTSDFSNQLRATVLLMI

SEQ ID NO: 58 - protein, 57 aa, synthetic construct; identical to SEQ ID NO: 27

SEQ ID NO: 59 - protein, 57 aa, synthetic construct; identical to SEQ ID NO: 27

SEQ ID NO: 60 - protein, 57 aa, synthetic construct; identical to SEQ ID NO: 27

SEQ ID NO: 61 - protein, 57 aa, synthetic construct; identical to SEQ ID NO: 27

SEQ ID NO: 62 - protein, 69 aa, synthetic construct
SEKKQPVDLGLLEEDDEFEEFPAEDWAGLDEDEDAHVWEDNWDDDNVEDDFSNQLRAELEKHGYKMETS

SEQ ID NO: 63 - protein, 89 aa, synthetic construct
MSEKKQPVDLGLLEEDDEFEEFPAEDWAGLDEDEDAHVWEDNWDDDNVEDDFSNQLRATVLLMILVCETPYGCYVLHQKGRMCSAFLCC

SEQ ID NO: 64 - protein, 89 aa, synthetic construct; identical to SEQ ID NO: 63

SEQ ID NO: 65 - protein, 89 aa, synthetic construct
MSEKKQPVDLGLLEEDDEFEEFPAEDWAGLDEDEDAHVWEDNWDDDNVEDDFSNQLRVTVLLMILVCETLYGCYVLHQKGRMCSAFLCC

SEQ ID NO: 66 - protein, 89 aa, synthetic construct
MSEKKQPVDLGLLEEDDEFEEFPAEDWAGLDEDEDAHVWEDNWDDDNVEDDFSNQLRATILLMILVCETPYGCYVLHQKGRMCSAFLCC

SEQ ID NO: 67 - protein, 89 aa, synthetic construct
MSEKKQPVDLGLLEEDDEFEEFPAEDWAGLDEDEDAHVWEDNWDDDNVEDDFSNQLRATVLLMVLVCETPYGCYVLHQKERMCSAFLCC

SEQ ID NO: 68 - protein, 89 aa, synthetic construct
MSEKKQPVDLGLLEEDDEFEEFPAEDWAGLDEDEDAHVWEDNWDDDNVEDDFSNQLRATVLLMIKVYETPYGCYILHQKGRMCSAFLCC

SEQ ID NO: 69 - protein, 89 aa, synthetic construct; identical to SEQ ID NO: 68

SEQ ID NO: 70 - protein, 89 aa, synthetic construct; identical to SEQ ID NO: 68

SEQ ID NO: 71 - protein, 89 aa, synthetic construct
MSEKKQPVDLGLLEEDDEFEEFPAEDWAGLDEDEDAHVWEDNWDDDNVEDDFSNQLRATVLLMKKVYETPYGCYILHQKGRMCSAFLCC

SEQ ID NO: 72 - protein, 89 aa, synthetic construct; identical to SEQ ID NO: 68

SEQ ID NO: 73 - protein, 89 aa, synthetic construct; identical to SEQ ID NO: 68

SEQ ID NO: 74 - protein, 89 aa, synthetic construct; identical to SEQ ID NO: 68

SEQ ID NO: 75 - protein, 74 aa, synthetic construct
SEKKQPVDLGLLEEDDEFEEFPAEDWAGLDEDEDAHVWEDNWDDDNVEDDFSNQLRATVLLMILVAETPYGAYV

SEQ ID NO: 76 - protein, 57 aa, synthetic construct; identical to SEQ ID NO: 19

SEQ ID NO: 77 - protein, 57 aa, synthetic construct; identical to SEQ ID NO: 20

SEQ ID NO: 78 - protein, 57 aa, synthetic construct; identical to SEQ ID NO: 21

SEQ ID NO: 79 - protein, 57 aa, synthetic construct; identical to SEQ ID NO: 22

SEQ ID NO: 80 - protein, 57 aa, synthetic construct; identical to SEQ ID NO: 23

SEQ ID NO: 81 - protein, 57 aa, synthetic construct; identical to SEQ ID NO: 24

SEQ ID NO: 82 - protein, 57 aa, synthetic construct; identical to SEQ ID NO: 25

SEQ ID NO: 83 - protein, 57 aa, synthetic construct; identical to SEQ ID NO: 26

SEQ ID NO: 84 - protein, 31 aa, synthetic construct
GLAALALMVRQPAFALEIYMGVKHTALYVST

SEQ ID NO: 85 - protein, 31 aa, synthetic construct
SLLAVFPTIEAARYKMGLLYHAVAATQGMLV

SEQ ID NO: 86 - protein, 88 aa, synthetic construct
SEFEDANKEQGEVPNDDVSDELPKLLDDDEDELRGALFFQWHDWEEAEDNDWDVEDATVLLMILVAETPYGAYVLHQKGRMASAFLAA

SEQ ID NO: 87 - protein, 88 aa, synthetic construct
SELDFWDNWKDRQADLPAASDDEDFENDLAEVDHEDPLVDGNEWDGDEVEFELEEQKTVLLMILVAETPYGAYVLHQKGRMASAFLAA

SEQ ID NO: 88 - protein, 88 aa, synthetic construct
SDADEFDDDNSVWGWEDDEVEPNDAFQPLGDVRANEELDEKDHKLDEEADLQWEFELTVLLMILVAETPYGAYVLHQKGRMASAFLAA

SEQ ID NO: 89 - protein, 88 aa, synthetic construct
SQWAPLDHLESDEWGAFEDEDFNNGDVEELDPEERDDEDLFQKDVLVAKADDEEWDNTVLLMILVAETPYGAYVLHQKGRMASAFLAA

SEQ ID NO: 90 - protein, 88 aa, synthetic construct
STKKQPVDLGLLETDSEFTEFPATSWAGLSESESAHVWTSNWDSSNVTSDFSNQLRATVLLMILVAETPYGAYVLHQKGRMASAFLAA

SEQ ID NO: 91 - protein, 88 aa, synthetic construct
STKKQPVDLGLLETDDEFEEFPATSWAGLSEDESAHVWESNWDDSNVTSDFSNQLRATVLLMILVAETPYGAYVLHQKGRMASAFLAA

SEQ ID NO: 92 - protein, 88 aa, synthetic construct
STKKQPVDLGLLEEDDEFEEFPAESWAGLDEDESAHVWEDNWDDDNVTSDFSNQLRATVLLMILVAETPYGAYVLHQKGRMASAFLAA

SEQ ID NO: 93 - protein, 88 aa, synthetic construct
SEKKQPVDLGLLEEDDEFEEFPAEDWAGLDEDESAHVWEDNWDDDNVTSDFSNQLRATVLLMILVAETPYGAYVLHQKGRMASAFLAA

SEQ ID NO: 94 - protein, 88 aa, synthetic construct
SEKKQPVDLGLLEEDDEFEEFPAEDWAGLDEDEDAHVWEDNWDDDNVEDDFSNQLRAGLAALALMVRQPAFALEIYMGVKHTALYVST

SEQ ID NO: 95 - protein, 88 aa, synthetic construct
SEKKQPVDLGLLEEDDEFEEFPAEDWAGLDEDEDAHVWEDNWDDDNVEDDFSNQLRASLLAVFPTIEAARYKMGLLYHAVAATQGMLV

SEQ ID NO: 96 - protein, 58 aa, synthetic construct
MSEKKQPVDLGLLEEDDEFEEFPAEDWAGLDEDEDAHVWEDNWDDDNVTSDFSNQLRA

SEQ ID NO: 97 - protein, 25 aa, synthetic construct
TVLLMILVCETPYGCYVLHQKGRMC

SEQ ID NO: 98 - protein, 24 aa, synthetic construct
TVLLMILVCETPYGCYVLHQKGRM

SEQ ID NO: 99 - protein, 23 aa, synthetic construct
TVLLMILVCETPYGCYVLHQKGR

SEQ ID NO: 100 - protein, 22 aa, synthetic construct
TVLLMILVCETPYGCYVLHQKG

SEQ ID NO: 101 - protein, 19 aa, synthetic construct
TVLLMILVCETPYGCYVLH

SEQ ID NO: 102 - protein, 18 aa, synthetic construct
TVLLMILVCETPYGCYVL

SEQ ID NO: 103 - protein, 16 aa, synthetic construct
TVLLMILVCETPYGCY

SEQ ID NO: 104 - protein, 83 aa, synthetic construct
MSEKKQPVDLGLLEEDDEFEEFPAEDWAGLDEDEDAHVWEDNWDDDNVTSDFSNQLRATVLLMILVCETPYGCYVLHQKGRMC

SEQ ID NO: 105 - protein, 82 aa, synthetic construct
MSEKKQPVDLGLLEEDDEFEEFPAEDWAGLDEDEDAHVWEDNWDDDNVTSDFSNQLRATVLLMILVCETPYGCYVLHQKGRM

SEQ ID NO: 106 - protein, 81 aa, synthetic construct
MSEKKQPVDLGLLEEDDEFEEFPAEDWAGLDEDEDAHVWEDNWDDDNVTSDFSNQLRATVLLMILVCETPYGCYVLHQKGR

SEQ ID NO: 107 - protein, 80 aa, synthetic construct
MSEKKQPVDLGLLEEDDEFEEFPAEDWAGLDEDEDAHVWEDNWDDDNVTSDFSNQLRATVLLMILVCETPYGCYVLHQKG

SEQ ID NO: 108 - protein, 77 aa, synthetic construct
MSEKKQPVDLGLLEEDDEFEEFPAEDWAGLDEDEDAHVWEDNWDDDNVTSDFSNQLRATVLLMILVCETPYGCYVLH

SEQ ID NO: 109 - protein, 76 aa, synthetic construct
MSEKKQPVDLGLLEEDDEFEEFPAEDWAGLDEDEDAHVWEDNWDDDNVTSDFSNQLRATVLLMILVCETPYGCYVL

SEQ ID NO: 110 - protein, 74 aa, synthetic construct
MSEKKQPVDLGLLEEDDEFEEFPAEDWAGLDEDEDAHVWEDNWDDDNVTSDFSNQLRATVLLMILVCETPYGCY